SEQUENCE LISTING (25 sequences)

SEQ ID NO: 1 - protein, 308 aa, Homo sapiens
MWKRWLALALALVAVAWVRAEEELRSKSKICANVFCGAGRECAVTEKGEPTCLCIEQCKPHKRPVCGSNGKTYLNHCELHRDACLTGSKIQVDYDGHCKEKKSVSPSASPVVCYQSNRDELRRRIIQWLEAEIIPDGWFSKGSNYSEILDKYFKNFDNGDSRLDSSEFLKFVEQNETAINITTYPDQENNKLLRGLCVDALIELSDENADWKLSFQEFLKCLNPSFNPPEKKCALEDETYADGAETEVDCNRCVCACGNWVCTAMTCDGKNQKGAQTQTEEEMTRYVQELQKHQETAEKTKRVSTKEI

SEQ ID NO: 2 - DNA, 3705 bp, Homo sapiens
gatcggcggagctcccacctccgcttacagctcgctgccgccgtcctgccccgcgcccccaggagacctggaccagaccacgatgtggaaacgctggctcgcgctcgcgctcgcgctggtggcggtcgcctgggtccgcgccgaggaagagctaaggagcaaatccaagatctgtgccaatgtgttttgtggagccggccgggaatgtgcagtcacagagaaaggggaacccacctgtctctgcattgagcaatgcaaacctcacaagaggcctgtgtgtggcagtaatggcaagacctacctcaaccactgtgaactgcatcgagatgcctgcctcactggatccaaaatccaggttgattacgatggacactgcaaagagaagaaatccgtaagtccatctgccagcccagttgtttgctatcagtccaaccgtgatgagctccgacgtcgcatcatccagtggctggaagctgagatcattccagatggctggttctctaaaggcagcaactacagtgaaatcctagacaagtattttaagaactttgataatggtgattctcgcctggactccagtgaattcctgaagtttgtggaacagaatgaaactgccatcaatattacaacgtatccagaccaggagaacaacaagttgcttaggggactctgtgttgatgctctcattgaactgtctgatgaaaatgctgattggaaactcagcttccaagagtttctcaagtgcctcaacccatctttcaaccctcctgagaagaagtgtgccctggaggatgaaacgtatgcagatggagctgagaccgaggtggactgtaaccgctgtgtctgtgcctgtggaaattgggtctgtacagccatgacctgtgacggaaagaatcagaagggggcccagacccagacagaggaggagatgaccagatatgtccaggagctccaaaagcatcaggaaacagctgaaaagaccaagagagtgagcaccaaagagatctaatgaggaggcacagaccagtgtctggatcccagcatcttctccacttcagcgctgagttcagtatacacaagtgtctgctacagtcgccaaatcaccagtatttgcttatatagcaatgagttttattttgtttatttgttttgcaataaaggatatgaaggtggctggctaggaagggaagggccacagccttcatttctaggagtgctttaagagaaactgtaaatggtgctctggggctggaggctagtaaggaaactgcatcacgattgaaagaggaacagacccaaatctgaacctcttttgagtttactgcatctgtcagcaggctgcagggagtgcacacgatgccagagagaacttagcagggtgtccccggaggagaggtttgggaagctccacggagaggaacgctctctgcttccagcctctttccattgccgtcagcatgacagacctccagcatccacgcatctcttggtcccaataactgcctctagatacatagccatactgctagttaacccagtgtccctcagacttggatggagtttctgggagggtacacccaaatgatgcagatacttgtatactttgagccccttagcgacctaaccaaattttaaaaatactttttaccaaaggtgctatttctctgtaaaacactttttttttggcaagttgactttattcttcaattattatcattatattattgttttttaatattttattttcttgactaggtattaagcttttgtaattatttttcagtagtcccaccacttcataggtggaaggagtttggggttcttcctggtgcaggggctgaaataacccagatgcccccaccctgccacatactagatgcagcccatagttggcccccctagcttccagcagtccactatctgccagaggagcaagggtgccttagaccgaagccaggggaagaagcatcttcataaaaaactttcaagatccaaacattaatttgtttttatttattctgagaagttgaggcaaatcagtattcccaaggatggcgacaagggcagccaagcagggcttaggatatcccagcctaccaatatgctcattcgactaactaggagggtgagttggccctgtctcttcttttttctggacctcagtttcctcagtgagctggtaagaatgcactaaccttttgatttgataagttataaattctgtggttctgatcattggtccagaggggagataggttcctgtgatttttccttcttctctatagaataaatgaaatcttgttactagaacaagaaatgtcagatggccaaaaacaagatgaccagatttgatctcagcctgatgaccctacaggtcgtgctatgatatggagtcctcatgggtaaagcaggaagagagtgggaaagagaaccaccccactctgtcttcatatttgcatttcatgtttaacctccggctggaaatagaaagcattcccttagagatgaggataaaagaaagtttcagattcaacagggggaagaaaatggagatttaatcctaaaactgtgacttggggaggtcagtcatttacagttagtcctgtgtctttcgacttctgtgattattaaccccactcactaccctgtttcagatgcatttggaataccaaagattaaatccttgacataagatctcatttgcagaaagcagattaaagaccatcagaaggaaattatttaggttgtaatgcacaggcaactgtgagaaactgttgtgccaaaaatagaattccttctagtttttcttgttctcatttgaaaggagaaaattccactttgtttagcatttcaagcttttatgtatccatcccatctaaaaactcttcaaactccacttgttcagtctgaaatgcagctccctgtccaagtgccttggagaactcacagcagcacgccttaatcaaaggttttaccagcccttggacactatgggaggagggcaagagtacaccaatttgttaaaagcaagaaaccacagtgtctcttcactagtcatttagaacatggttatcatccaagactactctaccctgcaacattgaactcccaagagcaaatccacattcctcttgagttctgcagcttctgtgtaaatagggcagctgtcgtctatgccgtagaatcacatgatctgaggaccattcatggaagctgctaaatagcctagtctggggagtcttccataaagttttgcatggagcaaacaaacaggattaaactaggtttggttccttcagccctctaaaagcatagggcttagcctgcaggcttccttgggctttctctgtgtgtgtagttttgtaaacactatagcatctgttaagatccagtgtccatggaaacattcccacatgccgtgactctggactatatcagtttttggaaagcagggttcctctgcctgctaacaagcccacgtggaccagtctgaatgtctttcctttacacctatgtttttaagtagtcaaacttcaagaaacaatctaaacaagtttctgttgcatatgtgtttgtgaacttgtatttgtatttagtaggcttctatattgcatttaacttgtttttgtaactcctgattcttccttttcggatactattgatgaataaagaaattaaagtgaaaaaaaaaaaaaaaaaaaaaaa

SEQ ID NO: 3 - DNA, 19 bp, Artificial Sequence
  Description of Artificial Sequence Synthetic primer
tcaccaccatggagaaggc

SEQ ID NO: 4 - DNA, 20 bp, Artificial Sequence
  Description of Artificial Sequence Synthetic primer
gctaagcagttggtggtgca

SEQ ID NO: 5 - DNA, 25 bp, Artificial Sequence
  Description of Artificial Sequence Synthetic primer
aacagccatcaacatcaccacttat

SEQ ID NO: 6 - DNA, 22 bp, Artificial Sequence
  Description of Artificial Sequence Synthetic primer
tttccagtcagcgttctcatca

SEQ ID NO: 7 - DNA, 21 bp, Artificial Sequence
  Description of Artificial Sequence Synthetic primer
cgaggaggatgtgaacgacaa

SEQ ID NO: 8 - DNA, 19 bp, Artificial Sequence
  Description of Artificial Sequence Synthetic primer
ggtccgcagtccacgttct

SEQ ID NO: 9 - DNA, 18 bp, Artificial Sequence
  Description of Artificial Sequence Synthetic primer
caaccccggccaagaact

SEQ ID NO: 10 - DNA, 23 bp, Artificial Sequence
  Description of Artificial Sequence Synthetic primer
cttcctcctctgctggtactttg

SEQ ID NO: 11 - DNA, 20 bp, Artificial Sequence
  Description of Artificial Sequence Synthetic primer
attggcgagtttgagaaggt

SEQ ID NO: 12 - DNA, 20 bp, Artificial Sequence
  Description of Artificial Sequence Synthetic primer
tttgcatggtccgatgtagt

SEQ ID NO: 13 - DNA, 22 bp, Artificial Sequence
  Description of Artificial Sequence Synthetic primer
aatcaagaacgaaagtcggagg

SEQ ID NO: 14 - DNA, 19 bp, Artificial Sequence
  Description of Artificial Sequence Synthetic primer
gcgggtcatgggaataacg

SEQ ID NO: 15 - DNA, 21 bp, Artificial Sequence
  Description of Artificial Sequence Synthetic primer
ctgaaaattcgtggagcagaa

SEQ ID NO: 16 - DNA, 20 bp, Artificial Sequence
  Description of Artificial Sequence Synthetic primer
gtttccagtcagcgttatca

SEQ ID NO: 17 - RNA, 21 nt, Artificial Sequence
  Description of Artificial Sequence Synthetic oligonucleotide
ugcaaauacuuacggacuuuu

SEQ ID NO: 18 - RNA, 21 nt, Artificial Sequence
  Description of Artificial Sequence Synthetic oligonucleotide
ccgucaacaucaccgcuuauu

SEQ ID NO: 19 - RNA, 21 nt, Artificial Sequence
  Description of Artificial Sequence Synthetic oligonucleotide
uggcuaacgggcagagcgauu

SEQ ID NO: 20 - DNA, 21 bp, Artificial Sequence
  Description of Artificial Sequence Synthetic oligonucleotide
gcaucuugagauuuaaucatt

SEQ ID NO: 21 - DNA, 25 bp, Artificial Sequence
  Description of Artificial Sequence Synthetic primer
aacagccatcaacatcaccacttat

SEQ ID NO: 22 - DNA, 22 bp, Artificial Sequence
  Description of Artificial Sequence Synthetic primer
tttccagtcagcgttctcatca

SEQ ID NO: 23 - DNA, 19 bp, Artificial Sequence
  Description of Artificial Sequence Synthetic primer
tcaccaccatggagaaggc

SEQ ID NO: 24 - DNA, 20 bp, Artificial Sequence
  Description of Artificial Sequence Synthetic primer
gctaagcagttggtggtgca

SEQ ID NO: 25 - RNA, 21 nt, Artificial Sequence
  Description of Artificial Sequence Synthetic oligonucleotide
cagauggagcugagaccgauu

(19) United States
(12) Patent Application Publication
(10) Pub. No.: US 2009/0326053 A1
(43) Pub. Date: Dec. 31, 2009

(54) DIAGNOSTIC USES OF FOLLISTATIN-LIKE 1

(75) Inventors: Kenneth Walsh, Carlisle, MA (US); Noriyuki Ouchi, Boston, MA (US); Yuichi Oshima, Brookline, MA (US)

Correspondence Address: RONALD I. EISENSTEIN, 100 SUMMER STREET, NIXON PEABODY LLP, BOSTON, MA, 02110, US

(73) Assignee: TRUSTEES OF BOSTON UNIVERSITY, Boston, MA (US)

(21) Appl. No.: 12/475,175

(22) Filed: May 29, 2009

Related U.S. Application Data
(60) Provisional application No. 61/057,575, filed on May 30, 2008.

(51) Int. Cl. A61K 31/711; C12M 1/34; A61P 9/10

(52) U.S. Cl. 514/44 R; 435/287.1; 435/287.2

(57) ABSTRACT

The present invention generally relates to methods, systems and computer readable media for the diagnosis and/or prognosis of a cardiac stress and/or skeletal muscle stress in a subject. In particular, in one embodiment, the methods, systems and computer readable media detect a level of Fstl expression, such as Fstl1 polypeptide or mRNA expression in a biological sample obtained from a subject, where a high level relative to a reference Fstl expression level is indicative of a subject having, or is at risk of cardiac stress and/or skeletal muscle stress. In such embodiments, the method futher comprises administering or undertaking an appropriate therapy in a subject identified to have or be at risk of cardiac stress and/or skeletal muscle stress. Another aspect of the present invention relates to the methods, systems and computer readable media detect a level of Fstl expression, such as Fstl1 polypeptide or mRNA expression in a biological sample obtained from a subject where a low level relative to a reference Fstl expression level is indicative of a subject having, or is at risk of diabetes and/or metabolic dysfunction. In such embodiments, the method futher comprises administering or undertaking an appropriate therapy in a subject identified to have or be at risk of diabetes and/or metabolic dysfunction.

### CROSS REFERENCE TO RELATED APPLICATION

This application claims benefit under 35 U.S.C. §111 of the U.S. Provisional applications No. 61/057,575 filed May 30, 2008, the contents of which are incorporated herein by reference in their entirety.

### FIELD OF THE INVENTION

The present invention relates to the field of diagnostic agents in diagnosis of heart related disorders, and in skeletal muscle injury. It also relates to the field of diagnostic agents for metabolic health.

### BACKGROUND

New diagnostic agents are required to improve our diagnosis of cardiac and skeletal muscle disease (R. E. Gerzten 2008 Nature Insight 451:949). While markers for cardiac disease have been identified (e.g. troponin I and T and creatine kinase), we have little knowledge of circulating proteins that could be prognostic or diagnostic for various muscle diseases including muscular dystrophies, muscle atrophy (cardiac and cancer cachexia), sarcopenia, inclusion body myocytis.

LAD ligation (left anterior descending coronary artery ligation) is a procedure to generate a rat model of heart failure. Ligation of the LAD occludes supply of oxygen and nutrients to the heart muscle, approximating the events of a “heart attack” in humans. Such occlusion results in weakening of the muscle, changes to the overall structure of the heart and cardiac dysfunction. As this model closely mimics heart failure disease progression in humans, it has proven to be a valuable model for the investigation of the mechanisms of heart failure and for the preclinical testing of pharmacological therapies, some of which are now in use in humans. It is also useful in identification of diagnostic and prognostic agents.

### SUMMARY OF THE INVENTION

In one respect, the present invention relates to in part, a method and system for detecting cardiac stress, and/or injury or both in an individual. In one embodiment, the method comprises determining the level of Fstl-1 in a biological sample of the individual, and comparing the determined level of Fstl-1 with a comparably obtained prior level of Fstl-1 in the individual, wherein an elevation in the determined level of at least about 30%, as compared to the prior level, indicates cardiac stress, and/or injury or both in the individual.

In another respect, the present invention relates to a method for assessing an individual for cardiac stress, and/or injury or both. The method comprises determining the relative level of Fstl-1 in a biological sample of the individual, wherein an increased relative level of Fstl-1 in the individual indicates cardiac stress in the individual.

In another respect, the present invention relates to a method for determining prognosis of a subject following treatment of cardiac injury. The method comprises determining the level of Fstl-1 in a biological sample of the individual, and comparing the determined level of Fstl-1 with a comparably obtained prior level of Fstl-1 in the individual, wherein a reduction in the determined level of at least about 30% as compared to the prior level, indicates likely recovery, wherein no change in the level indicates stabilization of the subject, and wherein an elevation in the determined level by at least about 30%, as compared to the prior level, indicates an absence of recovery.

With respect to the methods disclosed regarding cardiac stress, and/or injury or both, the cardiac injury may be a myocardial infarction.

In another respect, the present invention relates to a method for determining whether an individual is at risk for cardiac injury. The method comprises determining the relative level of Fstl-1 in a biological sample of the individual, wherein an increased relative level of Fstl-1 in the individual indicates cardiac stress in the individual.

In another respect, the present invention relates to a method for determining the relative level of Fstl1 in an individual, as it relates to cardiac stress and/or injury in the individual. The method comprises determining the amount of Fstl1 in a biological sample from the individual and comparing the determined amount of Fstl1 to an appropriate control.

In one embodiment of the methods, the relative level of Fstl1 in the individual is determined by determining the level of Fstl-1 in a biological sample of the individual, and comparing the determined level of Fstl-1 with a comparably obtained prior level of Fstl-1 in the individual. An elevation in the determined level of at least about 30%, as compared to the prior level, indicates an increased relative level, and reduction in the determined level of at least about 30% indicates a decreased relative level.

In one embodiment of the methods, the method further comprises correlating the determination to a scale of cardiac injury to thereby determine the likelihood of cardiac injury in the individual. In another embodiment, the amount of Fstl-1 is determined by quantitative detection of Fstl-1 protein/polypeptide. In one embodiment, the detection of Fstl1 protein or polypeptide is by immuno assay. In one embodiment, the immuno assay is Western blot analysis or ELISA. The biological sample may be blood, serum, plasma, sputum, saliva, stool, tissue, urine, stool, lymph fluid, tears, or milk. The tissue may be from an organ, skeletal muscle, or neuronal tissue. The tissue may be cardiac tissue. The tissue may be obtained from a biopsy. Fst-1 levels can be determined through quantitative detection of Fst-1 mRNA.

In one embodiment of the methods, the subject has experienced one or more symptoms or risk factors for one or more of pressure overload hypertrophy, myocardial infarction, angina pectoris (stable and unstable), cardiomyopathy, myocarditis, congestive heart failure, reinfarction, atherosclerosis, acute coronary syndrome, idiopathic cardiomyopathy, drug-induced cardiomyopathy, alcoholic cardiomyopathy, hypertensive cardiomyopathy, atheromatous disorders of the major blood vessels, metabolic cardiomyopathy, diabetic cardiomyopathy. Ischemic cardiomyopathy, hypertensive cardiomyopathy, dilated cardiomyopathy, hypertrophic cardiomyopathy, arrhythmogenic right ventricular cardiomyopathy, left ventricular hypertrophy, cardiac arrhythmias, and restrictive cardiomyopathy inflammatory cardiomyopathy. In another embodiment, the biological sample is taken following administration of a cardioprotective agent.

Cardioprotective agents can be an anti-inflammatory agent, anti-thrombotic agent and/or fibrinolytic agent, anti-platelet agent, lipid reducing agent, direct thrombin inhibitor, and glycoprotein IIb/IIIa receptor inhibitor, agent that binds to cellular adhesion molecules and inhibits the ability of white blood cells to attach to such molecules (e.g. anti-cellular adhesion molecule antibodies), and combinations thereof. The cardioprotective agent may increase the subject's Fstl-1. In one embodiment, the method may further comprise determining the level of an additional agent in a biological sample of the individual. The additional agent can be a marker of cardiac stress, and/or injury or both. The Fstl-1 expression level and the additional agent may be determined from the same biological sample.

In another respect, the present invention relates to a method for detecting skeletal muscle stress and/or injury in an individual. The method comprises determining the level of Fstl-1 expression in a biological sample of the individual, and comparing the determined level of Fstl-1 expression with a comparably obtained prior level of Fstl-1 expression in the individual. An elevation in the determined level of at least about 30%, as compared to the prior level, indicates skeletal muscle stress and/or injury in the individual.

In another respect, the present invention relates to a method for assessing an individual for skeletal muscle stress and/or injury, comprising determining the relative level of Fstl-1 in a biological sample of the individual, wherein an increased relative level of Fstl-1 in the individual indicates skeletal muscle stress and/or injury in the individual.

In another respect, the present invention relates to a method for determining the relative level of Fstl1 in an individual, as it relates to skeletal muscle stress and/or injury in the individual. The method comprises determining the amount of Fstl1 in a biological sample from the individual and comparing the determined level of Fstl1 to an appropriate control.

In one embodiment of the methods, the relative level of Fstl1 in the individual is determined by determining the level of Fstl-1 in a biological sample of the individual, and comparing the determined level of Fstl-1 with a comparably obtained prior level of Fstl-1 in the individual. An elevation in the determined level of at least about 30%, as compared to the prior level, indicates an increased relative level, and reduction in the determined level of at least about 30% indicates a decreased relative level.

In another embodiment of the methods, the method further comprises correlating the determination to a scale of skeletal muscle injury for one or more ischemic muscle diseases to thereby track the onset and/or development of an ischemic muscle disease in the individual. The individual may suffer from or be at risk for a disease that results from or produces skeletal muscle ischemia. The muscle disease can be muscular dystrophy, muscle atrophy, sarcopenia, or inclusion body myocytis. In one embodiment, the amount of Fstl-1 is determined by quantitative detection of Fstl-1 protein/polypeptide. Detection of Fstl-1 protein/polypeptide can be by immuno assay such as Western blot analysis or ELISA. The biological sample can be blood, serum, plasma, sputum, saliva, stool, tissue, urine, lymph fluid, tears and milk. In one embodiment, the tissue is skeletal muscle tissue. In one embodiment, the biological sample is tissue. The tissue can be obtained from a biopsy.

In one embodiment of the invention, the subject has experienced one or more symptoms or risk factors for one or more of muscular dystrophy, muscle atrophy, sarcopenia, inclusion body myocytis. In one embodiment, the biological sample is taken following administration of a therapeutic agent that increases the subject's Fstl-1.

In another respect, the present invention relates to a method of determining the likelihood of development and/or progression of diabetes in an individual. The method comprises detecting the relative level of Fstl-1 in a biological sample of the individual. An increased level of Fstl-1indicates a decreased likelihood of development and/or progression of diabetes, and a decreased level of Fstl-1 indicates an increased likelihood of development and/or progression of diabetes.

In one embodiment, the individual is at risk for diabetes. In one embodiment, the individual undergoes treatment or therapy for diabetes or for the prevention of diabetes.

In another respect, the present invention relates to a method for detecting metabolic dysfunction in an individual. The method comprises detecting the relative level of Fstl-1 in a biological sample of the individual. A decreased level of Fstl-1 indicates a metabolic dysfunction in the individual. In one embodiment the biological sample is skeletal muscle.

One aspect of the present invention relates to a system for analyzing a biological sample from a subject, where the system comprises: (a) a determination module configured to receive a biological sample and to determine Fstl1 expression level information, wherein the Fstl1 expression level information, comprises: (i) the Fstl1 polypeptide expression level in the biological sample; and/or (ii) the Fstl1 gene expression level in the biological sample; (b) a connection from the determination module to transmit the Fstl1 expression level information to an electronic computer, wherein the computer comprises a storage device, a comparison module and a display module; (c) the storage device configured to store Fstl1 expression level information from the determination module; (d) the comparison module adapted to compare the Fstl1 expression level information stored on the storage device with reference data, and to provide a comparison result, wherein the comparison result comprises; (i) a comparison of the Fstl1 expression level in the biological sample with the reference Fstl1 expression level, and (ii) a determination of the Fstl1 expression level in the biological sample above or below a threshold level relative to the reference Fstl1 expression level, wherein a Fstl1 expression level above the threshold level is indicative of cardiac stress and/or skeletal muscle stress; and wherein a Fstl1 expression level below the threshold level is indicative of diabetes; and/or metabolic disorder; (e) the display module for displaying a content based in part on the comparison result for the user, wherein the content is a signal indicative of the likelihood of at least one of: cardiac stress and/or skeletal muscle stress and/or diabetes; and/or a metabolic disorder in the subject.

Another aspect of the present invention relates to a computer readable medium having computer readable instructions recorded thereon to define software modules including a comparison module and a display module for implementing a method on a computer, said method comprising: (a) comparing with the comparison module the data stored on a storage device with reference data to provide a comparison result, wherein the comparison result is the Fstl1 expression level in the biological above a threshold level relative to a reference Fstl1 expression level that is indicative of cardiac stress or skeletal muscle stress; and (b) displaying a content based in part on the comparison result for the user, wherein the content is a signal indicative of cardiac stress and/or skeletal muscle stress.

In some embodiments, the threshold level in the system, computer-readable medium and methods as disclosed herein that is indicative of cardiac stress or skeletal muscle stress is at a level of at least about 30% above the control or reference Fstl1 expression level, for example, where the level of Fstl1 expression in the biological sample is at least 30% above the reference Fstl1 expression level, is indicative of a subject likely to have or be at risk of cardiac stress and/or skeletal muscle stress. In some embodiments the threshold is at least 35%, or at least 40% or at least 45% or above 45%, for example where the level of Fstl1 expression in the biological sample is at least 35%, or at least 40% or at least 45% or above 45% above the reference Fstl1 expression level, it is indicative of a subject likely to have or be at risk of cardiac stress and/or skeletal muscle stress.

In some embodiments, the threshold level in the system, computer-readable media and methods as disclosed herein that is indicative of diabetes and/or metabolic dysfunction is at least 30%, for example, where the level of Fstl1 expression in the biological sample is at least 30% below the reference Fstl1 expression level, it is indicative of a subject likely to have or be at risk of diabetes and/or metabolic dysfunction. In some embodiments the threshold is at least 35% below, or at least 40% below or at least 45% below or more than 45% below, for example where the level of Fstl1 expression in the biological sample is at least 35%, or at least 40% or at least 45% or above 45% below the reference Fstl1 expression level, it is indicative of a subject likely to have or be at risk of diabetes and/or metabolic dysfunction.

In some embodiments, the system, computer-readable media and methods as disclosed herein is used to measure the Fstl1 expression level in a biological sample, where the Fstl1 expression level is the Fstl1 polypeptide expression level, for example, the expression level of the polypeptide of SEQ ID NO: 1. In some embodiments, the Fstl1 polypeptide expression level is measured by immuno assay, for example western blot analysis or ELISA, or a highthrough-put protein detection method, for example but are not limited to automated immunohistochemistry apparatus, for example, robotically automated immunohistochemistry apparatus which in an automated system section the tissue or biological sample specimen, prepare slides, perform immunohistochemistry procedure and detect intensity of immunostaining, such as intensity of anti-Fstl1 antibody staining in the biological sample or tissue and produce output data. Examples of such automated immunohistochemistry apparatus are commercially available, for example such Autostainers 360, 480, 720 and Labvision PT module machines from LabVision Corporation, which are disclosed in U.S. Pat. Nos. 7,435,383; 6,998,270; 6,746,851, 6,735,531; 6,349,264; and 5,839,091 which are incorporated herein in their entirety by reference. Other commercially available automated immunohistochemistry instruments are also encompassed for use in the present invention, for example, but not are limited BOND™ Automated Immunohistochemistry & In Situ Hybridization System, Automate slide loader from GTI vision. Automated analysis of immunohistochemistry can be performed by commercially available systems such as, for example, IHC Scorer and Path EX, which can be combined with the Applied spectral Images (ASI) CytoLab view, also available from GTI vision or Applied Spectral Imaging (ASI) which can all be integrated into data sharing systems such as, for example, Laboratory Information System (LIS), which incorporates Picture Archive Communication System (PACS), also available from Applied Spectral Imaging (ASI) (see world-wide-web: spectral-imaging.com). Other a determination module can be an automated immunohistochemistry systems such as NexES® automated immunohistochemistry (IHC) slide staining system or BenchMark® LT automated IHC instrument from Ventana Discovery SA, which can be combined with VIAS™ image analysis system also available Ventana Discovery. BioGenex Super Sensitive MultiLink® Detection Systems, in either manual or automated protocols can also be used as the detection module, preferably using the BioGenex Automated Staining Systems. Such systems can be combined with a BioGenex automated staining systems, the i6000™ (and its predecessor, the OptiMax® Plus), which is geared for the Clinical Diagnostics lab, and the GenoMx 6000™, for Drug Discovery labs. Both systems BioGenex systems perform “All-in-One, All-at-Once” functions for cell and tissue testing, such as Immunohistochemistry (IHC) and In Situ Hybridization (ISH).

As an example, a determination module used in the system, computer-readable media and methods as disclosed herein for determining Fstl1 expression level measures the level of Fstl1 polypeptide expression, for instance the determination module is configured to detect the total level of Fstl1 polypeptide amount using any known systems for automated protein expression analysis, including for example, but not limited Mass Spectrometry systems including MALDI-TOF, or Matrix Assisted Laser Desorption Ionization—Time of Flight systems; SELDI-TOF-MS ProteinChip array profiling systems, e.g. Machines with Ciphergen Protein Biology System II™ software; systems for analyzing gene expression data (see for example U.S. 2003/0194711); systems for array based expression analysis, for example HT array systems and cartridge array systems available from Affymetrix (Santa Clara, Calif. 95051) AutoLoader, Complete GeneChip® Instrument System, Fluidics Station 450, Hybridization Oven 645, QC Toolbox Software Kit, Scanner 3000 7G, Scanner 3000 7G plus Targeted Genotyping System, Scanner 3000 7G Whole-Genome Association System, GeneTitan™ Instrument, GeneChip® Array Station, HT Array; an automated ELISA system (e.g. DSX® or DS2® form Dynax, Chantilly, Va. or the ENEASYSTEM III®, Triturus®, The Mago® Plus); Densitometers (e.g. X-Rite-508-Spectro Densitometer®, The HYRYS™ 2 densitometer); automated Fluorescence insitu hybridization systems (see for example, U.S. Pat. No. 6,136,540); 2D gel imaging systems coupled with 2-D imaging software; microplate readers; Fluorescence activated cell sorters (FACS) (e.g. Flow Cytometer FACSVantage SE, Becton Dickinson); radio isotope analyzers (e.g. scintillation counters).

In some embodiments, the Fstl1 expression level is the Fstl1 gene expression level, for example the expression level of the nucleotide of SEQ ID NO: 2 or expression level of Fstl1 mRNA.

In some embodiments, the system, computer-readable media and methods as disclosed herein is used to measure the Fstl1 expression level in the biological sample selected from, for example, but not limited to, a muscle sample, a tissue sample, a biopsy sample, an ex vivo cultivated sample, a ex vivo cultivated tissue sample, a surgically dissected tissue sample, a blood sample, a plasma sample, a lymph fluid sample, a primary ascite sample, a serum sample, a sputum sample, a saliva sample, a stool sample, a urine sample, a lymph fluid sample, a tear sample, a milk sample. In some embodiments, a tissue sample is from an organ, skeletal muscle, neuronal tissue. In some embodiments, the tissue sample is a cardiac tissue sample or a skeletal muscle tissue sample, for example, a biopsy cardiac tissue sample or biopsy skeletal muscle tissue sample.

In some embodiments, the system, computer-readable media and methods as disclosed herein is used to measure the Fstl1 expression level in biological sample is obtained from a mammalian subject, for example a human subject. In some embodiments, the subject has had a prior injury, for example, but not limited to, a cardiac injury such as, for example a myocardial infarction.

In some embodiments, the system, computer-readable media and methods as disclosed herein is used to measure the Fstl1 expression level in biological sample obtained from a subject who has experienced one or more symptoms or risk factors for one or more of pressure overload hypertrophy, myocardial infarction, angina pectoris (stable and unstable), cardiomyopathy, myocarditis, congestive heart failure, reinfarction, atherosclerosis, acute coronary syndrome, idiopathic cardiomyopathy, drug-induced cardiomyopathy, alcoholic cardiomyopathy, hypertensive cardiomyopathy, atheromatous disorders of the major blood vessels, metabolic cardiomyopathy, diabetic cardiomyopathy. Ischemic cardiomyopathy, hypertensive cardiomyopathy, dilated cardiomyopathy, hypertrophic cardiomyopathy, arrhythmogenic right ventricular cardiomyopathy, left ventricular hypertrophy, cardiac arrhythmias. restrictive cardiomyopathy inflammatory cardiomyopathy.

In some embodiments, the system, computer-readable media and methods as disclosed herein is used to measure the Fstl1 expression level in biological sample obtained from a subject following the administration of a cardioprotective agent, for example, any cardioprotective agent as disclosed herein, or a cardioprotective agent which increases the expression level of Fstl1 in a biological sample obtained from the subject.

In some embodiments, the system, computer-readable media and methods as disclosed herein comprises a determination module which has been configured to determine the level of an additional agent in the biological sample, for example, a marker of cardiac stress, and/or injury or both.

In some embodiments, the system, computer-readable media and methods as disclosed herein is used to measure the Fstl1 expression level in biological sample to indicate the if a subject is a at risk of skeletal muscle stress, for example skeletal muscle distress, such as ischemic muscle disease or ischemic injury. Accordingly, in some embodiments, the system, computer-readable media and methods as disclosed herein is used to identify if a subject is at risk of developing, or has a skeletal muscle ischemia disease. In some embodiments, the skeletal muscle ischemia disease is, for example, but not limited to, muscular dystrophy, muscle atrophy, sarcopenia, inclusion body myocytis, and other skeletal muscle diseases.

In some embodiments, the system, computer-readable media and methods as disclosed herein is used to measure the Fstl1 expression level in biological sample obtained from a subject, where the biological sample is selected from the group consisting of: a muscle sample, a tissue sample, a biopsy sample, an ex vivo cultivated sample, a ex vivo cultivated tissue sample, a surgically dissected tissue.

Another aspect of the present invention relates to a method of treating a subject at risk for cardiac stress and/or skeletal muscle stress and/or diabetes and/or metabolic dysfunction comprising; (a) determining if the subject is at risk for cardiac stress and/or skeletal muscle stress and/or diabetes and/or metabolic dysfunction by measuring Fstl1 expression level in a biological sample obtained from the subject, and if high levels (e.g. at least about 30% above a reference Fstl1 expression level) of Fstl1 polypeptide expression or Fstl1 gene expression or Fstl1 mRNA expression in the biological sample from the subject is detected, it indicates that the subject is likely to be at risk for cardiac stress and/or skeletal muscle stress, whereas if low levels (e.g. at least about 30% below a reference Fstl1 expression level) of Fstl1 polypeptide expression or Fstl1 gene expression or Fstl1 mRNA expression in the biological sample indicates the subject is likely to be at risk for diabetes and/or metabolic dysfunction, and (b) administering to a subject determined to be at risk for cardiac stress and/or skeletal muscle stress a cardioprotective drug or skeletal muscle stress drug, or administering to a subject determined to be at risk for diabetes and/or metabolic dysfunction an appropriate therapy.

In some embodiments, a subject identified to be at risk for skeletal muscle stress is administered a skeletal muscle stress drug, for example, a nutritional program, or exercise program or other skeletal muscle stress therapy for regeneration of muscle which is known by a person of ordinary skill in the art.

### BRIEF DESCRIPTION OF THE DRAWINGS

FIGS. 1A-1F show upregulation of Fstl1 by pathological stimuli in the heart. FIGS. 1A-1C shows QRT-PCR analysis of Fstl1 transcript expression of Fstl1 mRNA after (1A) transverse aortic constriction (TAC) (1B) ischemia/reperfusion (I/R) injury and (1C) myocardial infarction (MI) resulting from permanent LAD ligation. The data are normalized to the intensity of the GAPDH signal, and are compared to Fstl1 transcript expression level in sham-operated heart. n=3-6. *P<0.05 vs. sham. FIG. 1D shows western immunoblot analysis of Fstl1 expression in mouse heart lysate and serum after sham operation (left 2 lanes) and myocardial infarction (right 4 lanes). FIG. 1E shows quantitation of Fstl1 protein expression in hearts of mice subjected to sham surgery or myocardial infarction. FIG. 1F shows quantitation of Fstl1 protein levels in serum of mice subjected to sham surgery or myocardial infarction.

FIG. 2A-2B show upregulation of Fstl1 in Akt-activated heart. FIG. 2A shows western blots from transgenic mouse heart lysate. Transgene (myr-Akt tagged with HA) induction was confirmed by probing with antibody against HA (middle panel). Two weeks after myr-Akt transgene induction (lane 3, 4 and 5), Fstl1 expression level was upregulated (upper panel) compared to control mice that undergo the same Dox administration/withdrawal protocol (lanes 1 and 2). Each lane represents a difference transgenic or control mouse. FIG. 2B shows quantification of the intensity of the bands for Fstl1 normalized with that for alpha-tubulin. n=3-4. *P<0.05.

FIGS. 3A-3B show Fstl1 secreted from cells. FIG. 3A shows HEK293 cells were transfected with a plasmid expressing V5 epitope-tagged Fstl1 protein. Western blot analysis using anti-V5 antibody indicates that Fstl1 is present in the cell pellet and the culture media. FIG. 3B shows NRVMs were transfected with an adenovirus expressing Fstl1 or β-galactosidase. Western blot analysis using anti-Fstl1 antibody revealed Fstl1 protein expression in the cell pellet and the culture media, and the intensity of these bands increased in cells that were transduced with Ad-Fstl1.

FIG. 4 shows activation of intracellular signaling pathways by Fstl1. After transfection of Ad-Fstl1, NRVMs were cultured for 36 hours and cell lysates were prepared for Western blot analysis. Transduction of Ad-Fstl1 led to the activating phosphorylation of Akt1 and 2 and ERK1/2 in NRVMs. Phosphorylation of FOXO1 and 3 and mTOR, downstream targets of Akt signaling, also increased.

FIGS. 5A-5B shows Fstl1 inhibits hypoxia/reoxygenation (H/R)-induced apoptosis. FIG. 5A shows quantification of TUNEL-positive cell number of tunnel Serum-deprived NRVMs following 12 hours of hypoxia followed by 24 hours of reoxygenation (H/R). Cells were stained with TUNEL to identify apoptotic nuclei and DAPI to identify total nuclei (data not shown). Cultures were transfected with Ad-Fstl1 or Ad-βgal prior to H/R. *P<0.01 compared to Ad-βgal transfected NRVMs after H/R. FIG. 5B shows nucleosome fragmentation ELISA shows that transduction with Ad-Fstl1 reduced apoptosis after H/R. *P<0.01 compared to Ad-βgal transfected NRVMs after H/R.

FIGS. 6A-5B shows Fstl1-mediated cytoprotection is mitigated by PI3K or MEK1 inhibition. FIG. 6A shows quantification of TUNEL-positive cells under different culture conditions. Adenovirus-transduced NRVMs were stained with TUNEL and DAPI (data not shown). Three hours prior to the induction of hypoxia, NRVMs were pretreated with a PI3K inhibitor (LY294002; LY, 10 μmol/L), a MEK1 inhibitor (U0126; U, 10 μmol/L) or vehicle. Apoptotic nuclei were identified by TUNEL staining. *P<0.05. FIG. 6B shows nucleosome fragmentation ELISA showed that Fstl1-mediated protection of myocytes from apoptosis was reversed by treatment with the PI3K inhibitor or the MEKI inhibitor. *P<0.05.

FIGS. 7A-7D show knock-down of endogenous Fstl1 increases apoptosis in response to hypoxia/reoxygenation (H/R). FIG. 7A shows QRT-PCR analysis indicates that transduction of siRNA decreased Fstl1 mRNA by approximately 70% (*P<0.05) in cultured cardiac myocytes. FIG. 7B shows representative immunoblot analysis of Fstl1 protein expression in cell lysates and culture media of cardiac myocyte cultures treated with siRNA directed to Fstl1 or an unrelated sequence. FIG. 7C shows nucleosome fragmentation assay shows that knock-down of endogenous Fstl1 expression in serum-deprived cardiac myocyte cultures increases apoptosis in cells under normoxic conditions and in cells treated with H/R (P<0.05). FIG. 7D shows immunoblot analysis reveals that Fstl1 ablation results in decreased Akt phosphorylation, but not that of ERK, in cultured cardiac myocytes. Two commercial sources of siRNA targeting Fstl1 were employed in these assays.

FIGS. 8A-C show Fstl1 protects against myocardial ischemia/reperfusion (I/R) injury in vivo. FIG. 8A shows western blot analysis of serum proteins from mice following the intravenous delivery of the indicated adenoviral vector. Sera was collected and analyzed 5 days after adenovirus delivery. Serum Fstl1 level was markedly increased by administration of Ad-Fstl1. Heart sections were stained with Evans blue and TTC to detect the infarction zone resulting from 30 minutes ischemia and 24 hour reperfusion (data not shown). I/R injury was performed 5 days after adenoviral injection. FIG. 8B shows quantification of infarction size of each experimental group (n=10 for Ad-βgal group, n=7 for Ad-Fstl1 group). *P<0.05 compared to Ad-βgal injected group. Heart sections stained with TUNEL, sarcomeric actin and DAPI (data not shown) which was used for quantification of TUNEL positive myocyte number, shown in FIG. 8C that administration of Ad-Fstl1 decreased apoptosis (n=3 for each group). *P<0.01 compared to Ad-βgal injected group.

FIGS. 9A-9C show transgenic Akt activation increases capillary density and Fstl1 expression in gastrocnemius muscle. FIG. 9A shows increased capillary vessels in gastrocnemius muscles in muscle-specific Akt-TG mice following 2 weeks of Akt1 activation. Immunostaining of gastrocnemius tissues of control (n=5) and Akt-TG (n=5) mice was performed with anti-CD31 monoclonal antibody. FIG. 9B shows quantification of capillary density expressed as the number of capillaries per high power field. Results are shown as the mean±SEM. *p<0.01 vs. Control mice. FIG. 9C shows quantatative analysis by RT-PCR of upregulation of Fstl1 expression in gastrocnemius muscle in muscle-specific Akt-TG mice after Akt1 activation for 2 weeks. Fstl1 expression was determined by QRT-PCR (shown in FIG. 9C) and Western blot analyses, shown in FIG. 9D (n=5). Fstl1 mRNA levels were expressed relative to levels of GAPDH mRNA. Results are expressed relative to control. Results are shown as the mean±SEM. *p<0.01 vs. Control. FIG. 9E shows Fstl1 is secreted from C2C12 myotube cultures. C2C12 cells were transduced with adenoviral vectors expressing Fstl1 (Ad-Fstl1), or β-galactosidase (Ad-β-gal) for 16 h followed by 24 h of incubation in serum-free media. Fstl1 protein levels were determined in media and cell lysates by Western blot analysis.

FIGS. 10A-10B show elevated Fstl1 levels in ischemic muscle and serum after hindlimb ischemic surgery. FIG. 10A shows QRT-PCR analysis of Fstl1 expression in non-ischemic or ischemic adductor muscles (n=5) at day 7 after femoral artery resection. Fstl1 transcript levels were expressed relative to levels of GAPDH mRNA. Results are expressed relative to control. FIG. 10B shows a western blot of Fstl1 expression in non-ischemic and ischemic skeletal muscle. Serum was collected from control or mice subjected to hindlimb ischemic surgery, and Fstl1 levels were determined by Western blot analyses. Results are shown as the mean±SEM. *p<0.05 vs. Non-ischemic.

FIGS. 11A-11C shows Fstl1 promotes the perfusion recovery and capillary vessel formation of ischemic limbs in mice in vivo. Adenoviral vectors expressing Fstl1 (Ad-Fstl1), or β-galactosidase (Ad-β-gal, Control) were injected into 5 sites in adductor muscle of wild-type mice (2×10⁸ pfu each) at 3 days prior to ischemic surgery. FIG. 11A shows a western blot analysis of Fstl1 expression in ischemic muscle at 6 days after injection of Ad-Fstl1 or Ad-β-gal. Fstl1 protein expression. Representative blots are shown. FIG. 11B shows quantitative analysis of the ischemic/nonischemic LDBF ratio in wild-type mice treated with Ad-Fstl1 (n=8) and Ad-β-gal (n=8). Results are shown as the mean±SD. *p<0.05 vs. Control mice. **p<0.01 vs. Control mice. FIG. 11C shows quantitative analysis of capillary density in ischemic muscles of wild-type mice treated with Ad-Fstl1 (n=5) and Ad-β-gal (n=5) on postoperative day 14. Immunostaining of ischemic tissues was performed with anti-CD31 monoclonal antibody (upper panel, FIG. 11C), and capillary density was quantified, as shown in bottom panel FIG. 11C, as the number of capillaries per muscle fiber. Results are shown as the mean±SEM. *p<0.01 vs. Control mice.

FIGS. 12A-12C show Fstl1 promotes endothelial cell migration and differentiation into vascular-like structures. FIG. 12A shows a western blot analysis of the expression of Fstl1 protein in media and cell lysates from HUVECs. HUVECs were transduced with Ad-Fstl1 and Ad-β-gal for 8 h followed by 24 h of incubation in serum-free media. Fstl1 protein levels were determined in media and cell lysates from HUVECs. Representative blots are shown. FIG. 12B shows endothelial cell network formation in response to Fstl1. After 24 h of serum-deprivation and transduction with Ad-Fstl1 and Ad-β-gal, HUVECs were seeded on Matrigel-coated culture dishes (data not shown) and the network formation was quantified. FIG. 12C shows migratory activities of HUVECs following treatment with Fstl1. A modified Boyden chamber assay was performed using HUVECs transduced with Ad-Fstl1 and Ad-β-gal. Results are shown as the mean±SEM (n=7-8). Results are expressed relative to the values compared to control. *p<0.01 vs. Ad-β-gal.

FIGS. 13A-13C shows Fstl1 protects endothelial cells from apoptosis. HUVECs were transduced with Ad-Fstl1 and Ad-β-gal for 8 h followed by incubation with serum-free media for 48 h. FIG. 13A shows an inhibitory effect of Fstl1 on Nucleosome fragmentation of HUVECs. Nucleosome fragmentation was assessed by ELISA. Results are expressed relative to the values compared to control. FIG. 13B shows inhibition of HUVEC death by Fstl1 assessed by a quantitative MTS-based assay. FIG. 13C shows quantitative analysis of the frequency of TUNEL-positive HUVECs reduction after treatment with Fstl1. Apoptotic nuclei were identified by TUNEL staining (green), and total nuclei were identified by DAPI counterstaining (blue) (data not shown). Results are shown as the mean±SEM (n=8-10). *p<0.01 vs. Ad-β-gal.

FIGS. 14A-14B show Fstl1-mediated angiogenic and survival responses are dependent on Akt signaling. FIG. 14A show western blot analysis of Fstl1-stimulated signaling in endothelial cells. HUVECs were transduced with Ad-Fstl1 and Ad-β-gal for 8 h followed by 24 h of incubation with serum-free media. Changes in the phosphorylation of eNOS (P-eNOS), Akt (P-Akt), GSK (P-GSK) and ERK (P-ERK) following Ad-Fstl1 treatment were determined by Western blot analysis. Representative blots are shown. FIG. 14B shows the role of Akt in regulation of Fstl1-induced signaling. HUVECs were infected with adenoviral constructs encoding dominant-negative Akt1 (Ad-dnAkt) or Ad-βgal at a MOI of 10 along with Ad-Fstl1 or Ad-β-gal at a MOI of 10 for 8 h, followed by serum-deprivation for 24 h. Phosphorylation of eNOS (P-eNOS) and Akt (P-Akt) were determined by Western blot analysis. Representative blots are shown. FIG. 14C and FIG. 14D show the contribution of Akt to Fstl1-mediated angiogenic cellular responses. HUVECs were transduced with Ad-dnAkt or Ad-βgal along with Ad-Fstl1 or Ad-β-gal for 8 h. After 24 h of serum-deprivation, Matrigel (FIG. 14C) or modified Boyden chamber assays (FIG. 14D) were performed. FIG. 14E show the involvement of Akt in Fstl1-induced endothelial cell survival. After transduction with Ad-dnAkt or Ad-βgal along with Ad-Fstl1 or Ad-β-gal for 8 h, cells were incubated in serum-free media. Nucleosome fragmentation was assessed by ELISA. Results are shown as the mean±SEM (n=6-8). Results are expressed relative to the values compared to control. *, p<0.01.

FIGS. 15A-15 C show PI3-kinase and eNOS signaling is involved in Fstl1-induced angiogenic cellular responses. FIG. 15A shows the effect of LY294002 on Fstl1-induced phosphorylation of eNOS and Akt. HUVECs were treated with LY294002 (10 PM) or vehicle following transduction with Ad-Fstl1 or Ad-β-gal. After 24 h serum-deprivation, phosphorylation of eNOS (P-eNOS) and Akt (P-Akt) were determined by Western blot analysis. Representative blots are shown. FIGS. 15B and 15C show western blots of the contribution of PI3-kinase to Fstl1-mediated endothelial differentiation and migration. HUVECs were treated with LY294002 (10 μM), L-NAME (1 mg/ml) or vehicle along with Ad-Fstl1 or Ad-β-gal for 8 h. After 24-h serum-starvation, Matrigel (FIG. 14B) or modified Boyden chamber assays (FIG. 14C) were performed. Results are shown as the mean±SEM (n=5-8). Results are expressed relative to the values compared to control. *, p<0.01.

FIGS. 16A-16C show Fstl1 stimulates ischemia-induced angiogenesis through an eNOS-dependent mechanism. FIG. 16A shows the phosphorylation of eNOS and Akt in ischemic muscle tissues of wild-type and eNOS-KO mice at 6 days after transduction with Ad-Fstl1 or Ad-β-gal. Ad-Fstl1 or Ad-β-gal (Control) was injected into 5 sites in adductor muscle of wild-type and eNOS-KO mice (2×10⁸ pfu each), 3 days before ischemic surgery. Phosphorylation of eNOS (P-eNOS) and Akt (P-Akt), and total eNOS levels were analyzed by Western blotting. Representative blots are shown. FIG. 16B shows Fstl1 expression in ischemic muscle of eNOS-KO mice at 6 days after injection of Ad-Fstl1 or Ad-β-gal. Fstl1 protein expression was determined by Western blot analysis. Representative blots are shown. FIG. 16C shows quantitative analysis of the ischemic/nonischemic LDBF ratio in eNOS-KO mice treated with Ad-Fstl1 (n=7) and Ad-β-gal (n=7). Results are shown as the mean±SD. N.S, not significant.

FIGS. 17A-17B shows Fstl1 transcript expression in skeletal muscle upon aging and in response to fasting. FIG. 17A shows Fstl1 mRNA levels in gastrocnemius muscle in young and aged mice. FIG. 17B shows Fstl1 mRNA levels in gastrocnemius muscle under ad libitum and starved conditions. Fstl1 expression was determined by quantitative real time PCR. Fstl1 mRNA levels were expressed relative to levels of GAPDH mRNA.

FIG. 18 shows decreased Fstl1 levels in skeletal muscle and serum in diabetic ob/ob mice. Fstl1 expression in gastrocnemius muscle in wild-type (WT) and ob/ob mice was measured by Western blot analyses. Serum was collected from WT or ob/ob mice, and Fstl1 levels were determined by Western blot analyses.

FIG. 19 shows a western blot of the detection of Fstl1 in human serum. Serum was collected from a healthy man, and Fstl1 levels were determined under non-denatured and denatured conditions by Western blot analyses. Recombinant Fstl1 protein produced by E-coli was used as a positive control.

FIGS. 20A-20B show association of serum Fstl1 levels with heart failure. Blood was collected from normal subjects and patients with systolic heart failure (depressed left ventricular ejection fraction (LVEF)). Left ventricular mass was calculated by echocardiography. FIG. 20A shows serum Fstl1 levels as determined by semi-quantitative western blot analysis using recombinant Fstl1 protein as a standard protein. FIG. 20B shows a scatter plot of the serum Fstl1 levels with degree of left ventricular mass in individuals with depressed left ventricular ejection.

FIG. 21 is a block diagram showing an example of a system for assessing the Fstl1 expression level in a biological sample and whether a subject has, or is at risk of developing cardiac stress and/or skeletal muscle stress.

FIG. 22 is a block diagram showing exemplary instructions on a computer readable medium for assessing the Fstl1 expression level in a biological sample and whether a subject has, or is at risk of developing cardiac stress and/or skeletal muscle stress (i.e. a positive or negative cardiac stress and/or skeletal muscle stress test result).

### DETAILED DESCRIPTION OF THE INVENTION

The inventors have discovered that measuring changes in Follistatin-like (Fstl1) levels in a subject, such as a human or other individual, can be used to screen for ailments, disorders and susceptibility to certain ailments and disorders. For example, Fstl1 levels are higher in individuals with cardiac or muscle injury. Further, Fstl1 levels are decreased in subjects which exhibit metabolic disorders, for example, diabetes. Thus Fstl1 represents a potential diagnostic target for cardiovascular or skeletal muscle disease or metabolic disorders such as obesity, diabetes and metabolic syndrome.

Follistatin-like 1 (referred to herein as “Fstl1” or “Fstl-1”), also known in the art as FRP and TSC-36, is an extracellular glycoprotein belonging to the BM-40/SPARC/osteonectin family of proteins containing both extracellular calcium-binding and follistatin-like domains. FSTL1 was originally cloned from an osteoblastic cell line as a TGF-β-inducible gene. The protein occurs in two isoforms resulting from differential sialylation. Fstl1 is expressed in skeletal and cardiac muscle and exerts cardioprotective and metabolic actions when administered to mice. Fstl1 transcript levels in skeletal muscle are downregulated upon aging and in fasted mice, and Fstl1 protein levels in serum are downregulated in leptin-deficient ob/ob mice that are obese and diabetic.

One activity of Fstl1 is inhibition of apoptosis. Without being bound by theory, it is thought that Fstl1 is released from certain tissue in response to stress which would necessitate increased angiogenesis for maintenance or repair. As tissue is strained or stressed, Fstl1 levels are increased. As the stress mounts, the tissue becomes further damaged, and increased levels of Fstl1 are produced. The higher levels of damage result in a more apparent or detectable injury. Since Fstl1 is a secreted protein, detectable increases in Fstl1 levels are present in the injured or stressed tissue as well as surrounding tissue, organs, and circulatory and lymphatic systems. As such, levels of Fstl1, in the tissue as well as released from the tissue, can be exploited as a diagnostic indicator of the tissue stress which engenders its production and release. The levels of Fstl1 can also serve as an indicator of metabolic activity, wherein higher levels of Fstl1 correlate with higher levels of tissue strain or stress, and for metabolic activity.

Aspects of the present invention relate to methods for determining whether a person is at risk for, or has suffered a myocardial disorder. An individual can be assessed for cardiac stress, and/or injury or both, by performing methods of detection of cardiac stress, and/or injury or both described herein. One aspect of the present invention is a method for detecting cardiac stress, and/or injury or both in a subject. The method involves determining the Fstl1 level from a relevant biological sample from the subject. Determination of a relatively high level of Fstl1 in the sample of the subject would indicate cardiac stress, and/or injury or both. Determination of a relatively low level of Fstl1 in the sample would indicate absence and/or reduced cardiac stress, and/or injury or both in the subject. As used herein, the term “relatively high” and “relatively low” indicates a significant increase or decrease with respect to an appropriate control.

One such method of determining a relative level of Fstl1 is by comparison to an earlier determined level in the subject (e.g. the control is an earlier obtained sample from the same subject). A significant elevation in the determined Fstl1 level as compared to the prior Fstl1 level in the subject indicates cardiac stress and/or injury in the subject. For example, one can obtain Fstl1 levels as part of annual physicals and changes in such levels from visit to visit can be used to assess an individual's condition.

As used herein, a significant elevation in a determined Fstl1 level as compared to the prior Fstl1 level in the subject refers to a statistically significant elevation by at least about 30% Such a determined increase in Fstl1 is referred to herein as an increased relative level. In one embodiment, the elevation is at least about 35%, 40%, 45%, 50%, 55%, 60%, 65%, 70%, 80%, 100%. The higher the elevation in the level, the more likely the physiological disease risk/development, stress and/or injury occurred to produce the levels. For example, one can look at changes such as 1 fold, 3 fold, 10 fold, 12 fold, etc. In one embodiment, higher levels of elevation would be an indication of higher stress and/or increased injury in the subject.

In another embodiment, a significant reduction in a determined Fstl1 level as compared to the prior Fstl1 level in the subject is also significant. Such changes refer to a statistically significant reduction by at least about 30%. Such a determined decrease in Fstl1 is referred to herein as a decreased relative level. In one embodiment, the reduction is at least about 35%, 40%, 45%, 50%, 55%, 60%, 65%, 70%.

No change in a determined Fstl1 level as compared to the prior Fstl1 level in the subject refers to a situation where there is no statistically significant change in the determined Fstl1 levels. No change in the levels can be used as an indication of stabilization of a subject. Such stabilization results from a lack of progression or from effective therapeutic treatment, and can be an indicator of such.

Preferably the earlier determined level is obtained by like or comparable methods. Comparable methods includes determining the levels from the same or an analogous biological sample. It can also further include determining the levels with the same or analogous assays for Fstl1 quantitation. Increased sensitivity in determining relative Fstl1 levels may be obtained by obtaining the samples from a subject while under similar circumstances (e.g., at the same time of day, at the same amount of time after receipt of therapy, following the same level of exercise, etc.). Relevant parameters with respect to such comparable conditions suitable for obtaining the biological sample and measuring the Fstl1 can be determined by the skilled practitioner.

Another aspect of the invention relates to a method for determining prognosis of a subject following treatment of an injury (e.g. cardiac injury). In one embodiment, the injury is heart failure (e.g. from myocardial infarction). Prognosis is a medical term denoting the skilled physician's prediction of how a patient's disease will progress, and whether there is chance of recovery. The method involves determining the level of Fstl1 in a biological sample of the subject. The determined level is then compared to an earlier obtained level of Fstl1 of the subject, as described above. A significant reduction in the determined level (e.g., by at least about 30%, 35%, 40%, 45%, 50%, 55%, 60%, 65%, 70%) as compared to the prior level, indicates a good prognosis. Such a good prognosis correlates with the likely recovery of the subject. Such a prognosis can be used to indicate that the treatment is effective.

A significant increase in the determined level (e.g., by at least about 30%, 35%, 40%, 45%, 50%, 55%, 60%, 65%, 70%) as compared to the prior level, indicates a poor prognosis. Such a poor prognosis correlates with an absence of recovery. This would indicate that recovery is not progressing. Such a prognosis can be used to indicate that the current treatment is not as effective as desired and may be ineffective. An indication of ineffective treatment can suggest increasing treatment or that alternate treatment methods should be pursued.

A determination of no change in the level of Fstl1 as compared to the prior level can indicate stabilization of the subject. Whether such an indication is positive or negative will depend upon the diagnosis and specific injury. An indication that the condition is not worsening is often held as a positive indication following various heart injuries. Stabilization is often the first goal, to be followed by additional treatments or therapies to further the recovery.

Such determinations of prognosis can be used to determine if the medication given a subject requires adjustment, should stay the same, or be reduced or discontinued. In one embodiment, the cardiac injury is myocardial infarction.

The indication of recovery, stabilization or absence of recovery in the subject can further be correlated with one or more other prognosis indicators determinable in the subject to generate a prognosis with enhanced value.

In some embodiments, the methods and systems as disclosed herein for determining prognosis of a subject following an injury are also suitable for determining prognosis of skeletal muscle injury, whereby the appropriate biological sample is taken and the Fstl1 expression levels determined to reflect Fstl1 levels responsive to such skeletal muscle stress or injury, as described herein.

A detected increased relative level of Fstl1 in a healthy subject, or in a subject without a history of heart disease injury, indicates increased cardiac stress. This stress may precede or correspond with a detectable cardiac injury. Without being bound by theory, stress which precedes such an injury would presumably cause lower increases in relative Fstl1 levels than stress which corresponds with a detectable cardiac injury. Any detectable increase, low or high, would likely be followed up with additional tests to determine the level of stress and/or any detectable injuries. Stress which does not correlate with a detectable cardiac injury would still contribute to an increased risk for cardiac injury. Further, the presence of many detectable cardiac injuries increase the risk for the development of secondary cardiac injuries. As such, another aspect of the invention relates to a method for determining whether a subject is at risk for cardiac injury. The method involves determining the relative level of Fstl1 in a biological sample of the individual, wherein an increased relative level of Fstl1 in the individual indicates cardiac stress that contributes to increased risk of the subject for cardiac injury.

The methods described herein can involve determinations made from apparently healthy individuals. The term “apparently healthy”, as used herein, means individuals who have not previously had or are not aware of a previous adverse cardiovascular event such as a myocardial infarction. Apparently healthy individuals also do not otherwise exhibit symptoms of disease. In other words, such individuals, if examined by a medical professional, would be characterized as healthy and free of symptoms of disease.

In one embodiment, the method is performed on a subject who has experienced or exhibited symptoms of an experienced external stress related to one or more of the following symptoms or risk factors: pressure overload hypertrophy, myocardial infarction, angina pectoris (stable and unstable), cardiomyopathy, myocarditis, congestive heart failure, reinfarction, atherosclerosis, acute coronary syndrome, idiopathic cardiomyopathy, drug-induced cardiomyopathy, alcoholic cardiomyopathy, hypertensive cardiomyopathy, atheromatous disorders of the major blood vessels, metabolic cardiomyopathy, diabetic cardiomyopathy. Ischemic cardiomyopathy, hypertensive cardiomyopathy, dilated cardiomyopathy, hypertrophic cardiomyopathy, arrhythmogenic right ventricular cardiomyopathy, left ventricular hypertrophy, cardiac arrhythmias. restrictive cardiomyopathy, inflammatory cardiomyopathy.

It can be useful to perform the methods described herein on the subject at one or more indicated times following specific experienced symptoms of the subject, such as initial symptoms (e.g., at about 1 hour, 2-5 hours, 10 hours, 12 hours, 24 hours, 36 hours, 48 hours, and/or 72 hours.

The methods described herein which involve detecting and assessing cardiac stress, and/or injury or both can be performed on a subject following administration of one or more cardioprotective agents. As the term is used herein, “cardioprotective agent” refers to an agent which is known or suspected to protect the heart. That is to say, the ingestion of such an agent generally reduces risk of cardiac stress or injury. Such cardioprotective agents are known in the art, and without limitation, include anti-inflammatory agent, anti-thrombotic agent and/or fibrinolytic agent, anti-platelet agent, lipid reducing agent, direct thrombin inhibitor, and glycoprotein IIb/IIIa receptor inhibitor, agent that binds to cellular adhesion molecules and inhibits the ability of white blood cells to attach to such molecules (e.g. anti-cellular adhesion molecule antibodies). In one embodiment, the cardioprotective agent administered increases the subject's Fstl1. One such agent is exogenous Fstl1. Additionally, agents which increase Akt 1 activity are expected to increase Fstl1.

### Skeletal Muscle Embodiments

Fstl1 levels are also increased in tissues in response to skeletal muscle stress and/or injury (e.g. skeletal muscle stress or injury brought on by, or associated with, skeletal muscle ischemia). This Fstl1 is also secreted. As such, aspects of the present invention relate to methods for determining whether a person is at risk for, or has suffered skeletal muscle stress and/or injury. An individual can be assessed for skeletal muscle stress and/or injury by performing methods of detection of skeletal muscle stress and/or injury described herein. One aspect of the present invention is a method for detecting skeletal muscle stress and/or injury in a subject. The method involves determining the Fstl1 level from a relevant biological sample from the subject in relation to an appropriate control.

Determination of a relatively high level of Fstl1 in the sample of the subject would indicate skeletal muscle stress and/or injury. Determination of a relatively low level of Fstl1 in the sample would indicate absence and/or reduced skeletal muscle stress and/or injury in the subject. Fstl1 levels are determined as described herein for the methods involving detection of cardiac stress/injury. Of course, the relevant biological samples, and the relevant controls, will necessarily correlate with the type of stress or injury being assessed. As the term is used herein, a “relevant biological sample” or “relevant sample” is one taken at or near the site of suspected injury or stress, or from a location into which one expects the secreted Fstl1 to migrate. The methods described herein are also useful in detecting ischemia-induced angiogenesis by detecting localized increased levels of Fstl1.

In one embodiment, the method further comprises correlating the determination to a scale of skeletal muscle injury for one or more ischemic muscle diseases known in the art, to track the onset and/or development of an ischemic muscle disease in the individual. This is useful, for example, in an individual who is at risk for, or exhibits early symptoms of a skeletal muscle disease or condition that results from or produces skeletal muscle ischemia. Such disease include, without limitation, muscular dystrophy, muscle atrophy, sarcopenia, inclusion body myocytis, cardiac cachexia, and cancer cachexia. Early symptoms are one or more known or suspected symptoms of the indicated disorder.

Methods that involve analysis of skeletal muscle stress and/or injury can further be used to determine prognosis of a subject following treatment of the injury. Such analysis is comparable to methods described herein for determining cardiac injury (e.g., for treatment or prognosis).

Another aspect of the invention relates to a method for determining the relative level of Fstl1 in an individual, as it relates to skeletal muscle stress and/or injury in the individual, comprising, determining the amount of Fstl1 in a biological sample from the individual and comparing the determined level of Fstl1 to an appropriate control.

In such methods described herein, which relate to skeletal muscle stress or injury, a particularly useful biological sample is skeletal muscle or neuronal tissue.

The methods described herein can further comprise determining the level of an additional agent in a biological sample of the individual (the same sample or a different sample). Particularly useful agents are those that indicate information to the skilled practitioner regarding the condition which is being analyzed. For example, the methods described herein which involve detecting and assessing cardiac stress, and/or injury or both can further comprise assessing one or more agents or factors which serve as another marker of cardiac stress, and/or injury or both, otherwise referred to herein as a marker of cardiac health and/or function. Such markers include, without limitation, cardiac creatine kinase (CK), and Troponin T (TnT). The methods described herein which involve detecting and assessing skeletal muscle stress and/or injury can further comprise assessing one or more agents or factors which serve as another marker of skeletal muscle stress and/or injury, e.g. markers for progression of such skeletal muscle diseases such as muscular dystrophy, muscle atrophy, sarcopenia, and inclusion body myocytis. The methods described herein which involve detecting and assessing metabolic dysfunction (e.g. risk for diabetes or obesity) can further comprise assessing one or more agents which serve as another marker of metabolic dysfunction (e.g. glucose, insulin, etc.).

Other particularly useful agents for analysis are those that indicate information to the skilled practitioner regarding another condition which is being ruled out or taken into consideration with respect to the condition being analyzed. For example, if one is performing analysis on cardiac stress, and/or injury or both, it can be useful to further perform analysis of potential skeletal muscle stress and/or metabolic dysfunction of the subject (e.g. by analysis of a marker or the subject's history or overall condition), in order to accurately correlate the results obtained for cardiac stress/injury with an actual experienced condition. In addition, if one is performing analysis on skeletal muscle stress and/or injury, it can be useful to further perform analysis of potential cardiac stress and/or metabolic dysfunction of the subject. Likewise, if one is performing analysis on metabolic dysfunction, it can be useful to further perform analysis of potential cardiac stress and/or skeletal muscle stress of the subject.

Aspects of the present invention also relate to the use of the levels of Fstl1 to indicate metabolic dysfunction. One aspect of the present invention involves a method for detecting metabolic dysfunction in an individual. The method involves detecting the relative level of Fstl1 in a biological sample of the individual, as described herein. A decreased level of Fstl1, e.g. in comparison to an earlier determined level of Fstl1 when apparently healthy, eating a recommended healthy diet, and getting sufficient exercise, indicates a metabolic dysfunction in the individual. Metabolic dysfunction in an individual can be used to indicate an increased risk of diabetes in the individual. An indication of increased risk can be used to indicate that preventative therapies are warranted. An indication of decreased risk, in a subject who previously had increased risk, can be used to indicate that therapies being used are working.

Another such method is determining the likelihood of development and/or progression of diabetes in an individual. The method involves detecting the relative level of Fstl1 in a biological sample of the individual, as described herein. An increased level of Fstl1 indicates a decreased likelihood of development and/or progression of diabetes, and a decreased level of Fstl1 indicates an increased likelihood of development and/or progression of diabetes.

In one embodiment, the individual is at risk for diabetes due to other symptoms or conditions (e.g. low glucose tolerance, obesity, pregnancy, etc.). In one embodiment, the individual has underwent therapy for diabetes (e.g. insulin administration). The individual may have undergone some form of therapy for prevention of diabetes (e.g. diet modification, an exercise regimen, medication, etc.).

Appropriate biological samples are described herein. In one embodiment, the biological sample is skeletal muscle which has not undergone known stress and/or injury. In another embodiment, the biological sample is blood, lymph, or a blood derived sample (plasma, serum, etc.).

As used herein, the terms “follistatin-like protein 1,” “follistatin-like protein 1 polypeptide” and “Fstl-1” and “Fstl1” are used interchangeably herein, and refer to the human follistatin-like protein 1 polypeptide having an amino acid sequence as in GenBank Accession No. AAH00055, which is incorporated herein by reference. A nucleotide sequence encoding Fstl-1 is at GenBank Accession No. NM007085, which is incorporated herein by reference (see also Table 2, which includes amino acid and nucleotide sequences for Fstl-1, as SEQ ID NOs 1 and 2). The terms also refer to fragments or portions thereof that have one or more signalling, therapeutic or preventive activities of full length Fstl-1, also referred to herein as “protective portions,” as well as to mutants or derivatives of the Fstl-1 polypeptide that retain one or more such activities. By “retain . . . such activity” is meant that a variant or derivative has at least 50% of the subject activity relative to full length, wild-type Fstl-1 polypeptide, preferably at least 60%, 70%, 80%, 90%, 95%, 96%, 97%, 98%, 99% or 100%, or even greater than 100% of an activity relative to the full length wild-type polypeptide. The terms “mutant,” “derivative” or “variant” do not encompass other naturally-occurring follistatin-like proteins, e.g., follistatin, follistatin-like protein 3, etc. Signalling activity encompasses any natural signalling activity, non-limiting examples of which include kinase or phosphatase activity, intracellular calcium regulation, and the ability to associate with and influence the activity of another signalling protein or factor. Signalling activity, however, specifically excludes the ability to provoke an immune response that raises antibodies specific for an Fstl-1 polypeptide.

Follistatin-like protein 1 is also known as FRP and TSC-36. The protein is an extracellular glycoprotein belonging to the BM-40/SPARC/osteonectin family of proteins containing both extracellular calcium-binding and follistatin-like domains. FSTL-1 was originally cloned from an osteoblastic cell line as a TGF-β-inducible gene. The protein occurs in two isoforms resulting from differential sialylation. The domain structure of Fstl-1 has been determined by sequence comparisons. The protein has a Follistatin N-terminal domain-like structure at amino acids 29-52 (note: amino acid residue numbers are for the bovine polypeptide (GenBank Accession No. NP_{—}001017950)—alignment with the human polypeptide (GenBank Accession No. AAH00055) permits delineation of corresponding domains in the human polypeptide), a Kazal serine protease inhibitor domain at amino acids 52-97, a low complexity region between amino acids 102-111, a region with insignificant similarity to other domains at amino acids 112-146, two EF Hand calcium-binding like domains at residues 147-174 and 196-224, and a coiled coil domain at residues 269-302. Useful portions of the polypeptide can include, for example, one or more of these structural domains, e.g., a Follistatin-like N terminal domain, a Kazal serine protease inhibitor-like domain, or an EF Hand domain.

The term “tissue” refers to a group or layer of similarly specialized cells, which together perform certain special functions. The term “tissue-specific” refers to a source of cells from a specific tissue. The term “tissue” is intended to include, blood, blood preparations such as plasma and serum, bones, joints, muscles, smooth muscles, and organs.

As used herein, the term “biological sample” also refers to a cell or population of cells or a quantity of tissue or fluid from a subject. Most often, the sample has been removed from a subject, but the term “biological sample” can also refer to cells or tissue analyzed in vivo, i.e. without removal from the subject. Often, a “biological sample” will contain cells from a subject, but the term can also refer to non-cellular biological material, such as non-cellular fractions of blood, saliva, or urine, that can be used to measure protein phosphorylation levels. As used herein, a “biological sample” or “tissue sample” refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, a biological sample is from a resection, bronchoscopic biopsy, or core needle biopsy of a primary, secondary or metastatic tumor, or a cellblock from pleural fluid. In addition, fine needle aspirate biological samples are also useful. In some embodiments, a biological sample is primary ascite cells. Samples can be fresh, frozen, fixed or optionally paraffin-embedded, frozen or subjected to other tissue preservation methods, including for example methods to preserve the phosphorylation status of polypeptides in the biological sample. A biological sample can also mean a sample of biological tissue or fluid that comprises protein or cells. Such samples include, but are not limited to, tissue isolated from subjects or animals. Biological samples may also include sections of tissues such as biopsy and autopsy samples, frozen sections taken for histological purposes, blood, plasma, serum, sputum, stool, tears, mucus, hair, and skin. Biological samples also include explants and primary and/or transformed cell cultures derived from patient tissues. A biological sample may be provided by removing a sample of cells from subject, but can also be accomplished by using previously isolated cells (e.g., isolated by another person, at another time, and/or for another purpose), or by performing the methods of the invention in vivo. Archival tissues, such as those having treatment or outcome history may also be used. Biological samples include, but are not limited to, tissue biopsies, scrapes (e.g. buccal scrapes), whole blood, plasma, serum, urine, saliva, cell culture, or cerebrospinal fluid. Biological samples also include tissue biopsies, cell culture. The biological sample can be obtained by removing a sample of cells from a subject, but can also be accomplished by using previously isolated cells (e.g. isolated by another person), or by performing the methods of the invention in vivo. Such samples include, but are not limited to, whole blood, cultured cells, primary cell preparations, sputum, amniotic fluid, tissue or fine needle biopsy samples, peritoneal fluid, and pleural fluid, among others. In some embodiments a biological sample is taken from a human patient, and in alternative embodiments the biological sample is taken from any mammal, such as rodents, animal models of diseases, commercial animals, companion animals, dogs, cats, sheep, cattle, and pigs, etc. The biological sample can be pretreated as necessary for storage or preservation, by dilution in an appropriate buffer solution or concentrated, if desired. Any of a number of standard aqueous buffer solutions, employing one of a variety of buffers, such as phosphate, Tris, or the like, at physiological pH can be used. The biological sample can in certain circumstances be stored for use prior to use in the assay as disclosed herein. Such storage can be at +4 C or frozen, for example at −20 C or −80 C, provided suitable cryopreservation agents are used to maintain cell viability once the cells are thawed.

The terms “homology”, “identity” and “similarity” refer to the degree of sequence similarity between two polypeptides or between two optimally aligned nucleic acid molecules. Homology and identity can each be determined by comparing a position in each sequence which can be aligned for purposes of comparison. For example, it is based upon using a standard homology software in the default position, such as BLAST, version 2.2.14. When an equivalent position in the compared sequences is occupied by the same base or amino acid, then the molecules are identical at that position; when the equivalent site occupied by similar amino acid residues (e.g., similar in steric and/or electronic nature such as, for example conservative amino acid substitutions), then the molecules can be referred to as homologous (similar) at that position. Expression as a percentage of homology/similarity or identity refers to a function of the number of similar or identical amino acids at positions shared by the compared sequences, respectfully. A sequence which is “unrelated” shares less than 40% identity, though preferably less than 25% identity with the sequences as disclosed herein.

As used herein, the terms “homologous” or “homologues” are used interchangeably, and when used to describe a polynucleotide or polypeptide, indicate that two polynucleotides or polypeptides, or designated sequences thereof, when optimally aligned and compared, for example using BLAST, version 2.2.14 with default parameters for an alignment (see herein) are identical, with appropriate nucleotide insertions or deletions or amino-acid insertions or deletions, in at least 70% of the nucleotides, usually from about 75% to 99%, and more preferably at least about 98 to 99% of the nucleotides. The term “homolog” or “homologous” as used herein also refers to homology with respect to structure and/or function. That is, a polypeptide that performs the same function as a given polypeptide in another species can be viewed as a homolog of that given polypeptide. Determination of homologs of genes or polypeptides can be easily ascertained by the skilled artisan.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Where necessary or desired, optimal alignment of sequences for comparison can be conducted, for example, by the local homology algorithm of Smith and Waterman (Adv. Appl. Math. 2:482 (1981), which is incorporated by reference herein), by the homology alignment algorithm of Needleman and Wunsch (J. Mol. Biol. 48:443-53 (1970), which is incorporated by reference herein), by the search for similarity method of Pearson and Lipman (Proc. Natl. Acad. Sci. USA 85:2444-48 (1988), which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al. (eds.), Current Protocols in Molecular Biology, 4th ed., John Wiley and Sons, New York (1999)).

One example of a useful algorithm is PILEUP. PILEUP creates a multiple sequence alignment from a group of related sequences using progressive, pairwise alignments to show the percent sequence identity. It also plots a tree or dendogram showing the clustering relationships used to create the alignment. PILEUP uses a simplification of the progressive alignment method of Feng and Doolittle (J. Mol. Evol. 25:351-60 (1987), which is incorporated by reference herein). The method used is similar to the method described by Higgins and Sharp (Comput. Appl. Biosci. 5:151-53 (1989), which is incorporated by reference herein). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, which is described by Altschul et al. (J. Mol. Biol. 215:403-410 (1990), which is incorporated by reference herein). (See also Zhang et al., Nucleic Acid Res. 26:3986-90 (1998); Altschul et al., Nucleic Acid Res. 25:3389-402 (1997), which are incorporated by reference herein). Software for performing BLAST analyses is publicly available through the National Center for Biotechnology Information internet web site. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff and Henikoff, Proc. Natl. Acad. Sci. USA 89:10915-9 (1992), which is incorporated by reference herein) alignments (B) of 50, expectation (E) of 10, M=5, N=-4, and a comparison of both strands.

In addition to calculating percent sequence identity, the BLAST algorithm also performs a statistical analysis of the similarity between two sequences (see, e.g., Karlin and Altschul, Proc. Natl. Acad. Sci. USA 90:5873-77 (1993), which is incorporated by reference herein). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference amino acid sequence if the smallest sum probability in a comparison of the test amino acid to the reference amino acid is less than about 0.1, more typically less than about 0.01, and most typically less than about 0.001.

As used herein, the term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T. C, G, U or I) or amino acid residue occurs in both sequences to yield the number of matched positions, dividing the number of matched positions by the total number of positions in the comparison window (i.e., the window size), and multiplying the result by 100 to yield the percentage of sequence identity.

The term “variant” as used herein refers to a polypeptide or nucleic acid that differs from the naturally occurring polypeptide or nucleic acid by one or more amino acid or nucleic acid deletions, additions, substitutions or side-chain modifications, yet retains one or more specific functions or biological activities of the naturally occurring molecule. Amino acid substitutions include alterations in which an amino acid is replaced with a different naturally-occurring or a non-conventional amino acid residue. Such substitutions may be classified as “conservative,” in which case an amino acid residue contained in a polypeptide is replaced with another naturally occurring amino acid of similar character either in relation to polarity, side chain functionality or size. Substitutions encompassed by variants as described herein may also be “non conservative,” in which an amino acid residue which is present in a peptide is substituted with an amino acid having different properties (e.g., substituting a charged or hydrophobic amino acid with alanine), or alternatively, in which a naturally-occurring amino acid is substituted with a non-conventional amino acid. Also encompassed within the term “variant,” when used with reference to a polynucleotide or polypeptide, are variations in primary, secondary, or tertiary structure, as compared to a reference polynucleotide or polypeptide, respectively (e.g., as compared to a wild-type polynucleotide or polypeptide). A “variant” of an Fstl1 polypeptide refers to a molecule substantially similar in structure and function to that of a polypeptide of SEQ ID NO: 1, where the function is the ability to mediate, effect or facilitate transport of an associated or fused polypeptide across a cell membrane of a living cell from a subject. In some embodiments, a variant of SEQ ID NO: 1 is a fragment of SEQ ID NO: 1 as disclosed herein.

A molecule is said to be “substantially similar” to another molecule if both molecules have substantially similar structures (i.e., they are at least 50% similar in amino acid sequence as determined by BLASTp alignment set at default parameters) and are substantially similar in at least one relevant function (here, for example, at least 50% as active in mediating, effecting or facilitating transport of an associated or fused polypeptide across the membrane of an intact, living cell). Transmembrane transport can be measured by methods known in the art. To avoid any confusion, a preferred method is that described by Kushner et. al., 2003, Proc. Natl. Acad. Sci. U S A. 100: 6652-6657.

The term “substantially similar,” when used in reference to a variant of Fstl1 or a functional derivative of Fstl1 as compared to the Fstl1 protein encoded by SEQ ID NO: 1 means that a particular subject sequence, for example, an Fstl1 fragment or Fstl1 variant or Fstl1 derivative sequence, varies from the sequence of the Fstl1 polypeptide encoded by SEQ ID NO: 1 by one or more substitutions, deletions, or additions relative to SEQ ID NO: 1, but retains at least 50% of the normal activity of SEQ ID NO: 1, and preferably higher, e.g., at least 60%, 70%, 80%, 90% or more exhibited by the Fstl1 protein of SEQ ID NO: 1. In determining polynucleotide sequences, all subject polynucleotide sequences capable of encoding substantially similar amino acid sequences are considered to be substantially similar to a reference polynucleotide sequence, regardless of differences in codon sequence. A nucleotide sequence is “substantially similar” to a given Fstl1 nucleic acid sequence if: (a) the nucleotide sequence is hybridizes to the coding regions of the native Fstl1 sequence (i.e. SEQ ID NO: 2), or (b) the nucleotide sequence is capable of hybridization to nucleotide sequence of Fstl1 encoded by SEQ ID NO: 2 under moderately stringent conditions and has biological activity similar to the native Fstl1 protein of SEQ ID NO: 1; or (c) the nucleotide sequences are degenerate as a result of the genetic code relative to the nucleotide sequences defined in (a) or (b). Substantially similar proteins will typically be greater than about 80% similar to the corresponding sequence of the native protein.

Variants can include conservative or non-conservative amino acid changes, as described below. Polynucleotide changes can result in amino acid substitutions, additions, deletions, fusions and truncations in the polypeptide encoded by the reference sequence. Variants can also include insertions, deletions or substitutions of amino acids, including insertions and substitutions of amino acids and other molecules) that do not normally occur in the peptide sequence that is the basis of the variant, for example but not limited to insertion of ornithine which do not normally occur in human proteins. “Conservative amino acid substitutions” result from replacing one amino acid with another having similar structural and/or chemical properties. Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W. H. Freeman and Company (1984).)

The choice of conservative amino acids may be selected based on the location of the amino acid to be substituted in the peptide, for example if the amino acid is on the exterior of the peptide and exposed to solvents, or on the interior and not exposed to solvents. Selection of such conservative amino acid substitutions is within the skill of one of ordinary skill in the art and is described, for example by Dordo et al., J. Mol Biol, 1999, 217, 721-739 and Taylor et al., J. Theor. Biol. 119(1986);205-218 and S. French and B. Robson, J. Mol. Evol. 19(1983)171. Accordingly, one can select conservative amino acid substitutions suitable for amino acids on the exterior of a protein or peptide (i.e. amino acids exposed to a solvent). These substitutions include, but are not limited to the following: substitution of Y with F, T with S or K, P with A, E with D or Q, N with D or G, R with K, G with N or A, T with S or K, D with N or E, I with L or V, F with Y, S with T or A, R with K, G with N or A, K with R, A with S, K or P.

In alternative embodiments, one can also select conservative amino acid substitutions suitable for amino acids on the interior of a protein or peptide. For example, one can use suitable conservative substitutions for amino acids in the interior of a protein or peptide (i.e. the amino acids are not exposed to a solvent). For example, one can use the following conservative substitutions: where Y is substituted with F, T with A or S, I with L or V, W with Y, M with L, N with D, G with A, T with A or S, D with N, I with L or V, F with Y or L, S with A or T and A with S, G, T or V. In some embodiments, Fstl1 polypeptides including non-conservative amino acid substitutions are also encompassed within the term “variants.” A variant of an Fstl1 polypeptide, for example a variant of SEQ ID NO: 1 is meant to refer to any molecule substantially similar in structure (i.e., having at least 50% homology as determined by BLASTp analysis using default parameters) and function (i.e., at least 50% as effective) as a polypeptide of SEQ ID NO: 1.

As used herein, the term “non-conservative” refers to substituting an amino acid residue for a different amino acid residue that has different chemical properties. Non-limiting examples of non-conservative substitutions include aspartic acid (D) being replaced with glycine (G); asparagine (N) being replaced with lysine (K); and alanine (A) being replaced with arginine (R).

The term “derivative” as used herein refers to peptides which have been chemically modified, for example by ubiquitination, labeling, pegylation (derivatization with polyethylene glycol) or addition of other molecules. A molecule is also a “derivative” of another molecule when it contains additional chemical moieties not normally a part of the molecule. Such moieties can improve the molecule's solubility, absorption, biological half life, etc. The moieties can alternatively decrease the toxicity of the molecule, or eliminate or attenuate an undesirable side effect of the molecule, etc. Moieties capable of mediating such effects are disclosed in Remington's Pharmaceutical Sciences, 18th edition, A. R. Gennaro, Ed., MackPubl., Easton, Pa. (1990).

The term “functional” when used in conjunction with “derivative” or “variant” refers to a protein molecule which possess a biological activity that is substantially similar to a biological activity of the entity or molecule of which it is a derivative or variant. By “substantially similar” in this context is meant that the biological activity, e.g., transmembrane transport of associated polypeptides is at least 50% as active as a reference, e.g., a corresponding wild-type polypeptide, and preferably at least 60% as active, 70% as active, 80% as active, 90% as active, 95% as active, 100% as active or even higher (i.e., the variant or derivative has greater activity than the wild-type), e.g., 110% as active, 120% as active, or more.

“Insertions” or “deletions,” as the terms are used herein, are typically in the range of about 1 to 5 amino acids. The variation permitted in view of maintaining function can be experimentally determined by producing the peptide synthetically while systematically making insertions, deletions, or substitutions of nucleotides in the sequence using recombinant DNA techniques.

The term “specifically binds” refers to binding with a K_{d} of 10 micromolar or less, preferably 1 micromolar or less, more preferably 100 nM or less, 10 nM or less, or 1 nM or less.

By “substantially pure” is meant a nucleic acid, polypeptide, or other molecule that has been separated from the components that naturally accompany it. Typically, a polypeptide is substantially pure when it is at least about 60%, or at least about 70%, at least about 80%, at least about 90%, at least about 95%, or even at least about 99%, by weight, free from the proteins and naturally occurring organic molecules with which it is naturally associated. A substantially pure polypeptide may be obtained, for example, by extraction from a natural source, by expression of a recombinant nucleic acid in a cell that does not normally express that protein, or by chemical synthesis.

As used herein the term “reference level” refers to a Fstl1 expression level in a particular biological sample which provides a baseline against which to measure a Fstl1 expression level from the test biological sample. As an illustrative example, the reference level for Fstl1 expression can be calculated as the average level of the Fstl1 gene and/or protein expression from a plurality of biological samples obtained from a plurality of subjects with similar demographics (i.e. age, gender, weight, ethnicity and the like). As another illustrative example only, a reference level for Fstl1 expression can be from a plurality of subjects that do not have risk of, or have cardiac stress and/or skeletal muscle stress. As another illustrative example only, a reference level for Fstl1 expression can be from the same subject taken at an earlier timepoint. Typically, a reference level is normalized to “0” value, and an increase, for example at least about a 30% increase in the Fstl1 expression level measured by the determination module or in the system and methods as disclosed herein relative to the reference level would indicate a subject would have an risk of cardiac stress and/or skeletal muscle stress (i.e. a positive cardiac stress and/or skeletal muscle stress test result). In some embodiments, a reference level is normalized to “0” value, and a decrease, for example at least about a 30% decrease in the Fstl1 expression level measured by the determination module or in the system and methods as disclosed herein relative to the reference level would indicate a subject would have an risk of diabetes and/or metabolic dysfunction (i.e. a positive diabetes and/or metabolic dysfunction test result). A reference Fstl1 expression level can be from an individual not affected by a given pathology (i.e not affected with cardiac stress and/or skeletal muscle stress and/or diabetes and/or metabolic dysfunction), or, alternatively, from the same individual being tested, where the biological sample for the reference Fstl1 expression was taken at an at least one earlier time point (i.e. to, t₁, t₂ etc). A reference can also be a pooled sample, taken from a plurality of individuals not affected by the pathology in question (i.e not affected with cardiac stress and/or skeletal muscle stress and/or diabetes and/or metabolic dysfunction). Where appropriate, a reference can also be a fixed reference level of a Fstl1 expression level, where a test Fstl1 expression level above the fixed reference level (i.e. at least about 30% above the fixed reference level) a subject is identified as having a risk of cardiac stress and/or skeletal muscle stress (i.e. a positive cardiac stress and/or skeletal muscle stress test result), or alternatively, where a test Fstl1 expression level below the fixed reference level (i.e. at least about 30% below the fixed reference level) a subject is identified as having a risk of diabetes and/or metabolic dysfunction (i.e. a positive diabetes and/or metabolic dysfunction test result). It is preferred that a reference sample be from an individual or group of individuals of similar characteristics to the tested individual, e.g., that the reference be taken from individuals of similar age, gender, rave or ethnic background, etc. In some embodiments, other reference levels can also be used, for example a positive reference Fstl1 expression level can be used as a positive control for a subject having a risk of cardiac stress and/or skeletal muscle stress, and a different positive reference Fstl1 expression level can be used as a positive control for a subject having a risk of diabetes and/or metabolic dysfunction. Typically, where a positive reference level is used, if the Fstl1 expression level in the test biological sample as disclosed herein is substantially the same or close in the value of the positive reference Fstl1 expression level, it would indicate a positive test result for cardiac stress and/or skeletal muscle stress and/or diabetes and/or metabolic dysfunction, respectively.

The term “reduced” or “reduce” or “decrease” as used herein generally means a decrease by a statistically significant amount relative to a reference. However, for avoidance of doubt, “reduced” means statistically significant decrease of at least 10% as compared to a reference level, for example a decrease by at least 20%, at least 30%, at least 40%, at least t 50%, or least 60%, or least 70%, or least 80%, at least 90% or more, up to and including a 100% decrease (i.e. absent level as compared to a reference sample), or any decrease between 10-100% as compared to a reference level, as that term is defined herein.

The term “low” as used herein generally means lower by a statically significant amount; for the avoidance of doubt, “low” means a statistically significant value at least 10% lower than a reference level, for example a value at least 20% lower than a reference level, at least 30% lower than a reference level, at least 40% lower than a reference level, at least 50% lower than a reference level, at least 60% lower than a reference level, at least 70% lower than a reference level, at least 80% lower than a reference level, at least 90% lower than a reference level, up to and including 100% lower than a reference level (i.e. absent level as compared to a reference sample).

The terms “increased” or “increase” as used herein generally mean an increase by a statically significant amount; for the avoidance of doubt, “increased” means a statistically significant increase of at least 10% as compared to a reference level, including an increase of at least 20%, at least 30%, at least 40%, at least 50%, at least 60%, at least 70%, at least 80%, at least 90%, at least 100% or more, including, for example at least 2-fold, at least 3-fold, at least 4-fold, at least 5-fold, at least 10-fold increase or greater as compared to a reference level, as that term is defined herein.

The term “high” as used herein generally means a higher by a statically significant amount relative to a reference; for the avoidance of doubt, “high” means a statistically significant value at least 10% higher than a reference level, for example at least 20% higher, at least 30% higher, at least 40% higher, at least 50% higher, at least 60% higher, at least 70% higher, at least 80% higher, at least 90% higher, at least 100% higher, at least 2-fold higher, at least 3-fold higher, at least 4-fold higher, at least 5-fold higher, at least 10-fold higher or more, as compared to a reference level.

As used herein, the terms “treat,” “treating,” and “treatment” refer to the alleviation or measurable lessening of one or more symptoms or measurable markers of a disease or disorder; while not intending to be limited to such, disease or disorders of particular interest include ischemic or ischemia/reperfusion injury and diabetes. Measurable lessening includes any statistically significant decline in a measurable marker or symptom.

As used herein, the terms “prevent,” “preventing” and “prevention” refer to the avoidance or delay in manifestation of one or more symptoms or measurable markers of a disease or disorder. A delay in the manifestation of a symptom or marker is a delay relative to the time at which such symptom or marker manifests in a control or untreated subject with a similar likelihood or susceptibility of developing the disease or disorder. The terms “prevent,” “preventing” and “prevention” include not only the complete avoidance or prevention of symptoms or markers, but also a reduced severity or degree of any one of those symptoms or markers, relative to those symptoms or markers arising in a control or non-treated individual with a similar likelihood or susceptibility of developing the disease or disorder, or relative to symptoms or markers likely to arise based on historical or statistical measures of populations affected by the disease or disorder. By “reduced severity” is meant at least a 10% reduction in the severity or degree of a symptom or measurable disease marker, relative to a control or reference, e.g., at least 15%, 20%, 30%, 40%, 50%, 60%, 70%, 80%, 90%, 95%, 99% or even 100% (i.e., no symptoms or measurable markers).

As used herein, the term “ischemic injury” refers to conditions directly associated with reduced blood flow to tissue, for example due to a clot or obstruction of blood vessels which supply blood to the subject tissue and which result, inter alia, in lowered oxygen transport to such tissue, impaired tissue performance, tissue dysfunction and/or necrosis and can contribute to the pathogenesis of heart failure. Alternatively, where blood flow or organ perfusion may be quantitatively adequate, the oxygen carrying capacity of the blood or organ perfusion medium may be reduced, e.g., in hypoxic environment, such that oxygen supply to the tissue is lowered, and impaired tissue performance, tissue dysfunction, and/or tissue necrosis ensues. “Ischemia/reperfusion injury” refers to a subset of ischemic injury in which injury involves a period of reduced blood flow, followed by at least partial restoration of the blood flow. Ischemia/reperfusion injury involves an inflammatory response and oxidative damage accompanied by apoptosis that occur when blood flow has been restored to a tissue subjected to an interruption in blood flow. As used herein, the term “ischemic limb disease” refers to any disease resulting from lack of blood flow to a superficial limb or extremity (e.g., an arm, leg, hand, foot, toe, finger etc.). Ischemic limb disease results from complications due to diabetes or atherosclerosis, among others.

The term “in vivo” refers to assays or processes that occur in an animal.

The term “ex vivo” refers to assays that are performed using a living cell with an intact membrane that is outside of the body, e.g., explants, cultured cells, including primary cells and cell lines, transformed cell lines, and extracted tissue or cells, including blood cells, among others.

The term “mammal” is intended to encompass a singular “mammal” and plural “mammals,” and includes, but is not limited to humans; primates such as apes, monkeys, orangutans, and chimpanzees; canids such as dogs and wolves; felids such as cats, lions, and tigers; equids such as horses, donkeys, and zebras, food animals such as cows, pigs, and sheep; ungulates such as deer and giraffes; rodents such as mice, rats, hamsters and guinea pigs; and bears. In some embodiments, a mammal is a human.

The terms “patient”, “subject” and “individual” are used interchangeably herein, and refers to any animal in which it is useful to diagnose or determine cardiac stress or injury, and/or skeletal muscle stress, for example to diagnose if the subject has a disease or condition, or is likely to develop a disease or condition, and a subject to whom treatment including prophylaxis treatment can be provided and is desirable. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. The subject can be a mammal, for example a human, or can be a wild, domestic, commercial or companion animal. While in one embodiment of the invention it is contemplated that the cardiac stress or injury, and/or skeletal muscle stress test suitable for the diagnostic use in humans, it is also applicable to all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is a wild animal, for example a bird such as for the diagnosis of avian flu. In some embodiments, the subject is an experimental animal or animal substitute as a disease model. The subject may be a subject in need of veterinary treatment, including treatment of companion animals such as dogs and cats, and domestic animals such as horses, ponies, donkeys, mules, llama, alpaca, pigs, cattle and sheep, or zoo animals such as primates, felids, canids, bovids, and ungulates, or livestock animals such as pigs, cattle and sheep.

The term “computer” can refer to any non-human apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

The term “computer-readable medium” may refer to any storage device used for storing data accessible by a computer, as well as any other means for providing access to data by a computer. Examples of a storage-device-type computer-readable medium include: a magnetic hard disk; a floppy disk; an optical disk, such as a CD-ROM and a DVD; a magnetic tape; a memory chip.

The term “software” can refer to prescribed rules to operate a computer. Examples of software include: software; code segments; instructions; computer programs; and programmed logic.

The term a “computer system” may refer to a system having a computer, where the computer comprises a computer-readable medium embodying software to operate the computer.

The term “proteomics” may refer to the study of the expression, structure, and function of proteins within cells, including the way they work and interact with each other, providing different information than genomic analysis of gene expression.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

Other than in the operating examples, or where otherwise indicated, all numbers expressing quantities of ingredients or reaction conditions used herein should be understood as modified in all instances by the term “about.” The term “about” when used in connection with percentages can mean ±1%. The present invention is further explained in detail by the following, including the Examples, but the scope of the invention should not be limited thereto.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Other features and advantages of the invention will be apparent from the following Detailed Description, the drawings, and the claims.

### Systems and Computer Readable Media

In one aspect, provided herein is a system for measuring a Fstl1 expression level in a biological sample obtained from a subject, the system comprising a computer processor and a computer-readable physical storage medium having instructions recorded thereon sufficient to implement a process, employing the computer processor, for measuring a cell-mediated immune response, the instructions for said process comprising: a) instructions for receiving data regarding the level of Fstl1 expression (i.e. Fstl1 polypeptide expression or Fstl1 gene expression, i.e. Fstl1 mRNA expression) in a biological sample; and b) instructions for comparing the level of said Fstl1 expression level in said biological sample with at least one reference level of Fstl1 expression, c) instructions for transmitting to a user interface a result of said comparison, wherein an increase in the level of said Fstl1 expression level in said biological sample from the subject as compared to a reference Fstl1 expression level indicates a subject is likely to have a cardiac stress and/or skeletal muscle stress.

In another aspect, provided herein is a computer-readable physical storage medium having instructions recorded thereon sufficient to implement a process, employing a computer processor, for measuring the Fstl1 expression level in a biological sample, the instructions for said process comprising: a) instructions for receiving data regarding the Fstl1 1 expression level (i.e. Fstl1 polypeptide expression or Fstl1 gene expression, i.e. Fstl1 mRNA expression) in a biological sample, b) instructions for comparing the level of said Fstl1 1 expression level (i.e. Fstl1 polypeptide expression or Fstl1 gene expression, i.e. Fstl1 mRNA expression) in said biological sample with a reference level of said Fstl1 1 expression level, c) instructions for transmitting to a user interface a result of said comparison, wherein an increase in the level of said Fstl1 expression level in said biological sample from the subject as compared to a reference Fstl1 expression level indicates a subject is likely to have a cardiac stress and/or skeletal muscle stress.

In another aspect, provided herein is a system for detecting cardiac stress and/or skeletal muscle stress in a subject, the system comprising a computer processor a computer-readable physical storage medium having instructions recorded thereon sufficient to implement a process, employing the computer processor, for measuring a cell-mediated immune response, the instructions for said process comprising:

a) instructions for receiving data regarding the Fstl1 1 expression level (i.e. Fstl1 polypeptide expression or Fstl1 gene expression, i.e. Fstl1 mRNA expression) in a biological sample; and

b) instructions for comparing the level of said Fstl1 1 expression level (i.e. Fstl1 polypeptide expression or Fstl1 gene expression, i.e. Fstl1 mRNA expression) in said biological sample with a reference Fstl1 1 expression level; and

c) instructions for transmitting to a user interface a result of the comparison of (b), wherein an increase in the level of said Fstl1 expression level in said biological sample from the subject as compared to a reference Fstl1 expression level indicates a subject is likely to have a cardiac stress and/or skeletal muscle stress.

In another aspect, provided herein is a computer-readable physical storage medium having instructions recorded thereon sufficient to implement a process, employing a computer processor, for diagnosing cardiac stress and/or skeletal muscle stress in a subject, the instructions for said process comprising:

a) instructions for receiving data regarding Fstl1 1 expression level (i.e. Fstl1 polypeptide expression or Fstl1 gene expression, i.e. Fstl1 mRNA expression); and

b) instructions for comparing the level of said Fstl1 1 expression level (i.e. Fstl1 polypeptide expression or Fstl1 gene expression, i.e. Fstl1 mRNA expression) in said biological sample with a reference Fstl1 1 expression level; and

c) instructions for transmitting to a user interface a result of the comparison of (b), wherein an increase in the level of said Fstl1 expression level in said biological sample from the subject as compared to a reference Fstl1 expression level indicates a subject is likely to have a cardiac stress and/or skeletal muscle stress.

In some embodiments, the instructions in step (c) are instructions for transmitting to a user interface a result of the comparison of (b), wherein an increase in the level of said Fstl1 expression level in said biological sample from the subject above a threshold as compared to a reference Fstl1 expression level indicates a subject is likely to have a cardiac stress and/or a skeletal muscle stress. In some embodiments, the instructions in step (c) are instructions for transmitting to a user interface a result of the comparison of (b), wherein an increase in the level of said Fstl1 expression level in said biological sample from the subject of at least about 30% above as compared to a reference Fstl1 expression level indicates a subject is likely to have a cardiac stress and/or a skeletal muscle stress

In some embodiments, the instructions in step (c) are instructions for transmitting to a user interface a result of the comparison of (b), wherein a decrease in the level of said Fstl1 expression level in said biological sample from the subject as compared to a reference Fstl1 expression level indicates a subject is likely to have a diabetes an/or a metabolic dysfunction.

Computer-readable physical storage media useful in various embodiments include any physical computer-readable storage medium, e.g., magnetic and optical computer-readable storage media, among others. Carrier waves and other signal-based storage or transmission media are not included within the scope of physical computer-readable storage media encompassed by the term and useful according to the invention.

A user interface useful in various embodiments includes, for example, a display screen or a printer or other means for providing a readout of the result of a computer-mediated process. A user interface can also include, for example, an address in a network or on the world wide web to which the results of a process are transmitted and made accessible to one or more users. For example, the user interface can include a graphical user interface comprising an access element that permits entry of data regarding Fstl1 release in a biological sample, as well as an access element that provides a graphical read out of the results of a comparison transmitted to or made available by a processor following execution of the instructions encoded on a computer-readable medium.

Embodiments of the invention also provide for systems (and computer readable medium for causing computer systems) to perform a method for determining whether at biological test sample obtained from a subject has a positive CMI response to a target antigen, or alternatively has increased risk of having a specific pathology based on Fstl1 expression level information.

Embodiments of the invention have been described through functional modules, which are defined by computer executable instructions recorded on computer readable media and which cause a computer to perform method steps when executed. The modules have been segregated by function for the sake of clarity. However, it should be understood that the modules need not correspond to discreet blocks of code and the described functions can be carried out by the execution of various code portions stored on various media and executed at various times. Furthermore, it should be appreciated that the modules may perform other functions, thus the modules are not limited to having any particular functions or set of functions.

The computer readable media can be any available tangible media that can be accessed by a computer. Computer readable media includes volatile and nonvolatile, removable and non-removable tangible media implemented in any method or technology for storage of information such as computer readable instructions, data structures, program modules or other data. Computer readable media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media, or computer readable medium 200, may define instructions, for example, as part of one or more programs, that, as a result of being executed by a computer, instruct the computer to perform one or more of the functions described herein (e.g., in relation to system 10, or computer readable medium 200), and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of system 10, or computer readable medium 200 described herein, may be distributed across one or more of such components, and may be in transition there between.

The computer-readable media may be transportable such that the instructions stored thereon can be loaded onto any computer resource to implement the aspects of the present invention discussed herein. In addition, it should be appreciated that the instructions stored on the computer readable media, or the computer-readable medium 200, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The functional modules of certain embodiments of the invention include a determination module, a storage device, a comparison module and a display module. The functional modules can be executed on one, or multiple, computers, or by using one, or multiple, computer networks. The determination module 40 has computer executable instructions to provide sequence information in computer readable form. As used herein, “Fstl1 expression level information” refers to the expression level, preferably protein expression level of the Fstl1 polypeptide of SEQ ID NO: 1 or the gene expression level of the Fstl1 mRNA of SEQ ID NO: 2 and fragments and variants thereof. Moreover, information “related to” the Fstl1 expression level information includes detection of the presence or absence of precursor proteins of the Fstl1 polypeptide, or the presence or absence of messenger RNA sequence encoding Fstl1 polypeptide), determination of the concentration and level of protein expression of Fstl1 in the biological sample (e.g., amino acid sequence expression levels, or in some embodiments nucleotide (RNA or DNA) expression levels), and the like. The term “Fstl1 expression level information” is intended to include the presence or absence of post-translational modifications (e.g. phosphorylation, glycosylation, summylation, famesylation, and the like) of the measured Fstl1 polypeptide (i.e. secreted Fstl1 polypeptide), the presence or absence of the measured Fstl1 polypeptide and the level of protein expression of the Fstl1 polypeptide.

As an example, determination modules 40 for determining Fstl1 expression level information may include known systems for automated protein expression level determination, including for example, but not limited to, mass spectrometry systems including Matrix Assisted Laser Desorption Ionization—Time of Flight (MALDI-TOF) systems and SELDI-TOF-MS ProteinChip array profiling systems; systems for analyzing gene expression data (see, for example, published U.S. Patent Application, Pub. No. U.S. 2003/0194711, which is incorporated herein in its entirety by reference); systems for array based expression analysis: e.g., HT array systems and cartridge array systems such as GENECHIP® AUTOLOADER, COMPLETE GENECHIP® Instrument System, GENECHIP® Fluidics Station 450, GENECHIP® Hybridization Oven 645, GENECHIP® QC Toolbox Software Kit, GENECHIP® Scanner 3000 7G plus Targeted Genotyping System, GENECHIP® Scanner 3000 7G Whole-Genome Association System, GENETITAN™ Instrument, and GENECHIP® Array Station (each available from Affymetrix, Santa Clara, Calif.); automated ELISA systems (e.g., DSX® or DS2® (available from Dynax, Chantilly, Va.) or the TRITURUS® (available from Grifols USA, Los Angeles, Calif.), The MAGO® Plus (available from Diamedix Corporation, Miami, Fla.) ; Densitometers (e.g. X-Rite-508-SPECTRO DENSITOMETER® (available from RP IMAGING™, Tucson, Ariz.), The HYRYS™ 2 HIT densitometer (available from Sebia Electrophoresis, Norcross, Ga.); automated Fluorescence insitu hybridization systems (see for example, U.S. Pat. No. 6,136,540); 2D gel imaging systems coupled with 2-D imaging software; microplate readers; Fluorescence activated cell sorters (FACS) (e.g. Flow Cytometer FACSVantage SE, (available from Becton Dickinson, Franklin Lakes, N.J.); and radio isotope analyzers (e.g. scintillation counters).

In some embodiments, the determination module 40 has computer executable instructions to provide sequence information in computer readable form. As an example, a determination mode 40 for determining the level of Fstl1 polypeptide by binding of a protein-binding molecule to a protein, for example but not limited to the binding of an anti-Fstl1 antibody to a Fstl1 polypeptide include for example but are not limited to automated immunohistochemistry apparatus, for example, robotically automated immunohistochemistry apparatus which in an automated system section the tissue or biological sample specimen, prepare slides, perform immunhistochemistry procedure and detect intensity of immunostaining, such as intensity of anti-Fstl1 antibody staining in the biological sample or tissue and produce output data. Examples of such automated immunohistochemistry apparatus are commercially available, for example such Autostainers 360, 480, 720 and Labvision PT module machines from LabVision Corporation, which are disclosed in U.S. Pat. Nos. 7,435,383; 6,998,270; 6,746,851, 6,735,531; 6,349,264; and 5,839; 091 which are incorporated herein in their entirety by reference. Other commercially available automated immunohistochemistry instruments are also encompassed for use in the present invention, for example, but not are limited BOND™ Automated Immunohistochemistry & In Situ Hybridization System, Automate slide loader from GTI vision. Automated analysis of immunohistochemistry can be performed by commercially available systems such as, for example, IHC Scorer and Path EX, which can be combined with the Applied spectral Images (ASI) CytoLab view, also available from GTI vision or Applied Spectral Imaging (ASI) which can all be integrated into data sharing systems such as, for example, Laboratory Information System (LIS), which incorporates Picture Archive Communication System (PACS), also available from Applied Spectral Imaging (ASI) (see world-wide-web: spectral-imaging.com). Other a determination module can be an automated immunohistochemistry systems such as NexES® automated immunohistochemistry (IHC) slide staining system or BenchMark® LT automated IHC instrument from Ventana Discovery SA, which can be combined with VIAS™ image analysis system also available Ventana Discovery. BioGenex Super Sensitive MultiLink® Detection Systems, in either manual or automated protocols can also be used as the detection module, preferably using the BioGenex Automated Staining Systems. Such systems can be combined with a BioGenex automated staining systems, the i6000™ (and its predecessor, the OptiMax® Plus), which is geared for the Clinical Diagnostics lab, and the GenoMx 6000™, for Drug Discovery labs. Both systems BioGenex systems perform “All-in-One, All-at-Once” functions for cell and tissue testing, such as Immunohistochemistry (IHC) and In Situ Hybridization (ISH).

As an example, a determination module 40 for determining the level of a Fstl1 polypeptide may include known systems for automated protein expression analysis including but not limited Mass Spectrometry systems including MALDI-TOF, or Matrix Assisted Laser Desorption Ionization—Time of Flight systems; SELDI-TOF-MS ProteinChip array profiling systems, e.g. Machines with Ciphergen Protein Biology System II™ software; systems for analyzing gene expression data (see for example U.S. 2003/0194711); systems for array based expression analysis, for example HT array systems and cartridge array systems available from Affymetrix (Santa Clara, Calif. 95051) AutoLoader, Complete GeneChip® Instrument System, Fluidics Station 450, Hybridization Oven 645, QC Toolbox Software Kit, Scanner 3000 7G, Scanner 3000 7G plus Targeted Genotyping System, Scanner 3000 7G Whole-Genome Association System, GeneTitan™ Instrument, GeneChip® Array Station, HT Array; an automated ELISA system (e.g. DSX® or DS2® form Dynax, Chantilly, Va. or the ENEASYSTEM III, Triturus®, The Mago® Plus); Densitometers (e.g. X-Rite-508-Spectro Densitometer®, The HYRYS™ 2 densitometer); automated Fluorescence insitu hybridization systems (see for example, U.S. Pat. No. 6,136,540); 2D gel imaging systems coupled with 2-D imaging software; microplate readers; Fluorescence activated cell sorters (FACS) (e.g. Flow Cytometer FACSVantage SE, Becton Dickinson); radio isotope analyzers (e.g. scintillation counters).

Algorithms for identifying protein expression levels and profiles, such as the total amount of Fstl1 polypeptide available in a biological sample can include the use of optimization algorithms such as the mean variance algorithm, e.g. J MP Genomics algorithm available from JMP Software.

In some embodiments of this aspect and all other aspects of the present invention a variety of software programs and formats can be used to store the Fstl1 expression level information on the storage device. Any number of data processor structuring formats (e.g., text file or database) can be employed to obtain or create a medium having recorded thereon the sequence information or expression level information.

Alternatively, determination modules 40 for determining Fstl1 expression level information may include known systems for automated detection of sequencing the nucleotide (i.e. RNA expression) of the cytokines, including sequence analysis including but not limited to Hitachi FMBIO® and Hitachi FMBIO® II Fluorescent Scanners (available from Hitachi Genetic Systems, Alameda, Calif.); Spectrumedix® SCE 9610 Fully Automated 96-Capillary Electrophoresis Genetic Analysis Systems (available from SpectruMedix LLC, State College, Pa.); ABI PRISM® 377 DNA Sequencer, ABI® 373 DNA Sequencer, ABI PRISM® 310 Genetic Analyzer, ABI PRISM® 3100 Genetic Analyzer, and ABI PRISM® 3700 DNA Analyzer (available from Applied Biosystems, Foster City, Calif.); Molecular Dynamics FluorImager™ 575, SI Fluorescent Scanners, and Molecular Dynamics FluorImager™ 595 Fluorescent Scanners (available from Amersham Biosciences UK Limited, Little Chalfont, Buckinghamshire, England); GenomyxSC™ DNA Sequencing System (available from Genomyx Corporation (Foster City, Calif.); and Pharmacia ALF™ DNA Sequencer and Pharmacia ALFexpress™ (available from Amersham Biosciences UK Limited, Little Chalfont, Buckinghamshire, England).

The Fstl1 expression level information determined in the determination module can be read by the storage device. As used herein the “storage device” 30 is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage devices 30 also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media.

Storage devices 40 are also commonly referred to in the art as “computer-readable physical storage media” which is useful in various embodiments, and can include any physical computer-readable storage medium, e.g., magnetic and optical computer-readable storage media, among others. Carrier waves and other signal-based storage or transmission media are not included within the scope of storage devices 40 or physical computer-readable storage media encompassed by the term and useful according to the invention. The storage device is adapted or configured for having recorded thereon cytokine level information. Such information may be provided in digital form that can be transmitted and read electronically, e.g., via the Internet, on diskette, via USB (universal serial bus) or via any other suitable mode of communication.

As used herein, “expression level information” refers to any nucleotide and/or amino acid expression level information of at least one Fstl1 isoform, including but not limited to full-length nucleotide sequence such as SEQ ID NO: 2 and/or amino acid sequence of Fstl1 such as SEQ ID NO: 1, as well as partial nucleotide and/or amino acid sequences, or mutated sequences. Moreover, information “related to” the Fstl1 expression level information includes detection of the presence or absence of a sequence (e.g., presence or absence of an amino acid sequence, nucleotide sequence, or post translational modification), determination of the concentration of a sequence in the sample (e.g., amino acid sequence levels, or nucleotide (RNA or DNA) expression levels, or level of post translational modification), and the like.

As used herein, “stored” refers to a process for encoding information on the storage device 30. Those skilled in the art can readily adopt any of the presently known methods for recording information on known media to generate manufactures comprising the Fstl1 expression level information.

A variety of software programs and formats can be used to store the Fstl1 expression level information on the storage device. Any number of data processor structuring formats (e.g., text file or database) can be employed to obtain or create a medium having recorded thereon the Fstl1 expression level information.

By providing Fstl1 expression level information in computer-readable form, one can use the Fstl1 expression level information on in readable form in the comparison module 80 to compare a specific Fstl1 expression profile with the reference data within the storage device 30. For example, search programs can be used to identify which isoforms of Fstl1 are expressed (reference data, e.g., presence or absence of isoform information obtained from at lease one control sample) or direct comparison of the determined Fstl1 expression level measured by the determination module can be compared to the reference data or reference Fstl1 expression level (e.g., Fstl1 level information obtained from a control sample). The comparison is made with an electronic computer in computer-readable form, and provides a computer readable comparison result which can be processed by a variety of means. Content 140 based on the comparison result can be retrieved from the comparison module 80 to indicate, for example, at least one of the following; (i) the presence or absence of cardiac stress and/or skeletal muscle stress, (ii) the severity of cardiac stress and/or skeletal muscle stress, or in some embodiments a signal to indicate the presence of, or likelihood of developing cardiac stress and/or skeletal muscle stress. In some embodiments, the comparison module indicates, for example, at least one of the following; (i) the presence or absence of diabetes, (ii) presence or absence of a metabolic dysfunction, (iii) the severity of diabetes and/or metabolic dysfunction, or (iv) the likelihood of developing diabetes and/or metabolic dysfunction.

In one embodiment the reference data stored in the storage device 30 to be read by the comparison module 80 is Fstl1 expression level information data obtained from a control biological sample of the same type as the biological sample to be tested. Alternatively, the reference data are a database, e.g., an expression level profile (RNA, protein or peptide) of Fstl1 in normal pathology unaffected subjects. In one embodiment the reference data are Fstl1 expression level information that are indicative of a not having a cardiac stress or skeletal muscle stress. . In one embodiment the reference data are Fstl1 expression level information obtained from a biological sample of the same type from the same subject at an earlier or prior timepoint. In some embodiments, the reference data is a Fstl1 expression level of the same type of biological sample from a plurality of subjects of the same or a similar demographic background (i.e. age, weight, ethnicity, gender, socioeconomic situation and the like).

In one embodiment, the reference data are electronically or digitally recorded and annotated from databases including, but not limited to protein expression databases commonly known in the art, such as Yale Protein Expression Database (YPED), as well as GenBank (NCBI) protein and DNA databases such as genome, ESTs, SNPS, Traces, Celara, Ventor Reads, Watson reads, HGTS, and the like; Swiss Institute of Bioinformatics databases, such as ENZYME, PROSITE, SWISS-2DPAGE, Swiss-Prot and TrEMBL databases; the Melanie software package or the ExPASy WWW server, and the like; the SWISS-MODEL, Swiss-Shop and other network-based computational tools; the Comprehensive Microbial Resource database (available from The Institute of Genomic Research). The resulting Fstl1 expression level information can be stored in a relational database that may be employed to determine differences in Fstl1 expression levels between the reference data or within and among genomes and different populations of individuals.

The “comparison module” 80 can use a variety of available software programs and formats for the comparison operative to compare sequence information determined in the determination module 40 to reference data. In one embodiment, the comparison module 80 is configured to use pattern recognition techniques to compare Fstl1 expression level information from one or more entries to one or more reference data patterns. The comparison module 80 may be configured using existing commercially-available or freely-available software for comparing protein expression patterns, and may be optimized for particular data comparisons that are conducted. The comparison module 80 provides computer readable information related to the Fstl1 expression level information that can include, for example, detection of the presence or absence of a Fstl1 protein or RNA, the level of Fstl1 protein expression information, detection of post-translational modification of Fstl1 protein; determination of the concentration of Fstl1 protein in the biological sample (e.g., amino acid sequence/protein expression levels, or nucleotide (RNA or DNA) expression levels, or levels of post-translational modification), or their pre-proteins or variants including homologues and post-translated modificated Fstl1 protein.

The comparison module 80, or any other module of the invention, may include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware--as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers.

In one embodiment, the comparison module 80 performs comparisons with mass-spectrometry spectra, for example comparisons of peptide fragment sequence information can be carried out using spectra processed in MATLB with script called “Qcealign” (see for example WO2007/022248, herein incorporated by reference) and “Qpeaks” (Spectrum Square Associates, Ithaca, N.Y.), or Ciphergen Peaks 2.1™ software. The processed spectra can then be aligned using alignment algorithms that align sample data to the control data using minimum entropy algorithm by taking baseline corrected data (see for example WIPO Publication WO2007/022248, herein incorporated by reference). The comparison result can be further processed by calculating ratios. Fstl1 protein expression profiles can be discerned.

In one embodiment, the comparison module 80 compares protein expression profiles. Any available comparison software can be used, including but not limited to, the Ciphergen Express (CE) and Biomarker Patterns Software (BPS) package (available from Ciphergen Biosystems, Inc., Freemont, Calif.). Comparative analysis can be done with protein chip system software (e.g., The Proteinchip Suite (available from Bio-Rad Laboratories, Hercules, Calif.). Algorithms for identifying expression profiles can include the use of optimization algorithms such as the mean variance algorithm (e.g. JMP Genomics algorithm available from JMP Software Cary, N.C.).

In one embodiment, the comparison module 80 compares gene expression profiles. For example, detection of gene expression profiles can be determined using Affymetrix Microarray Suite software version 5.0 (MAS 5.0) (available from Affymetrix, Santa Clara, Calif.) to analyze the relative abundance of a gene or genes on the basis of the intensity of the signal from probe sets, and the MAS 5.0 data files can be transferred into a database and analyzed with Microsoft Excel and GeneSpring 6.0 software (available from Agilent Technologies, Santa Clara, Calif.). The detection algorithm of MAS 5.0 software can be used to obtain a comprehensive overview of how many transcripts are detected in given samples and allows a comparative analysis of 2 or more microarray data sets.

In one embodiment of the invention, pattern comparison software is used to determine whether the Fstl1 expression level is above a threshold level (i.e. above a threshold level of at least about 30%, or at least about 40% or at least about 50%) in comparison to the Fstl1 expression level of a reference level, is indicative of a subject having, or likely to develop a cardiac stress and/or skeletal muscle stress.

In another embodiment of the invention, pattern comparison software is used to determine whether the Fstl1 expression level is below a threshold level (i.e. below a threshold level of at least about 30%, or at least about 40% or at least about 50%) in comparison to the Fstl1 expression level of a reference level, is indicative of a subject having, or likely to develop a metabolic dysfunction, such as diabetes or obesity.

By providing Fstl1 expression level information in computer-readable form, one can use the Fstl expression level information in readable form in the comparison module to compare a reference Fstl1 expression level information of the reference data within the storage device. For example, search programs can be used to identify relevant reference data (i.e. reference Fstl1 expression level information) that match a particular subject (reference data, e.g. data obtained from a control reference biological sample from the same subject, for example at an earlier timepoint, i.e. t₁, t₂, t₃ when comparing against a timepoint of t₄ or above) or direct comparison of the determined Fstl1 expresion level in the biological sample which can be compared to the reference data (i.e. reference Fstl1 expression level information) (e.g. data obtained from a control sample). The comparison made in computer-readable form provides computer readable content which can be processed by a variety of means. The content can be retrieved from the comparison module, the retrieved content.

In some embodiments of this aspect and all other aspects of the present invention, the “comparison module” can use a variety of available software programs and formats for the comparison operative to compare sequence information determined in the determination module to reference data. In one embodiment, the comparison module is configured to use pattern recognition techniques to compare sequence information from one or more entries to one or more reference data patterns. The comparison module may be configured using existing commercially-available or freely-available software for comparing patterns, and may be optimized for particular data comparisons that are conducted. The comparison module can also provide computer readable information related to the sequence information that can include, for example, detection of the presence or absence of a sequence (e.g., detection of a mutation or deletion (protein or DNA), information regarding distinct alleles, or omission or repetition of sequences); determination of the concentration of a sequence in the sample (e.g. amino acid sequence/protein expression levels, or nucleotide (RNA or DNA) expression levels), or determination of an expression profile.

In some embodiments, the comparison module 80 provides computer readable comparison result that can be processed in computer readable form by predefined criteria, or criteria defined by a user, to provide a content based in part on the comparison result that may be stored and output as requested by a user using a display module 110. The display module 110 enables display of a content based in part on the comparison result for the user, wherein the content 140 is a signal indicative of a subject having or at risk of having cardiac stress and/or skeletal muscle stress. In some embodiments, the display module 110 enables display of a content based in part on the comparison result for the user, wherein the content 140 is a signal indicative of a subject having or at risk of having diabetes and/or metabolic dysfunction. In some embodiments, a signal can be for example, a display of content 140 indicative of a subject having or at risk of having cardiac stress and/or skeletal muscle stress on a computer monitor, a printed page of content 140 indicating a subject having or at risk of having cardiac stress and/or skeletal muscle stress from a printer, or a light or sound indicative a subject having or at risk of having cardiac stress and/or skeletal muscle stress. Where the comparison result is indicative of a subject having or at risk of having diabetes and/or a metabolic disorder, such signal, can be for example, a display of content 140 indicative of a subject having or at risk of having diabetes and/or metabolic dysfunction on a computer monitor, a printed page of content 140 indicating the presence or absence of a subject having or at risk of having diabetes and/or metabolic dysfunction from a printer, or a light or sound indicative of a subject having or at risk of having diabetes and/or metabolic dysfunction.

In some embodiments, where the Fstl1 expression level from the biological sample is compared to a reference Fstl1 expression level from the same subject taken from the same subject at an earlier timepoint, the content 140 is a signal indicative that the subject from which the biological sample was obtained has, has an improvement or increased recovery in cardiac stress or skeletal muscle stress. Such signal, can be for example, a display of content 140 indicative an improvement or recovery in cardiac stress or skeletal muscle stress in the subject on a computer monitor, a printed page of content 140 indicating an improvement or recovery in cardiac stress or skeletal muscle stress in the subject from a printer, or a light or sound indicative of an improvement or recovery in cardiac stress or skeletal muscle stress in the subject.

The content 140 based on the comparison result may include an expression profile of one or more Fstl1 proteins, such as Fstl1 isoforms, or an expression profile of one or more Fstl1 genes. In some embodiments, the content 140 may also be based upon the presence or absence of am additional agent, for example an additional marker of cardiac stress, and/or injury or both. In one embodiment, the content 140 based on the comparison includes the presence or absence of at least one Fstl1 protein and a determination of the level of at least one Fstl1 protein, or specific post-translational modification of at least one protein. In one embodiment, the content 140 based on the comparison result is merely a signal indicative of a subject having or at risk of having cardiac stress and/or skeletal muscle stress. In one embodiment, the content 140 based on the comparison result is merely a signal indicative of the Fstl1 expression level in the biological sample above or below a threshold level, wherein the threshold level is relative to the reference Fstl1 expression level. In some embodiments, the content 140 based on the comparison result is merely a signal indicative of the Fstl1 expression level in the biological sample at least about 30% above the reference Fstl1 expression level. In some embodiments, the content 140 based on the comparison result is merely a signal indicative of the Fstl1 expression level in the biological sample at least about 30% below the reference Fstl1 expression level.

In one embodiment of the invention, the content 140 based on the comparison result is displayed a on a computer monitor. In one embodiment of the invention, the content 140 based on the comparison result is displayed through printable media. In one embodiment of the invention, the content 140 based on the comparison result is displayed as an indicator light or sound. The display module 110 can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content 140 based on the comparison result. It should be understood that other modules of the invention can be adapted to have a web browser interface. A user interface useful in various embodiments includes, for example, a display screen or a printer or other means for providing a readout of the result of a computer-mediated process. A user interface can also include, for example, an address in a network or on the world wide web to which the results of a process are transmitted and made accessible to one or more users. For example, the user interface can include a graphical user interface comprising an access element that permits entry of data regarding Fstl1 expression level in a biological sample, as well as an access element that provides a graphical read out of the results of a comparison transmitted to or made available by a processor following execution of the instructions encoded on a computer-readable medium. Through the Web browser, a user may construct requests for retrieving data from the comparison module. Thus, the user will typically point and click to user interface elements such as buttons, pull down menus, scroll bars and the like conventionally employed in graphical user interfaces. The requests so formulated with the user's Web browser are transmitted to a Web application which formats them to produce a query that can be employed to extract the pertinent information related to the Fstl1 expression level information, e.g., display of an indication of a subject having or at risk of having cardiac stress and/or skeletal muscle stress (i.e. a positive or negative cardiac stress and/or skeletal muscle test result); display of an indication of a subject having or at risk of having diabetes and/or metabolic dysfunction (i.e. a positive or negative diabetes and/or metabolic dysfunction test result); display of expression levels of the Fstl1 proteins measured; display of nucleotide (RNA or DNA) Fstl1 expression levels; display of Fstl1 protein expression as compared to reference Fstl1 level information, display of Fstl1 protein expression level for each biological sample measured selected from a variety of different biological samples (i.e. taken a different timepoints from the same subject or taken from different tissues from the same subject), or display of Fstl1 level information based thereon. In one embodiment, the Fstl1 expression level information of the reference sample data is also displayed.

In one embodiment, the display module 110 displays the comparison result and whether the comparison result is indicative a subject having or at risk of having cardiac stress and/or skeletal muscle stress (e.g. whether the expression profile of Fstl1 is indicative of whether a subject has a positive or negative cardiac stress and/or skeletal muscle test result).

In one embodiment, the display module 110 displays the comparison result and whether the comparison result is indicative a subject having or at risk of having diabetes and/or a metabolic dysfunction (e.g. whether the expression profile of Fatalism indicative of whether a subject has a positive or negative diabetes and/or a metabolic dysfunction test result).

In one embodiment, the content 140 based on the comparison result that is displayed is a signal (e.g. positive or negative signal) is indicative a subject having or at risk of having cardiac stress and/or skeletal muscle stress (e.g. whether the expression profile of Fatalism indicative of whether a subject has a positive or negative cardiac stress and/or skeletal muscle test result), thus only a positive or negative cardiac stress and/or skeletal muscle stress indication may be displayed. In an alternative embodiment, where diabetes and/or metabolic dysfunction is being assessed, the content 140 based on the comparison result that is displayed is a signal (e.g. positive or negative signal) indicative a subject having or at risk of having diabetes and/or metabolic dysfunction (e.g. whether the expression profile of Fatalism indicative of whether a subject has a positive or negative diabetes and/or metabolic dysfunction test result), thus a positive or negative diabetes and/or metabolic dysfunction be displayed.

The present invention therefore provides for systems 10 (and computer readable medium 200 for causing computer systems) to perform methods for determining the presence of cardiac stress and/or skeletal muscle stress (i.e. a positive or negative cardiac stress and/or skeletal muscle stress) or in some embodiments, whether a subject has, or will likely develop diabetes and/or metabolic dysfunction based on the Fstl1 expression level information.

System 10, and computer readable medium 200, are merely an illustrative embodiments of the invention for performing methods of determining the presence of, or risk of a subject developing cardiac stress and/or skeletal muscle stress, and in some embodiments, whether a subject has, or likely will develop diabetes and/or metabolic dysfunction, and is not intended to limit the scope of the invention. Variations of system 10, and computer readable medium 200, are possible and are intended to fall within the scope of the invention.

The modules of the system 10 or used in the computer readable medium 200, may assume numerous configurations. For example, function may be provided on a single machine or distributed over multiple machines.

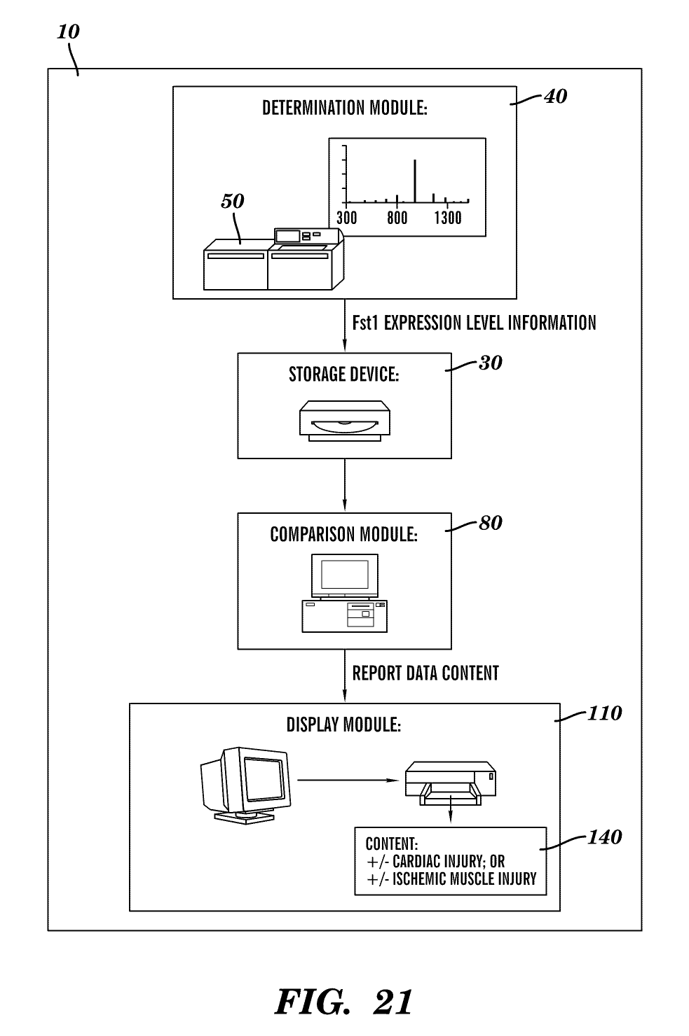

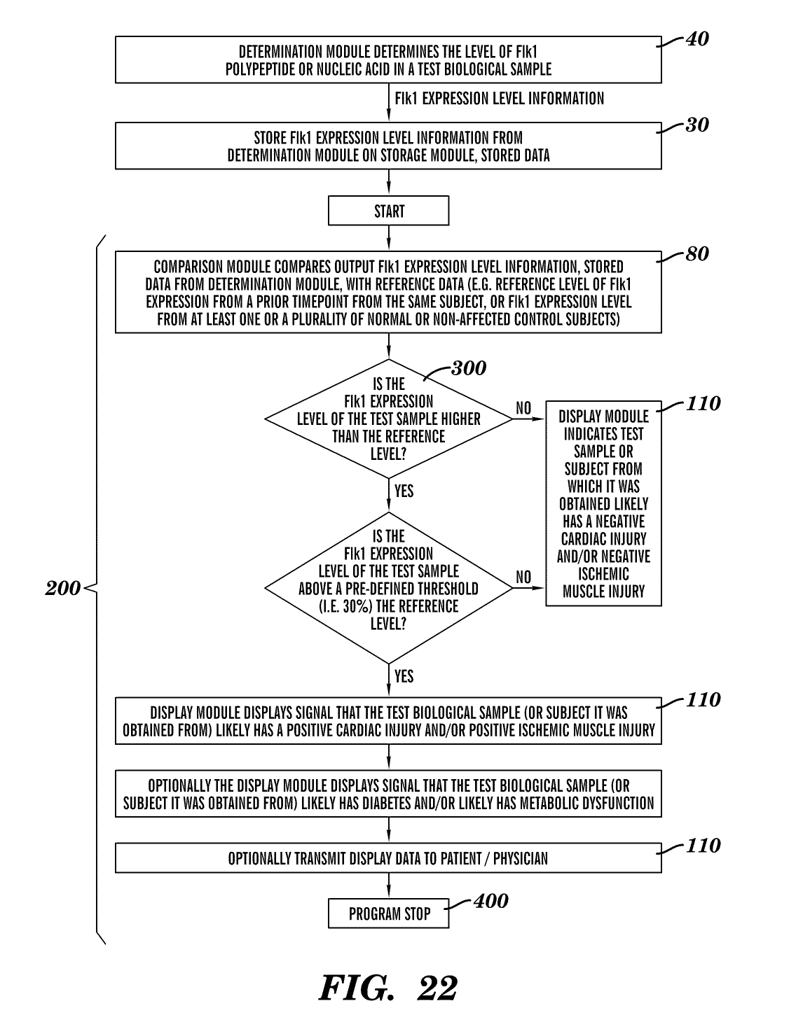

FIG. 21 is a block diagram of a computer readable media 200 according to one embodiment of the invention. The system shown in FIG. 21 for performing the comparison processing of the invention may be a general purpose computer used alone or in connection with a specialized processing computer. Such processing may be performed by a single platform or by a distributed processing platform. In addition, such processing and functionality can be implemented in the form of special purpose hardware or in the form of software being run by a general purpose computer. Any data handled in such processing or created as a result of such processing can be stored in a temporary memory, such as in the RAM of a given computer system or subsystem. In addition, or in the alternative, such data may be stored in longer-term storage devices, for example, magnetic disks, rewritable optical disks and so on.

The computer system 10 (FIG. 21) may include an operating system (e.g., UNIX) on which runs a relational database management system, a World Wide Web application, and a World Wide Web server. The software on the computer system may assume numerous configurations. For example, it may be provided on a single machine or distributed over multiple machines.

A World Wide Web browser may be used for providing a user interface. Through the Web browser, a user may construct search requests for retrieving data from a sequence database and/or a genomic database. Thus, the user will typically point and click to user interface elements such as buttons, pull down menus, scroll bars, etc. conventionally employed in graphical user interfaces. The requests so formulated with the user's Web browser are transmitted to a Web application which formats them to produce a query that can be employed to extract the pertinent information from relevant databases, e.g. reference level databases. When network employs a World Wide Web server, it supports a TCP/IP protocol. Local networks such as this are sometimes referred to as “Intranets.” An advantage of such Intranets is that they allow easy communication with public domain databases residing on the World Wide Web (e.g., the GenBank World Wide Web site). Thus, in a particular preferred embodiment of the present invention, users can directly access data (via Hypertext links for example) residing on Internet databases using a HTML interface provided by Web browsers and Web servers.

In some embodiments, a Fstl1 expression level is measured at the level of protein expression, using any method commonly known by persons of ordinary skill in the art, for example, using an antibody, antibody fragment, recombinant antibody, chimeric antibody, aptamer, peptide or analogue thereof. Alternatively, one can measure Fstl1 expression level using a method selected from the group consisting of: an immunoassay, a radioimmunoassay (RIA), an immunoradiometric assay (IRMA), an enzyme-linked immunosorbent assay (ELISA); an ELISpot; CELISA [cellular enzyme-linked immunosorbent assay]; a RHPA (reverse hemolytic plaque assay) or a kinase receptor activation assay (KIRA), or measuring the level of Fstl1 protein expression using common protein detection agents such as an antibody, humanized antibody, antibody fragment, recombinant antibody, chimeric antibody, aptamer, peptide or analogue thereof.

Typically the methods, compositions, computer systems and computable readable media as disclosed herein are useful in the diagnosis and/or determining cardiac stress and/or skeletal muscles stress in a subject, such as a mammalian subject including a human subject, however, the methods and compositions are equally applicable to non-human subjects, including livestock, domestic and companion animals, and other veterinary and wild-life subjects. Similarly, the methods, compositions, computer systems and computable readable media as disclosed herein are useful in the diagnosis and/or determining diabetes and/or metabolic dysfunction in a subject, such as a mammalian subject including a human subject, however, the methods and compositions are equally applicable to non-human subjects, including livestock, domestic and companion animals, and other veterinary and wild-life subjects.

In another embodiment, the methods, systems, compositions and computer readable media as disclosed herein are useful for predicting prognosis of cardiac stress and/or skeletal muscle stress in the subject, for example, where a subject which has an Fstl1 expression level lower (i.e. at least about 30%, or at least about 35%, 40%, 45%, 50% lower) as compared to a reference Fstl1 expression level taken from the same subject at an earlier timepoint is identified as likely to have a better prognosis as compared to a subject who is identified to the same or higher Fstl1 expression level as compared to the reference Fstl1 expression level taken from the same subject at an earlier timepoint. In another embodiment, the methods, systems, compositions and computer readable media as disclosed herein are useful for predicting the risk useful for predicting prognosis of diabetes and/or metabolic dysfunction in the subject, for example, where a subject which has an Fstl1 expression level higher (i.e. at least about 30%, or at least about 35%, 40%, 45%, 50% higher) as compared to a reference Fstl1 expression level taken from the same subject at an earlier timepoint is identified as likely to have a better prognosis as compared to a subject who is identified to have the same or lower Fstl1 expression level as compared to the reference Fstl1 expression level taken from the same subject at an earlier timepoint

### Controls

A variety of appropriate controls for the methods described herein are available for use or can otherwise be generated by the skilled practitioner. As discussed above, Fstl1 expression levels can be determined and compared to earlier determinations in the same subject to provide useful information to the skilled practitioner in diagnosis and prognosis of the individual, regarding cardiac stress, and/or injury or both, skeletal muscle stress and/or injury, and metabolic condition. Such tracking of Fstl1 expression levels in an individual can be useful for establishing a baseline and determining the progression of the individual with respect to cardiac, skeletal muscle, and metabolic condition, as it relates to the progressing health of the individual over the course of the various readings of Fstl1 expression levels. Such determinations of Fstl expression levels in a biological subject are particularly suited for tracking the progression (i.e. prognosis) or risk of a disease in an individual, and also in tracking the progression or recovery of an individual following treatment or therapy. In some embodiments, such determinations of Fstl expression levels in a biological subject are also particularly suited for monitoring disease severity of a disease in an individual (i.e. cardiac stress and/or skeletal muscle stress and/or diabetes and/or metabolic dysfunction) in an individual, prior to, during or following a treatment or therapy.

In one embodiment, a baseline or reference Fstl1 expression level can be obtained from a subject at a first timepoint (i.e. to) which can be prior to development of symptoms. In another embodiment, a of baseline Fstl1 expression (i.e. a reference Fstl1 expression level) is established after the development of symptoms (e.g. early on, midstage, in latter stages) of one or more disorder, to track the progression of that disorder(s), and possibly the development of other disorders related to an increase in Fstl1 expression level. The existence of a baseline can be useful in determining if preventative measures or existing therapies of the disease or disorder are having the desired effect in the individual. Such tracking can also indicate whether therapies or preventative measure, or other aspects of the subject's life (e.g., lifestyle) are having a negative effect in the individual. Such an indication may provide the necessary feedback, to motivate positive lifestyle choices, and/or other therapeutic intervention.

A relatively high level of Fstl1 expression level can be determined by quantitative comparison of the determined Fstl1 expression levels to the similarly obtained levels of an appropriate healthy control subject. A relatively high Fstl1 expression level may also be determined by quantitative comparison of the determined Fstl1 expression levels to a predetermined continuum compiled from a survey of a number of individuals (e.g. healthy individuals and/or individuals with one or more cardiac stress or injury described herein). For example, tracking the condition and expressed levels of Fstl1 as described herein, for numerous individuals can be performed, and the data compiled to generate a corresponding scale of Fstl1 levels regarding the stress or injury or metabolic function being assessed. As the numbers if individuals increase, the accuracy of such a control improves. Preferably, one has at least 5 individuals of similar age (within 0-10 years of one another, more preferably within 0-5 years) and sex, gender and ethnicity. Such a scale, once generated, can further be used to correlate Fstl1 expression levels in an individual with likelihood of cardiac stress and/or skeletal muscle stress/injury or metabolic dysfunction. As such, the scale would serve as an appropriate controls useful in the methods described herein.

It is expected that the precise quantitation of cardiac stress or skeletal muscle stress, and/or injury, or metabolic dysfunction, from the detected levels of Fstl1 may depend upon various factors, especially when comparing to outside controls (another subject or a predetermined continuum). Such factors may be specific to the individual (e.g. weight, age, overall health, medications or treatments undergone, prior determined Fstl1 expression levels, etc.). In some embodiments, the diagnosis can be appropriately adjusted for such factors by the skilled practitioner. It is also expected that even without careful control of such factors, quantitative detection of Fstl1 levels and/or expression in a subject, to determine relative levels of Fstl1, will produce useful information with respect to diagnosis and prognosis.

### Detection of Fstl1 Expression Levels

The Fstl1 protein or mRNA is detected from a biological sample of the test subject. In one embodiment, the biological sample contains protein molecules from the test subject. Biological samples which are at or near to the site of the assessed tissue injury or stress, or are known by the skilled practitioner to receive secreted proteins from such a site (e.g., serum, blood, plasma, lymph) are useful in the invention.

Alternatively, the biological sample can contain mRNA molecules from the test subject. Examples of such useful biological samples are tissue which are being assessed for stress/injury, as well as tissues and/or cells located directly adjacent the damaged tissues. Without limitation, the term “biological sample” includes blood, serum, plasma, sputum, spinal fluid, pleural fluid, nipple aspirates, external secretions of the skin, respiratory, intestinal, and genitourinary tracts, saliva, stool, tissue, urine, stool, lymph fluid, tears, and milk. In one embodiment, the tissue is from an organ, skeletal muscle, neuronal tissue, cardiac tissue. In one embodiment, the organ is the heart. In one embodiment, the tissue is cardiac muscle. In one embodiment, the tissue is obtained through a biopsy.

The complete codons and amino acid sequence of human Fstl1 are known in the art, and available to the skilled artisan (e.g., accession AK31226). Mouse, rat, and cow Fstl1 protein and mRNA sequence are also known (e.g., accession NM_{—}008047, NM 024369, and BC 114758, respectively). The domain structure of Fstl1 has been determined by sequence comparisons. The protein has a Follistatin N-terminal domain-like structure at amino acids 29-52 (note: amino acid residue numbers are for the bovine polypeptide (GenBank Accession No. NP_{—}001017950)—alignment with the human polypeptide (GenBank Accession No. AAH00055) permits delineation of corresponding domains in the human polypeptide), a Kazal serine protease inhibitor domain at amino acids 52-97, a low complexity region between amino acids 102-111, a region with insignificant similarity to other domains at amino acids 112-146, two EF Hand calcium-binding like domains at residues 147-174 and 196-224, and a coiled coil domain at residues 269-302. Useful portions of the polypeptide can include, for example, one or more of these structural domains, e.g., a Follistatin-like N terminal domain, a Kazal serine protease inhibitor-like domain, or an EF Hand domain. Other examples include two or more of these domain structures, e.g., a Follistatin-like N terminal domain and a Kazal serine protease inhibitor-like domain, a Kazal serine protease inhibitor-like domain and an EF Hand domain, two EF Hand domains, a Kazal serine protease inhibitor-like domain and two EF Hand domains, an EF Hand domain and a coiled coil, etc. Protease inhibiting or calcium binding activity can be assayed for a given construct according to methods known in the art. The function of a given portion, fragment or variant can be assayed by introducing the portion or fragment to skeletal muscle cells in culture and assaying for one or more effects of the wild-type Fstl1 polypeptide, e.g., changes in phosphorylation of AMP-activated protein kinase and/or ACC, or changes in levels of PGC-1-a or Glut4 in C2C12 cells. Alternatively, effects of a given fragment or portion can be monitored by administering the polypeptide to, or expressing the polypeptide in ob/ob mice and measuring impact on glucose tolerance. As another alternative, any of the other assays described in the Examples herein can be used to monitor the activity of a given fragment, variant, mutein or derivative of the Fstl1 polypeptide.

Quantitative detection of Fstl1 expression levels can be accomplished by quantitative detection of Fstl1 mRNA or the expressed protein or a polypeptide fragment thereof. Detection of mRNA is accomplished through a wide variety of methods known in the art. For example, Northern hybridizations, in situ hybridizations, and quantitative PCR methods such as RT-PCR.

Fstl1 protein or polypeptide expression levels can be detected by immunoassays, or by other such binding assays using a molecule which specifically binds to Fstl1 (e.g., a receptor molecule) or a “protein-binding molecule”. Antibodies to anti-Fstl polypeptide can be used in the methods, systems and computer readable media as disclosed herein. Antibodies specific to human Fstl1 polypeptide are commercially available, for example, from Abcam, Acris, santa Cruz Biotechnology, Sigma to name a few, as well as disclose in U.S. Patent Application US2007/0253962, which is specifically incorporated herein in its entirety by reference.

In some embodiments, assays based on Fstl1 activity (e.g. as an anti-apoptotic protein) can be used. Fstl1 has been shown to be post-translationally modified. The secreted form of Fstl1 has a larger molecular weight. Without being bound by theory, it is thought that this modification is glycoslylation of the protein. The present invention encompasses detection of one or more such modified Fstl1 proteins. For example, the 37 kDa or 46 kDa Fstl1.

Immunoassays are techniques known in the art, and include, for example, radioimmunoas say, ELISA (enzyme-linked immunosorbant assay), “sandwich” immunoas says, immunoradiometric assays, immunodiffusion assays, in situ immunoassays (using colloidal gold, enzyme or radioisotope labels, for example), western blot analysis, immunoprecipitations, immunofluorescence assays, immunoelectrophoresis assays, fluoroimmunoassay (FiA), immunoradiometric assay (IRMA), immunoenzymometric assay (IEMA), immunoluminescence assay and immunofluorescence assay (Madersbacher S, Berger P. Antibodies and immunoassays. Methods 2000;21:41-50).

The term “biological sample” is intended to include tissues, cells and biological fluids isolated from a subject, as well as tissues, cells and fluids present within a subject. That is, the methods described herein may utilize detection of Fstl1 protein, in a biological sample in vitro as well as in vivo. For example, in vitro techniques for detection of Fstl1 mRNA include Northern hybridizations and in situ hybridizations. In vitro techniques for detection of Fstl1 protein include enzyme linked immunosorbent assays (ELISAs), Western blots, immunoprecipitations, and immunofluorescence. Furthermore, in vivo techniques for detection of Fstl1 protein include introducing into a subject a labeled anti-Fstl1 protein antibody. For example, the antibody can be labeled with a radioactive marker whose presence and location in a subject can be detected by standard imaging techniques.

Accordingly, in all aspects of the present invention, the level of Fstl1 polypeptide can be determined can be using an to a protein-binding agent, also referred to herein as “protein-binding entity” or an “affinity reagent” can be used, in particular, antibodies. For instance, the affinity reagents, in particular, antibodies such as anti-Fstl1 antibodies can be used in an immunoassay, particularly in an ELISA (Enzyme Linked Immunosorbent Assay). In embodiments where the level of Fstl1 polypeptide can be measured in a biological sample using methods commonly known in the art, and include, for example but not limited to; isoform-specific chemical or enzymatic cleavage of isoform proteins, immunobloting, immunohistochemical analysis, ELISA, and mass spectrometry.

As mentioned above, level of Fstl1 polypeptide can be detected by immunoassays, such as enzyme linked immunoabsorbant assay (ELISA), radioimmunoassay (RIA), Inmunoradiometric assay (IRMA), Western blotting, immunocytochemistry or immunohistochemistry, each of which are described in more detail below. Inmunoassays such as ELISA or RIA, which can be extremely rapid, are more generally preferred. Antibody arrays or protein chips can also be employed, see for example U.S. Patent Application Nos: 20030013208A1; 20020155493A1; 20030017515 and U.S. Pat. Nos: 6,329,209; 6,365,418, which are herein incorporated by reference in their entirety.

Immunoassays

The most common enzyme immunoassay is the “Enzyme-Linked Immunosorbent Assay (ELISA).” ELISA is a technique for detecting and measuring the concentration of an antigen using a labeled (e.g. enzyme linked) form of the antibody. There are different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. 1984, J. Clin. Chem. Clin. Biochem., 22:895-904.

In a “sandwich ELISA”, an antibody (e.g. anti-enzyme) is linked to a solid phase (i.e. a microtiter plate) and exposed to a biological sample containing antigen (e.g. enzyme). The solid phase is then washed to remove unbound antigen. A labeled antibody (e.g. enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and B-galactosidase. The enzyme linked antibody reacts with a substrate to generate a colored reaction product that can be measured.

In a “competitive ELISA”, antibody is incubated with a sample containing antigen (i.e. enzyme). The antigen-antibody mixture is then contacted with a solid phase (e.g. a microtiter plate) that is coated with antigen (i.e., enzyme). The more antigen present in the sample, the less free antibody that will be available to bind to the solid phase. A labeled (e.g., enzyme linked) secondary antibody is then added to the solid phase to determine the amount of primary antibody bound to the solid phase.

In an “immunohistochemistry assay” a section of tissue is tested for specific proteins by exposing the tissue to antibodies that are specific for the protein that is being assayed. The antibodies are then visualized by any of a number of methods to determine the presence and amount of the protein present. Examples of methods used to visualize antibodies are, for example, through enzymes linked to the antibodies (e.g., luciferase, alkaline phosphatase, horseradish peroxidase, or beta-galactosidase), or chemical methods (e.g., DAB/Substrate chromagen). The sample is then analysed microscopically, most preferably by light microscopy of a sample stained with a stain that is detected in the visible spectrum, using any of a variety of such staining methods and reagents known to those skilled in the art.

Alternatively, “Radioimmunoassays” can be employed. A radioimmunoassay is a technique for detecting and measuring the concentration of an antigen using a labeled (e.g., radioactively or fluorescently labeled) form of the antigen. Examples of radioactive labels for antigens include 3H, 14C, and 125I. The concentration of antigen enzyme in a biological sample is measured by having the antigen in the biological sample compete with the labeled (e.g. radioactively) antigen for binding to an antibody to the antigen. To ensure competitive binding between the labeled antigen and the unlabeled antigen, the labeled antigen is present in a concentration sufficient to saturate the binding sites of the antibody. The higher the concentration of antigen in the sample, the lower the concentration of labeled antigen that will bind to the antibody.

In a radioimmunoassay, to determine the concentration of labeled antigen bound to antibody, the antigen-antibody complex must be separated from the free antigen. One method for separating the antigen-antibody complex from the free antigen is by precipitating the antigen-antibody complex with an anti-isotype antiserum. Another method for separating the antigen-antibody complex from the free antigen is by precipitating the antigen-antibody complex with formalin-killed S. aureus. Yet another method for separating the antigen-antibody complex from the free antigen is by performing a “solid-phase radioimmunoassay” where the antibody is linked (e.g., covalently) to Sepharose beads, polystyrene wells, polyvinylchloride wells, or microtiter wells. By comparing the concentration of labeled antigen bound to antibody to a standard curve based on samples having a known concentration of antigen, the concentration of antigen in the biological sample can be determined.

An “Immunoradiometric assay” (IRMA) is an immunoassay in which the antibody reagent is radioactively labeled. An IRMA requires the production of a multivalent antigen conjugate, by techniques such as conjugation to a protein e.g., rabbit serum albumin (RSA). The multivalent antigen conjugate must have at least 2 antigen residues per molecule and the antigen residues must be of sufficient distance apart to allow binding by at least two antibodies to the antigen. For example, in an IRMA the multivalent antigen conjugate can be attached to a solid surface such as a plastic sphere. Unlabeled “sample” antigen and antibody to antigen which is radioactively labeled are added to a test tube containing the multivalent antigen conjugate coated sphere. The antigen in the sample competes with the multivalent antigen conjugate for antigen antibody binding sites. After an appropriate incubation period, the unbound reactants are removed by washing and the amount of radioactivity on the solid phase is determined. The amount of bound radioactive antibody is inversely proportional to the concentration of antigen in the sample.

Other techniques can be used to detect the level of Fstl1 polypeptide in a biological sample can be performed according to a practitioner's preference, and based upon the present disclosure and the type of biological sample (i.e. plasma, urine, tissue sample etc). One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Detectably labeled anti-Fstl1 antibodies or protein binding molecules can then be used to assess the level of Fstl1 polypeptide, where the intensity of the signal from the detectable label corresponds to the amount of Fstl1 polypeptide. Levels of the amount of the Fstl1 polypeptide present can also be quantified, for example by densitometry.

In one embodiment, the level of Fstl1 polypeptide in a biological sample can be determined by mass spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

In particular embodiments, these methodologies can be combined with the machines, computer systems and media to produce an automated system for determining the level of Fstl1 polypeptide in a biological sample and analysis to produce a printable report which identifies, for example, the level of level of Fstl1 polypeptide in a biological sample.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. See, e.g., U.S. Pat. No. 5,118,937 (Hillenkamp et al.), and U.S. Pat. No. 5,045,694 (Beavis & Chait) which are incorporated herein by reference.

In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 and WO 98/59361 which are incorporated herein by reference. The two methods can be combined by, for example, using a SELDI affinity surface to capture an analyte and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094.

Detection of the presence or level of Fstl1 polypeptide will typically depend on the detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of particular biomolecules. Software programs such as the Biomarker Wizard program (Ciphergen Biosystems, Inc., Fremont, Calif.) can be used to aid in analyzing mass spectra. The mass spectrometers and their techniques are well known to those of skill in the art.

In some embodiment of this aspect and all aspects disclosed herein, a biological sample can be monitored using radioactive labeling, in particular, to an inverse radioactive labeling, preferably with iodine isotopes. Preferably, an inverse radioactive labeling is performed using ¹²⁵I and ¹³¹I isotopes. In another embodiment, a subject, for example a human subject can be subjected to a radioactive labeling, in particular, to an inverse radioactive labeling, preferably with iodine isotopes, such as but not limited to ¹²⁵I and ¹³¹I isotopes.

In all aspects of the present invention, level of Fstl1 polypeptide can be determined based on gel electrophoresis techniques, in particular SDS-PAGE (Sodium Dodecylsulfate Polyacrylamide Gel Elektrophoresis), especially two dimensional PAGE (2D-PAGE), preferably two dimensional SDS-PAGE (2D-SDS-PAGE). According to a particular example, the assay is based on 2D-PAGE, in particular, using immobilized pH gradients (IPGs) with a pH range preferably over pH 4-9.

In all aspects of the present invention, level of Fstl1 polypeptide can be determined can be using gel electrophoresis techniques, in particular, the above mentioned techniques may be combined with other protein separation methods, particularly methods known to those skilled in the art, in particular, chromatography and/or size exclusion. In all aspects of the present invention, the level of Fstl1 polypeptide can be determined, if appropriate, using a combination of any of the above mentioned methods with a combination of detection methods which are well known to those skilled in the art, such as, but not limited to antibody detection and/or mass spectrometry.

In a further embodiment of all aspects of the present invention, the level of Fstl1 polypeptide can be determined can be using mass spectrometry as disclose herein in the Examples, and in particular, MALDI (Matrix Assisted Laser Desorption/Ionization) and/or SELDI (Surface enhanced Laser Desorption/Ionization). In an alternative embodiment, resonance techniques, in particular, plasma surface resonance, can be used.

In some cases, it may be advantageous to achieve a separation of Fstl1 polypeptides from a heterogeneous population of proteins in a biological sample for example using a means of one of the above outlined methods before cleaving the proteins. Such a cleavage step can be performed by applying enzymes, chemicals or other suitable reagents which are known to those skilled in the art. In an alternative embodiment, one may perform a cleavage step and subsequent separation of the cleaved Fstl1 polypeptide fragments, in particular, followed by, for example, measurements of the level Fstl1 polypeptide using any one of the methods, kits, machines, computer systems or media as disclosed herein. In some embodiments of this aspect of the invention, the cleaved Fstl1 polypeptide fragments can be labeled and, optionally separated where the protein spots which correspond to cleaved Fstl1 polypeptide fragments can be visualized by imaging techniques, for instance using the PROTEP TOPO® imaging technique.

In some embodiments, a protein-binding agents or antibodies or useful in the methods as disclosed herein bind or have affinity for the Fstl1 polypeptide.

In some embodiments, protein-binding moieties such as antibodies can be utilized to detect the level of Fstl1 polypeptide by itself (i.e. individually), or when the Fstl1 polypeptide exists in complex with other polypeptides. Additionally, in other embodiments, protein-binding moieties such as antibodies can be utilized to detect the presence of Fstl1 polypeptide when it is post-translationally modified, for example when Fstl1 polypeptide is ubiquitiniated. In some embodiments, protein binding moieties such as antibodies can bind to Fstl1 polypeptide individually or in a complex, and in some embodiments a protein-binding moiety such as an antibody can be labeled with a detectable label.

In some embodiments, antibodies and protein-binding molecules are labeled. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin.

In all aspects of the present invention, the level of a Fstl1 polypeptide can be determined by using immunological techniques using an anti-Fstl1 antibody, using common methods known by a person of ordinary skill in the art, e.g., antibody techniques such as immunohistochemistry, immunocytochemistry, FACS scanning, immunoblotting, radioimmunoassays, western blotting, immunoprecipitation, enzyme-linked immunosorbant assays (ELISA), and derivative techniques that make use of antibodies directed against the Fstl1 polypeptide, or variants or derivatives thereof.

Any method to detect a Fstl1 polypeptide known by a person of ordinary skill in the art are useful in the methods, kits, machines and computer systems and media as disclosed herein to detect the level of Fstl1 polypeptide. For example, immunohistochemistry (“IHC”) and immunocytochernistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of a specific antibody, wherein antibodies are used to specifically target molecules inside or on the surface of cells. The antibody typically contains a marker that will undergo a biochemical reaction, and thereby experience a change color, upon encountering the targeted molecules. In some instances, signal amplification may be integrated into the particular protocol, wherein a secondary antibody, that includes the marker stain, follows the application of a primary specific antibody.

Immunohistochemical assays are well known to those of skill in the art (e.g., see Jalkanen, et al., J. Cell. Biol. 101:976-985 (1985); Jalkanen, et al., J. Cell. Biol. 105:3087-3096 (1987).

In some embodiments, antibodies, polyclonal, monoclonal and chimeric antibodies useful in the methods as disclosed herein can be purchased from a variety of commercial suppliers, or may be manufactured using well-known methods, e. g., as described in Harlow et al., Antibodies: A Laboratory Manual, 2nd Ed; Cold. Spring Harbor Laboratory Press, Cold Spring Harbor, N.Y. (1988). In general, examples of antibodies useful in the present invention include anti-serine antibodies. Such antibodies can be purchased, for example, from Sigma-Aldrich, CalBiochem, Abcam, Santa-Cruz Biotechnology, novus Bio, U.S. biologicals, Millipore, LifeSpan, Abnova, CellSignalling etc.

Typically, for immunohistochemistry, tissue obtained from a subject and fixed by a suitable fixing agent such as alcohol, acetone, and paraformaldehyde, is sectioned and reacted with an antibody. Conventional methods for immunohistochemistry are described in Harlow and Lane (Eds) (1988) In “Antibodies A Laboratory Manual”, Cold Spring Harbor Press, Cold Spring Harbor, N.Y.; Ausbel et al (Eds) (1987), in Current Protocols In Molecular Biology, John Wiley and Sons (New York, N.Y.). Biological samples appropriate for such detection assays include, but are not limited to, cells, tissue biopsy, whole blood, plasma, serum, sputum, cerebrospinal fluid, breast aspirates, pleural fluid, urine and the like.

In some embodiments, direct labeling techniques can be used, where a labeled antibody is utilized. For indirect labeling techniques, the sample is further reacted with a labeled substance.

In some embodiments, immunocytochemistry may be utilized where, in general, tissue or cells are obtained from a subject are fixed by a suitable fixing agent such as alcohol, acetone, and paraformaldehyde, to which is reacted an antibody. Methods of immunocytological staining of human samples is known to those of skill in the art and described, for example, in Brauer et al., 2001 (FASEB J, 15, 2689-2701), Smith Swintosky et al., 1997.

Immunological methods are particularly useful in the methods as disclosed herein, because they require only small quantities of biological material, and are easily performed and at multiple different locations. In some embodiments, such an immunological method useful in the methods as disclosed herein uses a “lab-on-a-chip” device, involving a single device to run a single or multiple biological samples and requires minimal reagents and apparatus and is easily performed, making the “lab-on-a-chip” devices which detect the Fstl1 polypeptide is ideal for rapid, on-site diagnostic tests to identify if a biological sample obtained from a subject is likely to be at risk of cardiac stress or skeletal muscle stress and/or diabetes and/or metabolic dysfunction. In some embodiments, the immunological methods can be done at the cellular level and thereby necessitate a minimum of one cell. Preferably, several cells are obtained from a subject affected at risk for cardiac stress, or following an injury such as a cardiac injury or ischemic injury using the methods, computer systems and computer readable media as disclosed herein.

### Detectable Stress, Injuries, and Conditions

Cardiac and skeletal muscle stress which can lead to detectable injury can be identified by the methods of the present invention. Upon detectable injury, the stress is often ongoing. As such, detection of stress and injury may be co-extensive. Levels of detection that are relatively subtle may indicate stress in the absence of injury, mild injury, or ongoing progression of recovery from an injury.

Cardiac muscle injury (heart injury) includes any injury to cardiac muscle that results from or leads to loss of adequate blood flow to the heart. This can result from, or be associated with, the various known heart diseases and heart conditions, as well as injuries to the heart resulting from cardiac trauma. Heart disease is a general term that refers to any disease or condition of the heart, including coronary heart disease, hypertension, heart failure, myocardial infarction, congenital heart disease, disorders of the heart valves, heart infections, cardiomyopathy, conduction disorders, and heart arrhythmias. Cardiac injury can result from a variety of traumas, no limited to penetrating cardiac trauma, blunt cardiac trauma, iatrogenic cardiac injury, metabolic cardiac injury, and electrical injury.

A heart attack (also known as a myocardial infarction) is the death of heart muscle from the sudden blockage of a coronary artery by a blood clot. Coronary arteries are blood vessels that supply the heart muscle with blood and oxygen. Blockage of a coronary artery deprives the heart muscle of blood and oxygen, causing injury to the heart muscle. Injury to the heart muscle causes chest pain and pressure. If blood flow is not restored within 20 to 40 minutes, irreversible death of the heart muscle will begin to occur. Muscle continues to die for six to eight hours at which time the heart attack usually is “complete.” The dead heart muscle is replaced by scar tissue.

Cardiac hypertrophy is a universal response of the heart to injury or overload, which according to the Law of Laplace acts to normalize increased wall stress. Initially described as a beneficial and necessary response to maintain cardiac output under conditions of overload, hypertrophy has more recently been recognized as a risk factor for increased mortality. A large number of studies now collectively demonstrate that modulation of the hypertrophic growth of the heart is feasible without provoking hemodynamic compromise, despite increased wall stress. In both animal studies and clinical trials, inhibition of hypertrophic growth usually results in the amelioration of LV dysfunction. Therefore, a concept of continuous progression from compensated hypertrophy toward heart failure has recently led to proposing cardiac hypertrophy as an early therapeutic target.

Conditions relating to skeletal muscle stress and injury include, without limitation, muscular dystrophy, muscle atrophy, sarcopenia, inclusion body myocytis, cardiac cachexia and cancer cachexia. Examples of muscular dystrophies include Duchene Muscular Dystrophy, Becker Muscular Dystrophy, Limb Girdle Muscular Dystrophy, Facioscapulohumeral Muscular Dystrophy, Oculopharyngeal muscular dystrophy, Emery-Dreifuss muscular dystrophy, Fukuyama-type congenital muscular dystrophy, Miyoshi myopathy, Ullrich congenital muscular dystrophy, Steinert Muscular Dystrophy.

Conditions which result from or otherwise relate to metabolic dysfunction include, without limitation, diabetes, obesity, and metabolic syndrome. Metabolic syndrome is a grouping of traits and medical conditions that puts people at risk for both heart disease and type 2 diabetes. It is defined by the National Cholesterol Education Program as having any three of the following five traits and medical conditions: Elevated waist circumference (waist measurement of 40 inches or more in men, 35 inches or more in women); Elevated levels of triglycerides (150 mg/dL or higher or taking medication for elevated triglyceride levels); low levels of HDL (good) cholesterol (below 40 mg/dL in men, below 50 mg/dL in women, or taking medication for low HDL cholesterol levels); elevated blood pressure levels (130 mm Hg or higher for systolic blood pressure, or 85 mm Hg or higher for diastolic blood pressure, or taking medication for elevated blood pressure levels); elevated fasting blood glucose levels (100 mg/dL or higher, or taking medication for elevated blood glucose levels) (Grundy S M, et al. Diagnosis and Management of the Metabolic Syndrome: An American Heart Association/National Heart, Lung, and Blood Institute Scientific Statement. Circulation. 2005;1 12:2735-2752). Other definitions of similar conditions have been developed by the American Association of Clinical Endocrinologists, the International Diabetes Federation, and the World Health Organization.

### Other Therapeutic Agents

The methods of the invention may be performed following or prior to administration of other relevant therapeutic agents. In some embodiments, where the methods and systems inducate a subject is at risk of cardiac stress, the subject can be administered a cardioprotective agent. Agents for reducing the risk of myocardial disorder include, without limitation, anti-inflammatory agents, anti-thrombotic agents and/or fibrinolytic agents, anti-platelet agents, lipid reducing agents, direct thrombin inhibitors, and glycoprotein IIb/IIIa receptor inhibitors and agents that bind to cellular adhesion molecules and inhibit the ability of white blood cells to attach to such molecules (e.g. anti-cellular adhesion molecule antibodies). One agent which may be used to reduce the risk of a future myocardial disorder in an individual testing positive for antibodies to a cardiac troponin is aspirin.

A number of anti-inflammatory agents are known in the art, non-limiting examples of which are Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone, Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Momiflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Salycilates; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Glucocorticoids; Zomepirac Sodium.

Anti-thrombotic and/or fibrinolytic agents include Plasminogen (to plasmin via interactions of prekallikrein, kininogens, Factors XII, XIIIa, plasminogen proactivator, and tissue plasminogen activator[TPA]) Streptokinase; Urokinase: Anisoylated Plasminogen-Streptokinase Activator Complex; Pro-Urokinase; (Pro-UK); rTPA (alteplase or activase; r denotes recombinant), rPro-UK; Abbokinase; Eminase; Sreptase Anagrelide Hydrochloride; Bivalirudin; Dalteparin Sodium; Danaparoid Sodium; Dazoxiben Hydrochloride; Efegatran Sulfate; Enoxaparin Sodium; Ifetroban; Ifetroban Sodium; Tinzaparin Sodium; retaplase; Trifenagrel; Warfarin; Dextrans.

Anti-platelet agents include Clopridogrel; Sulfinpyrazone; Aspirin; Dipyridamole; Clofibrate; Pyridinol Carbamate; PGE; Glucagon; Antiserotonin drugs; Caffeine; Theophyllin Pentoxifyllin; Ticlopidine; Anagrelide. Lipid reducing agents include gemfibrozil, cholystyramine, colestipol, nicotinic acid, probucol lovastatin, fluvastatin, simvastatin, atorvastatin, pravastatin, cirivastatin. Direct thrombin inhibitors include hirudin, hirugen, hirulog, agatroban, PPACK, thrombin aptamers. Glycoprotein IIb/IIIa receptor Inhibitors are both antibodies and non-antibodies, and include but are not limited to ReoPro (abciximab), lamifiban, tirofiban.

### Markers of Stress/Injury/Disease

The efficacy of cardiac protection or therapy according to the methods described herein can be evaluated by following surrogate or indirect markers of cardiac health and function. For example, cardiac cell death, whether by necrosis or apoptosis, is generally accompanied by the release of cardiac enzymes, including cardiac creatine kinase (CK). Assays for cardiac enzymes are routinely used in the diagnosis of myocardial infarction and can be used to monitor the efficacy of cardiac protection according to the methods described herein. A decrease in cardiac enzymes (e.g., a 10% or greater decrease), or a lower level than is normally seen with an infarct of a given size, is indicative of effective treatment. Other markers include, for example, cardiac Troponin T (TnT), which is a marker of cardiac injury that is used as an alternative for CK. In addition, one can perform “echocardiographic analysis of ejection fraction” as a measure of cardiac injury, stabilization after injury or recovery after injury.

Another marker of the efficacy of cardioprotective intervention as described herein is survival. Statistical survival rates for myocardial ischemic events are well established—when an individual or group of individuals treated according to the methods described herein survives beyond the expected time or at a greater than expected rate for an infarct of a given size and location, the treatment can be considered effective.

Other markers that can be determined in addition to Fstl1 include, one or more cardiac troponins, a natriuretic peptide or a natriuretic peptide-related marker, an inflammation marker, D-dimer, cholesterol, homocysteine, adiponectin, sCD40L, myeloperoxidase, and ischemia modified albumin, markers of acute inflammation and so-called proximal inflammatory markers.

Acute inflammatory markers known to the person skilled in the art include C-reactive protein (CRP), fibrinogen, D-dimer, serum amyloid A (SAA), pregnancy-associated polypeptide A (PAPP-A), intercellular adhesion molecules (e.g. ICAM-1, VCAM-1), IL-1-beta, IL-6, IL-18/IL-18b; TNF-alpha; myeloperoxidase (MPO); TF; monocyte chemoattractant protein 1 (MCP-1); P-selectin; E-selectin; platelet activating factor acetyl hydrolase (PAF-AH); von Willebrand Factor (vWF). Preferred markers of acute inflammation for use in a method according to the present invention are CRP, fibrinogen, D-dimer and SAA, of which CRP and D-dimer are more preferably used. Proximal inflammatory markers are macromolecules situated upstream, i.e. close to or at the ethiopathogenetic origin of the disease event. In particular, they are produced at the site of the coronary heart lesion, preferably at the site of an arterial plaque. Proximal inflammatory markers are in particular associated with the risk that plaques already present in an individual will undergo inflammation, or growth, and with the probability of plaque rupture and thrombus formation.

Proximal inflammatory markers are known to the person skilled in the art, and non-limiting examples include pregnancy-associated polypeptide A (PAPP-A), matrix metalloproteinases (MMPs, e.g. MMP-1,-2,-3,-4,-5,-6,-7,-9,-10,-11, -12) and lipoprotein-associated phospholipase A2 (Lp-PLA2).

The skilled artisan will appreciate that there are many ways to use the measurements of two or more markers in order to improve the diagnostic question under investigation. In a quite simple, but nonetheless often effective approach, a positive result is assumed if a sample is positive for at least one of the markers investigated. This may e.g. be the case when diagnosing an infectious disease, like AIDS, by either detecting a nucleic acid or a polypeptide of the infectious agent or by detecting antibodies to the infectious agent. Frequently, however, the combination of markers is mathematically/statistically evaluated. Preferably the values measured for markers of a marker panel, e.g. an antibody to a cardiac troponin and the level of a cardiac troponin, are mathematically combined and the combined value is correlated to the underlying diagnostic question. Preferably the diagnostic question is the relative risk of developing a myocardial disorder in the future. Preferably the relative risk is given in comparison to healthy controls. Preferably healthy controls are matched for age and other covariates.

Marker values may be combined by any appropriate state of the art mathematical method. Well-known mathematical methods for correlating a marker combination to a disease or to the risk of developing a disease employ methods like, Discriminant analysis (DA) (i.e. linear-, quadratic-, regularized-DA), Kernel Methods (i.e. SVM), Nonparametric Methods (i.e. k-Nearest-Neighbor Classifiers), PLS (Partial Least Squares), Tree-Based Methods (i.e. Logic Regression, CART, Random Forest Methods, Boosting/Bagging Methods), Generalized Linear Models (i.e. Logistic Regression), Principal Components based Methods (i.e. SIMCA), Generalized Additive Models, Fuzzy Logic based Methods, Neural Networks and Genetic Algorithms based Methods. The skilled artisan will have no problem in selecting an appropriate method to evaluate a marker combination of the present invention. Preferably the method used in correlating the marker combination of the invention e.g. to the absence or presence of myocardial disease is selected from DA (i.e. Linear-, Quadratic-, Regularized Discriminant Analysis), Kernel Methods (i.e. SVM), Nonparametric Methods (i.e. k-Nearest-Neighbor Classifiers), PLS (Partial Least Squares), Tree-Based Methods (i.e. Logic Regression, CART, Random Forest Methods, Boosting Methods), or Generalized Linear Models (i.e. Logistic Regression). Details relating to these statistical methods are found in the following references: Ruczinski, I., J. of Computational and Graphical Statistics, 12 (2003) 475-511; Friedman, J. H., Regularized Discriminant Analysis, JASA 84 (1989) 165-175; Hastie, T., Tibshirani, R., Friedman, J., The Elements of Statistical Learning, Springer Series in Statistics, 2001; Breiman, L., Friedman, J. H., Olshen, R. A., Stone, C. J., (1984) Classification and regression trees, California: Wadsworth; Breiman, L. Random Forests, Machine Learning, 45 (2001) 5-32; Pepe, M. S., The Statistical Evaluation of Medical Tests for Classification and Prediction, Oxford Statistical Science Series, 28 (2003) and Duda, R. O., Hart, P. E., Stork, D. G., Pattern Classification, Wiley Interscience, 2nd Edition (2001).

It is a preferred embodiment of the invention to use an optimized multivariate cut-off for the underlying combination of biological markers and to e. g. discriminate patients with low, intermediate and high risk of developing a myocardial disorder. In this type of multivariate analysis the markers are no longer independent but form a marker panel.

Accuracy of a diagnostic method is best described by its receiver-operating characteristics (ROC) (see especially Zweig, M. H., and Campbell, G., Clin. Chem. 39 (1993) 561-577). The ROC graph is a plot of all of the sensitivity/specificity pairs resulting from continuously varying the decision thresh-hold over the entire range of data observed.

The clinical performance of a laboratory test depends on its diagnostic accuracy, or the ability to correctly classify subjects into clinically relevant subgroups. Diagnostic accuracy measures the test's ability to correctly distinguish two different conditions of the subjects investigated. Such conditions are for example health and disease or benign versus malignant disease, respectively.

In each case, the ROC plot depicts the overlap between the two distributions by plotting the sensitivity versus 1-specificity for the complete range of decision thresholds. On the y-axis is sensitivity, or the true-positive fraction [defined as (number of true-positive test results)/(number of true-positive+number of false-negative test results)]. This has also been referred to as positivity in the presence of a disease or condition. It is calculated solely from the affected subgroup. On the x-axis is the false-positive fraction, or 1-specificity [defined as (number of false-positive results)/(number of true-negative+number of false-positive results)]. It is an index of specificity and is calculated entirely from the unaffected subgroup. Because the true- and false-positive fractions are calculated entirely separately, by using the test results from two different subgroups, the ROC plot is independent of the prevalence of disease in the sample. Each point on the ROC plot represents a sensitivity/1-specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. (If the ROC plot falls completely below the 45° diagonal, this is easily remedied by reversing the criterion for “positivity” from “greater than” to “less than” or vice versa.) Qualitatively, the closer the plot is to the upper left corner, the higher the overall accuracy of the test.

One convenient goal to quantify the diagnostic accuracy of a laboratory test is to express its performance by a single number. The most common global measure is the area under the ROC plot. By convention, this area is always >0.5 (if it is not, one can reverse the decision rule to make it so). Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area=i.0).

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means ±1%.

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

As used throughout, the terms “subject”, “individual”, “person”, “patient”, etc. are used interchangeably herein.

The present invention may be defined in any one of the following numbered paragraphs.

1. A system for analyzing a biological sample from a subject comprising:

a) a determination module configured to receive a biological sample and to determine Fstl1 expression level information, wherein the Fstl1 expression level information, comprises:

(i) the Fstl1 polypeptide expression level in the biological sample; and/or

(ii) the Fstl1 gene expression level in the biological sample;

b) a connection from the determination module to transmit the Fstl1 expression level information to an electronic computer, wherein the computer comprises a storage device, a comparison module and a display module;

c) the storage device configured to store Fstl1 expression level information from the determination module;

d) the comparison module adapted to compare the Fstl1 expression level information stored on the storage device with reference data, and to provide a comparison result, wherein the comparison result comprises;
(i) a comparison of the Fstl1 expression level in the biological sample with the reference Fstl1 expression level, and
(ii) a determination of the Fstl1 expression level in the biological sample above or below a threshold level relative to the reference Fstl1 expression level, wherein a Fstl1 expression level above the threshold level is indicative of cardiac stress and/or skeletal muscle stress; and wherein a Fstl1 expression level below the threshold level is indicative of diabetes; and/or metabolic disorder;

e) the display module for displaying a content based in part on the comparison result for the user, wherein the content is a signal indicative of the likelihood of at least one of: cardiac stress and/or skeletal muscle stress and/or diabetes; and/or a metabolic disorder in the subject.

2. The system of paragraph 1, wherein the threshold level that is indicative of cardiac stress or skeletal muscle stress is at least 30%.

3. The system of paragraph 1, wherein the Fstl1 polypeptide expression level is the expression level of the polypeptide of SEQ ID NO: 1.

4. The system of paragraph 1, wherein the Fstl1 gene expression level is the expression level of the nucleotide of SEQ ID NO: 2 or expression level of Fstl1 mRNA.

5. The system of paragraph 1, wherein the biological sample is selected from the group consisting of: a muscle sample, a tissue sample, a biopsy sample, an ex vivo cultivated sample, a ex vivo cultivated tissue sample, a surgically dissected tissue sample, a blood sample, a plasma sample, a lymph fluid sample, a primary ascite sample, a serum sample, a sputum sample, a saliva sample, a stool sample, a urine sample, a lymph fluid sample, a tear sample, a milk sample.

6. The system of paragraph 1, wherein the biological sample is obtained from a mammalian subject or a human subject.

7. The system of paragraph 1, wherein the cardiac injury is a myocardial infarction.

8. The system of paragraph 1, wherein the Fstl1 polypeptide expression is measured by immuno assay.

9. The system of paragraph 8, wherein the immuno assay is Western blot analysis or ELISA.

10. The system of paragraph 5, wherein the tissue sample is from an organ, skeletal muscle, neuronal tissue.

11. The system of paragraph 5, wherein the tissue sample is a cardiac tissue sample or a skeletal muscle tissue sample.

12. The system of paragraph 1, wherein the subject has experienced one or more symptoms or risk factors for one or more of pressure overload hypertrophy, myocardial infarction, angina pectoris (stable and unstable), cardiomyopathy, myocarditis, congestive heart failure, reinfarction, atherosclerosis, acute coronary syndrome, idiopathic cardiomyopathy, drug-induced cardiomyopathy, alcoholic cardiomyopathy, hypertensive cardiomyopathy, atheromatous disorders of the major blood vessels, metabolic cardiomyopathy, diabetic cardiomyopathy. Ischemic cardiomyopathy, hypertensive cardiomyopathy, dilated cardiomyopathy, hypertrophic cardiomyopathy, arrhythmogenic right ventricular cardiomyopathy, left ventricular hypertrophy, cardiac arrhythmias. restrictive cardiomyopathy, inflammatory cardiomyopathy.

13. The system of paragraph 1, wherein the biological sample is taken from the subject following administration of a cardioprotective agent or a cardioprotective agent which increases the subject's Fstl1.

14. The system of paragraph 1, wherein the a determination module is configured to further determining the level of an additional agent in the biological sample.

15. The system of paragraph 14, wherein the additional agent is a marker of cardiac stress, and/or injury or both.

16. The system of paragraph 1, wherein the skeletal muscle distress is ischemic muscle disease.

17. The system of paragraph 1, wherein the subject is at risk of developing, or has a skeletal muscle ischemia disease.

18. The system of paragraph 17, wherein the skeletal muscle ischemia disease is selected from the group consisting of: muscular dystrophy, muscle atrophy, sarcopenia, inclusion body myocytis.

19. A computer readable medium having computer readable instructions recorded thereon to define software modules including a comparison module and a display module for implementing a method on a computer, said method comprising:

a) comparing with the comparison module the data stored on a storage device with reference data to provide a comparison result, wherein the comparison result is the Fstl1 expression level in the biological above a threshold level relative to a reference Fstl1 expression level that is indicative of cardiac stress or skeletal muscle stress; and

b) displaying a content based in part on the comparison result for the user, wherein the content is a signal indicative of cardiac stress and/or skeletal muscle stress.

20. The computer readable medium of paragraph 19, wherein the threshold level that is indicative of cardiac stress or skeletal muscle stress is 30%.

21. The computer readable medium of paragraph 19, wherein the Fstl1 polypeptide expression level is the expression level of the polypeptide of SEQ ID NO: 1.

22. The computer readable medium of paragraph 19, wherein the Fstl1 gene expression level is the expression level of the nucleotide of SEQ ID NO: 2 or expression level of Fstl1 mRNA.

23. The computer readable medium of paragraph 19, wherein the biological sample is selected from the group consisting of: a muscle sample, a tissue sample, a biopsy sample, an ex vivo cultivated sample, a ex vivo cultivated tissue sample, a surgically dissected tissue.

24. A method of treating a subject at risk for cardiac stress and/or skeletal muscle stress and/or diabetes and/or metabolic dysfunction comprising;

a) determining if the subject is at risk for cardiac stress and/or skeletal muscle stress and/or diabetes and/or metabolic dysfunction by measuring Fstl1 expression level in a biological sample obtained from the subject,

wherein high levels of Fstl1 polypeptide expression or Fstl1 gene expression or Fstl1 mRNA expression in the biological sample indicates the subject is likely to be at risk for cardiac stress and/or skeletal muscle stress, and

wherein low levels of Fstl1 polypeptide expression or Fstl1 gene expression or Fstl1 mRNA expression in the biological sample indicates the subject is likely to be at risk for diabetes and/or metabolic dysfunction,

b). administering to a subject determined to be at risk for cardiac stress and/or skeletal muscle stress a cardioprotective drug or skeletal muscle stress drug, or administering to a subject determined to be at risk for diabetes and/or metabolic dysfunction an appropriate therapy.

25. The method of paragraph 24, wherein the skeletal muscle stress drug is a nutritional program.

### Example

Materials and Methods

Reagents. SYBR GREEN was purchased from Applied Biosystems. The plasmid vector, pcDNA3.1/V5-His was obtained from Invitrogen and adenoviral backbone plasmid pAdEasy-1 was from Qbiogene. Anti-HA antibody was purchased from Roche. Antibodies against Akt, ERK, phospho-Akt, phospho-mTOR and phospho-ERK were purchased from Cell Signaling Technology. Anti-mouse Fstl1 antibody was obtained from R&D Systems. Anti-phospho-FoxO1/3 antibody was from Upstate, antibody against alpha-tubulin was from Oncogene and anti-sarcomeric actin antibody was from Sigma. Dulbecco's modified Eagle's medium (DMEM) was purchased from Invitrogen. U0126 compound was from cell Signaling Technology and LY294002 was from Calbiochem.

Phospho-Akt (Ser473), phospho-eNOS (Ser1177), phospho-p42/44 extracellular signal-regulated kinase (ERK) (Thr202/Tyr204), phospho-GSK-3β (Ser9), and Akt antibodies were purchased from Cell Signaling Technology. eNOS antibody was from Santa Cruz Biotechnology, tubulin antibody was from Oncogene and Fstl1 antibody was obtained from R&D Systems. Anti-human Fstl1 antibody was obtained from Abcam. LY294002 was obtained from Calbiochem, and N -nitro-L-arginine methyl ester (L-NAME) was obtained from Sigma.

RNA isolation, reverse transcriptional PCR, and Quantitative Real-Time PCR. Total RNA from mouse heart samples were prepared by using RNA isolation kit (Qiagen) according to manufacture's protocols with treatment with DNase 1. Using 450ng of total RNA, cDNA were synthesized with random hexamer primer by using ThermoScript RT-PCR Systems (Invitrogen) according to the manufacture's protocols. Quantitative Real-Time PCR (QRT-PCR) was performed on an ABI-Prism 7900 (Applied Biosystems) using SYBR GREEN 1 as a double-stranded DNA-specific dye. Primers were designed to be suitable for a single QRT-PCR thermal profile (95° C. for 10 min, and 50 cycles of 95° C. for 30 s and 60° C. for 1 min). The sequences are as follows: glyceraldehyde-3-phosphate dehydrogenase (GAPDH) forward (F): 5′-TCACCACCATGGAGAAGGC-3′ reverse (R) (SEQ ID NO: 3): 5′-GCTAAGCAGTTGGTGGTGCA-3′ (SEQ ID NO: 4); Fstl1 F: 5′-AACAGCCATCAACATCACCACTTAT-3′ (SEQ ID NO: 5) Fstl R: 5′-TTTCCAGTCAGCGTTCTCATCA-3′ (SEQ ID NO: 6); follistatin F: 5′-CGAGGAGGATGTGAACGACAA-3′ (SEQ ID NO: 7); follistatin R: 5′-GGTCCGCAGTCCACGTTCT-3′ (SEQ ID NO: 8); Fstl3 F: 5′-CAACCCCGGCCAAGAACT-3′ (SEQ ID NO: 9); Fsl3 R: 5′-CTTCCTCCTCTGCTGGTACTTTG-3′ (SEQ ID NO: 10); SPARC F: 5′-ATTGGCGAGTTTGAGAAGGT-3′ (SEQ ID NO: 11); SPARC R: 5′-TTTGCATGGTCCGATGTAGT-3′ (SEQ ID NO: 12); 18S (rat) F: 5′-AATCAAGAACGAAAGTCGGAGG-3′ (SEQ ID NO: 13); 18S(rat) R: 5′-GCGGGTCATGGGAATAACG-3′ (SEQ ID NO: 14); Fstl1 (rat) F: 5′-CTGAAAATTCGTGGAGCAGAA-3′ (SEQ ID NO: 15); Fstl (rat) R: 5′-GTTTCCAGTCAGCGTTATCA-3′ (SEQ ID NO: 16). The expression levels of examined transcripts were compared to that of GAPDH or 18S and normalized to the mean value of controls.

Myocyte cultures. Primary cultures of neonatal rat ventricular myocytes (NRVCs) were prepared and incubated in Dulbecco's modified Eagle's medium (DMEM) supplemented with 7% Fetal Calf Serum (FCS)⁴². Eighteen to 24 hours after preparation, cells were transfected with adenoviral vector at a multiplicity of infection (MOI) of 50 in DMEM without serum for 16 hours and incubated in DMEM without serum for more than 12 hours. Hypoxic conditions were generated using a GasPak system (Becton Dickinson) as described previously. For hypoxia/reoxygenation studies, cells were exposed 12 hr hypoxia followed by 24 hr reoxygenation.

Western blot analysis. Heart tissue was homogenized in lysis buffer (Cell Signaling) containing 1 mM PMSF. The protein concentration was calculated using a BCA protein assay kit (Pierce). Cultured cells were lysed directly in lysis buffer. The cell and tissue lysates or culture media were added to equal volumes of 2× sample buffer (BioRad), and separated by SDS-PAGE. Proteins were transferred onto PVDF membrane (Amersham) and probed with the primary antibody followed by incubation with the HRP-conjugated secondary antibody. ECL plus system (Amersham) was used for detection of the protein signal. To quantify the expression level, the band intensities of Fstl1 were corrected by those of alpha-tubulin by using Image J software.

TUNEL staining. TUNEL staining for cultured cardiac myocytes was performed using the In Situ Cell Death detection kit (Roche) as described previously² with some modifications. In brief, cells are fixed by 4% paraformaldehyde in PBS, permeabilized with 0.1% Triton X-100 and incubated with anti-sarcomeric actinin antibody for 60 min followed by incubation with Cy3-conjugated anti-mouse IgG antibody. Cells were then incubated with TUNEL staining solution for 1 hr according to the manufacture's protocol. DAPI was used for nuclear staining. TUNEL staining for the frozen heart sections was performed as described previously² with some modifications. Cryo-sections (6 μm thickness) were fixed with 4% paraformaldehyde in PBS, permeabilized with 0.1% Triton X-100 and blocked with 6% skim milk. Anti-sarcomeric actin antibody was used for determination of myocytes followed by TUNEL and nuclear staining as described above. TUNEL positive myocytes were counted in randomly selected three fields of the slide and the experiments were repeated three times in duplicates.

Detection of nucleosome fragmentation by ELISA. Nucleosome fragmentation was assessed by ELISA using Cell Death Detection kit (Roche) according to the manufacture's protocol. Cardiac myocytes were seeded in 96-well plates, transfected with adenoviral vector and exposed to H/R. In one assay, 8 wells are used for one group and assays are repeated three times. The extent of nucleosome fragmentation of each group was expressed as relative value in comparison with the value of control group (Ad-βgal transfected cells without H/R).

Mouse ischemia/reperfusion injury and infarction models and evaluation of infarction size. Eight week old male C57BL/6 mice (obtained from Charles River Laboratories) were intravenously injected through the jugular vein with adenovirus (Ad-Fstl1 or Ad-βgal, 1.0×10⁹ p.f.u./mouse). Serum Fstl1 was assayed by Western blot analysis five days after adenovirus delivery. At this time mouse ischemia/reperfusion injury was produced as described previously⁴³. Following anesthetization (pentobarbital 50 mg/kg i.p.) and intubation, a suture was ligated around the proximal left coronary artery (LCA) using a snare occluder. Ischemia followed by reperfusion was accomplished by tightening the snare occluder for 30 min and then loosing it. Myocardial reperfusion was confirmed by changes in ECG as well as by changes in appearance of the heart from pale to bright red. The suture was left in place and chest was closed. For mouse myocardial infarction model, the LCA was occluded by tight ligation without reperfusion. During the surgical procedure, the body temperature was monitored and maintained at 37±1° C. Twenty four hours after reperfusion, the chest was re-opened and the suture was re-tied. Evans Blue was injected at the aortic root to determine the area at risk (AAR). The heart was then excised and incubated with 2,3,5-triphenyltetrazolium chloride (TTC) for 5min at 37° C. to determine the infarction area (IA). Left ventricular area (LVA), AAR and IA were determined by computerized planimetry using Image J (Bethesda, Md., USA).

Mouse transverse aortic constriction model. Mouse transverse aortic constriction model was performed as described previously⁴⁴. Following anesthetization, the thoracic aorta was tied with a 7-0 silk suture along with a 26-gauge blunt needle which was then removed to produce a pressure gradient at the site.

Transgenic mice. The generation of cardiac-specific inducible myrAkt1 TG mice was described previously⁸. Briefly, two transgenic mouse lines (Tet-myrAkt1 and alpha MHC-tTA) were mated to generate double-transgenic mice (DTG), and these mice were maintained with 0.5 mg/ml doxycycline (DOX) in the drinking water resulting in the repression of myrAkt1 gene expression. When DOX is withdrawn from the drinking water, tTA binds to tetO elements and induces myrAkt1 gene expression in the cardiac myocytes. For these studies, the Akt1 transgene was induced for 2 weeks by withdrawing DOX from the drinking water at the age of 12 weeks. Control experiments were performed with MHC-tTA single-transgenic mice that underwent the same Dox treatment protocol as DTG mice.

Microarray analysis. Microarray analysis were performed by Affymetrix GeneChip Mouse Expression Set 430 microarrays and normalized as described in a previous report¹⁸. Gene expression levels were compared before myr-Akt1 induction and those 2 wks after myr-Akt1 induction. Among transcripts that are upregulated by Akt activation, we selected transcripts which have full-length open reading frame cDNAs available in the NCBI web site (http://www.ncbi.nlm.nih.gov/). Amino acid sequences were then examined for signal sequences using Signal IP software (http://www.cbs.dtu.dk/services/SignalP/). Transcripts with signal sequence were analyzed with SOSUI signal beta version software (http://bp.nuap.nagoya-u.ac.jp/sosui/) to predict transcripts with transmembrane domain.

Cloning and in vitro transfection assay of mouse Fstl1. Full-length Fstl1 cDNA was obtained by PCR of RNA isolated from cardiac myocytes and subcloned into pcDNA3.1/V5-His that express mouse Fstl1 as a fusion to the V5 epitope at the C-terminus. To test the secretion of the gene product the plasmid vector pcDNA3.1/V5-His expressing Fstl1 was transfected into HEK293 cells using Lipofectamin2000 (Invitrogen). After cells were incubated with serum free media for 24 hours, the cell lysate and media were collected. Cells were mock-transfected (no plasmid) as a negative control. The collected media was concentrated approximately ten fold by using Microcon (Millipore). Cell lysates and media were separated by SDS-PAGE and Fstl1 fused with V5 was detected by Western blot analysis using anti-V5 antibody (Invitrogen).

Construction of adenoviral vector expressing mouse Fstl1. Full-length mouse Fstl1 cDNA was subcloned into an adenovirus shuttle vector. After linearization, shuttle vector was cotransformed into E. coli with the adenoviral backbone plasmid pAdEasy-1. The resultant recombinant adenoviral DNA with Fstl1 cDNA was transfected into HEK 293 cells to produce the recombinant adenoviral vector. For some experiments, an adenoviral vector expressing β-galactosidase (Ad-βgal) was used as a control. Adenoviral vectors were purified by CsCl ultracentrifugation.

RNA interference in ventricular myocytes. The rat Fstl1 small interfering RNA (siRNA) Smart Pool was purchased from Dharmacon Inc and the second siRNA targeting Fstl1 and unrelated siRNA were from Qiagen. The sequences of siRNAs used in this study were: Fstl1 (Dharmacon) mixture of four siRNAs, 5′-UGCAAAUACUUACGGACUUUU-3′ (SEQ ID NO: 17), 5′-CAGAUGGAGCUGAGACCGAUU-3′ (SEQ ID NO: 17), 5′-CCGUCAACAUCACCGCUUAUU-3′ (SEQ ID NO: 18), and 5′-UGGCUAACGGGCAGAGCGAUU-3′ (SEQ ID NO: 19)and Fstl1-b (Qiagen): 5′-r(GCAUCUUGAGAUUUAAUCA)dTdT-3′ (SEQ ID NO: 20). Neonatal rat ventricular myocytes (NRVMs) were transfected with siRNA by Lipofectamine 2000 according to the manufacture's protocol. Forty eight hours after transfection, the protein or mRNA were extracted for western blot analysis or QRT-PCR or cells were exposed to hypoxia/reoxygenation.

Muscle-specific Akt1 Transgenic Mice. The generation of skeletal muscle-specific inducible myrAkt1 TG mice was described previously (9). Briefly, tetracycline-responsive element constitutively-active myrAkt1 (TRE-myrAkt1) TG mouse line was crossed with 1256 [3Emut] MCK-rtTA lines that express reverse tetracycline transactivator (rtTA) from 1256 [3Emut] MCK promoter to generate double-transgenic mice (DTG). To activate Akt1 transgene, DTG were administered doxycycline (DOX, 0.5 mg/ml) in their drinking water. Single 1256 [3Emut] MCK-rtTA TG littermates were used as controls and treated with DOX in the same way as DTG mice.

Mouse Model of Angiogenesis. Male wild-type and eNOS-deficient (eNOS-KO) mice (Jackson Laboratory) in a C57/BL6 background were used for this study. Study protocols were approved by the Institutional Animal Care and Use Committee in Boston University. Mice, at the ages of 10 weeks, were subjected to unilateral hindlimb surgery under anesthesia with sodium pentobarbital (50 mg/kg intraperitoneally). In this model, the entire left femoral artery and vein were excised surgically (16,17). In some experiments, the 2x10⁸ plaque-forming units (pfu) of adenoviral constructs encoding Fstl1 (Ad-Fstl1) or expressing β-galactosidase (Ad-βgal), as a control, were injected into five different sites of adductor muscle in the ischemic limb 3 days prior to the ischemic hindlimb as previously described (16,17). Hindlimb blood flow was measured using a laser Doppler blood flow (LDBF) analyzer (Moor LDI; Moor Instruments) immediately before surgery and on postoperative days 3, 7, and 14. Hindlimb blood flow was expressed as the ratio of left (ischemic) to right (nonischemic) LDBF. Following sacrifice, capillary density within gastrocnemius or thigh adductor muscle was quantified by histological analysis (16,17). Muscle samples were imbedded in OCT compound (Miles, Elkhart, Ind., USA) and snap-frozen in liquid nitrogen. Tissue slices (5 μm in thickness) were stained with anti-CD31 (PECAM-1: Becton Dickinson) antibodies. Fifteen randomly chosen microscopic fields from three different sections in each tissue block were examined for the presence of CD31-positive capillary endothelial cells. Capillary density was expressed as the number of CD31-positive cells per muscle fiber.

Cell Culture, Adenoviral Infection and Western Blot Analysis. Human umbilical vein endothelium cells (HUVECs) were cultured in endothelial cell growth medium-2 (EGM-2, Lonza) (18). HUVECs were infected with adenoviral constructs encoding mouse Fstl1 (Ad-Fstl1) (15), or Ad-β-gal at a multiplicity of infection (MOI) of 10 for 8 h and placed in endothelial cell basal medium-2 (EBM-2, Lonza) without serum for indicated lengths of time. In some experiments, HUVECs were treated with LY294002 (10 μM), L-NAME (1 mg/ml) or vehicle along with transduction with Ad-Fstl1 or Ad-βgal. In some experiments, HUVECs were infected with adenoviral constructs encoding dominant-negative Akt1 (Ad-dnAkt)(18,19) or Ad-βgal at a MOI of 10 together with Ad-Fstl1 or Ad-β-gal. C2C12 mouse myoblasts (American Type Culture Collection) were maintained in growth medium (DMEM supplemented with 20% FBS) and shifted to differentiation medium (DMEM supplemented with 2% heat-inactivated horse serum) for 4 days to induce differentiation (16). C2C12 myocytes were infected with Ad-Fstl1, or Ad-β-gal at a MOI of 250 for 16 h followed by incubation with serum free DMEM for 24 h. Cell and tissue lysates or culture media were resolved by SDS-PAGE. The membranes were immunoblotted with the indicated antibodies at a 1:1000 dilution followed by the secondary antibody conjugated with horseradish peroxidase (HRP) at a 1:5000 dilution. ECL Western Blotting Detection kit (Amersham) was used for detection.

Determination of Fstl1 mRNA. Total RNA was prepared by Qiagen using the manufacturer's suggested protocol, and cDNA was produced using ThermoScript RT-PCR Systems (Invitrogen). Quantitative Real-time PCR (QRT-PCR) was performed on iCycler iQ Real-Time PCR Detection System (BIO-RAD) using SYBR Green I as a double stranded DNA specific dye as described previously (16). Primers were: 5′-AACAGCCATCAACATCACCACTTAT-3′ (SEQ ID NO: 21) and 5′-TTTCCAGTCAGCGTTCTCATCA-3′ (SEQ ID NO: 22) for mouse Fstl1, 5′-TCACCACCATGGAGAAGGC-3′ (SEQ ID NO: 23) and 5′-GCTAAGCAGTTGGTGGTGCA-3′ (SEQ ID NO: 24) for mouse GAPDH.

Migration Assay. Migratory activity was measured using a modified Boyden chamber assay (18). Serum-deprived cells were trypsinized and resuspended in EBM-2 in the absence of serum. Cell suspensions (250 μl, 2.0×10⁴ cells/well) were added to the transwell insert (8.0 μm pore size, Becton Dickinson). After 18 h, migrated cells on the lower surface of the membrane were fixed, stained with Giemsa stain solution and eight random microscopic fields per well were quantified.

Differentiation Assay. The formation of network structures by HUVECs on growth factor-reduced Matrigel (Becton Dickinson) was performed as previously described (18). Twenty-four-well culture plates were coated with Matrigel according to the manufacturer's instructions. HUVECs were seeded on coated plates at 5×10⁴ cells/well in serum-free EBM-2 and incubated at 37° C. for 18 h. Network formation was observe using an inverted phase contrast microscope (Nikon). Images were captured with a video graphic system (DEI-750 CE Digital Output Camera, Optronics). The degree of differentiation into vascular-like structures was quantified by measuring the network areas in three randomly chosen fields from each well using Image J program.

Analysis of Apoptotic Activity. Cells were transduced with adenoviral constructs for 8 h followed by incubation with serum-free EBM-2 for 48 h. Nucleosome fragmentation was assessed by ELISA using Cell Death Detection kit (Roche). Cell viability was also measured by MTS reagent using the CellTiter 96 AQᵤₑₒᵤₛ kit (Promega) (20). TUNEL staining was performed using the In Situ Cell Death detection kit (Roche) (21). TUNEL positive cells were counted in five randomly selected microscopic fields. Each experiment was repeated four times.

Statistical Analysis. All data are expressed as means±SD or SEM as indicated in the figure legends. Differences were analyzed by Student's unpaired t test or ANOVA for multiple comparisons. Group differences were analyzed by two-tailed Student's t test or ANOVA. To compare multiple groups, Mann-Whitney U-test with Bonferroni correction was used. A value of P<0.05 was considered as statistically significant.

### Example 1

Follistatin-Like 1 is an Akt-Regulated Cardioprotective Factor that is Secreted by the Heart

Fstl1 Upregulation by Akt Activation in the Heart.

Microarray gene expression analysis was compared between control mouse hearts and two weeks after myocardial induction of myristoylated Akt1 by withdrawing Dox from the drinking water of DTG mice¹⁸. Transcripts upregulated by 2 wks Akt activation with full-length open reading frame cDNAs were selected. Amino acid sequences were then examined for signal sequences using Signal IP software. Transcripts with signal sequence were further analyzed with SOSUI signal beta version software to exclude transcripts predicted to encode proteins with transmembrane domains. Akt-regulated transcripts were identified that contained a predicted signal sequence but lacked a transmembrane domain. Of this subset of proteins, Follistatin-like 1 (Fstl1) was selected for further analysis because it was upregulated 2.7-fold. To confirm the changes in Fstl1 mRNA level, QRT-PCR was performed using specific primer sets for Fstl1 and normalizing the signal to that of GAPDH (Table 1). Fstl1 was upregulated 2.0-fold by Akt activation in the heart (P<0.05). In contrast, transcripts encoding other proteins containing the follistatin domain, including follistatin, follistatin like-3 and SPARC, were not regulated by activation of the Akt transgene in heart analyzed as assessed by microarray and QRT-PCR analyses (Table 1). Fstl1 transcript and protein expression in mouse heart was not affected by the presence or absence of Dox in the drinking water of control mice.

TABLE 1
Upregulation of Fstl1 transcript by cardiac
myocyte-specific overexpression of Akt.

| Gene name | Fold increase (v.s. control) |
|---|---|
| Follistatin-like 1 | 2.04 ± 0.10* |
| Follistatin | 0.36 ± 0.13 |
| Follistatin-like 3 | 0.68 ± 0.08 |
| SPARC | 0.73 ± 0.07 |
| Akt1 | 7.19 ± 0.83* |

Each value is expressed as mean ± SE. n = 3 for each group. SPARC indicates secreted acidic cysteine rich glycoprotein.
*P < 0.05 compared to control.

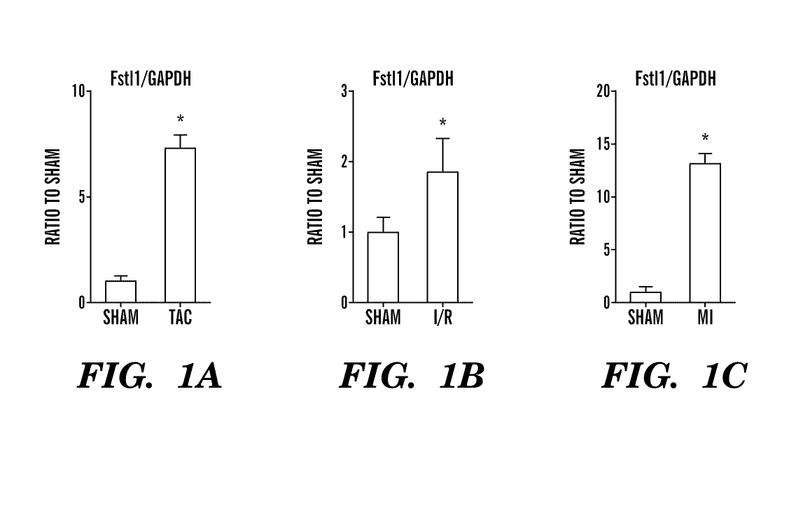

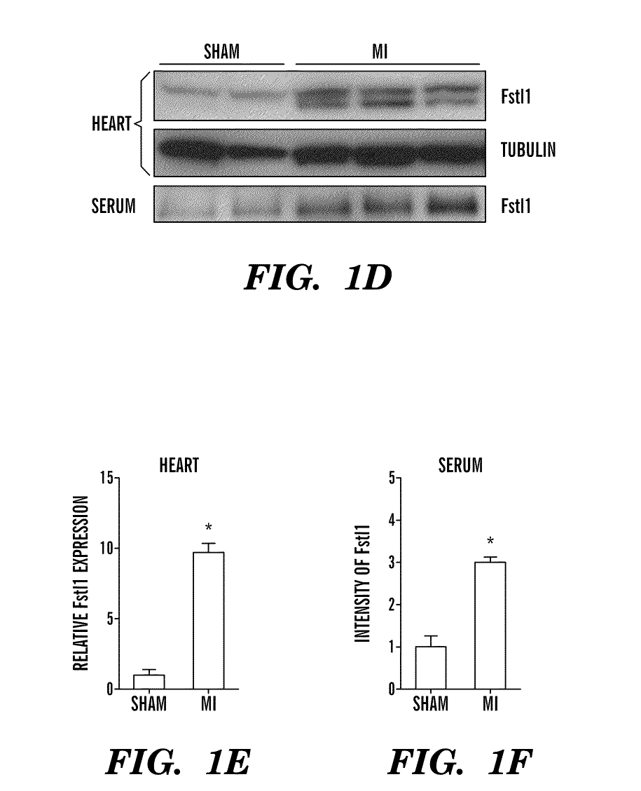

To better understand the regulation of Fstl1 in cardiac cells, transcript levels of this factor were measured by RT-PCR in hearts that were subjected to various injuries. Fstl1 transcript was upregulated approximately 7-fold at the 7 day time point following transverse aortic constriction that induces pressure overload hypertrophy (FIG. 1A). Fstl1 transcript was upregulated approximately 2-fold in the myocardial area at risk 1 day following ischemia-reperfusion injury (FIG. 1B) and approximately 13-fold in hearts 3 days following permanent LAD ligation (FIG. 1C). Fstl1 protein levels were analyzed by Western immunoblot analysis following myocardial infarction because the changes in transcript level were robust in this model. LAD ligation led to a substantial increase in Fstl1 protein in the heart (FIG. 1D). Fstl1 immunoreactive material appeared as a doublet with electrophoretic mobilities indicative of 37 kDa and a 46 kDa proteins. Quantitation of the 37 kDa band indicated a 10-fold upregulation of the Fstl1 protein (FIG. 1E). Fstl1 protein could also be detected in the serum following Western immunoblot with an electrophoretic mobility that corresponded to that of a 46 kDa protein. Following myocardial infarction, the 46 kDa form of Fstl1 increased 3-fold in serum (FIG. 1F). Thus both Fstl1 protein and transcript are induced by a variety of stresses in the heart.

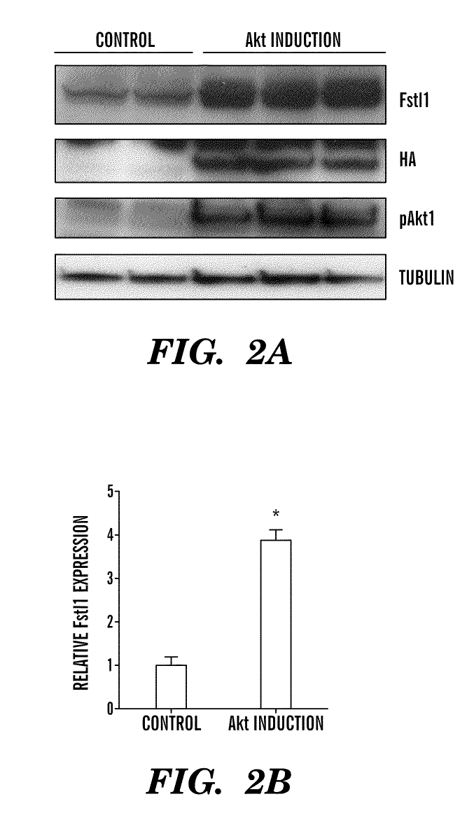

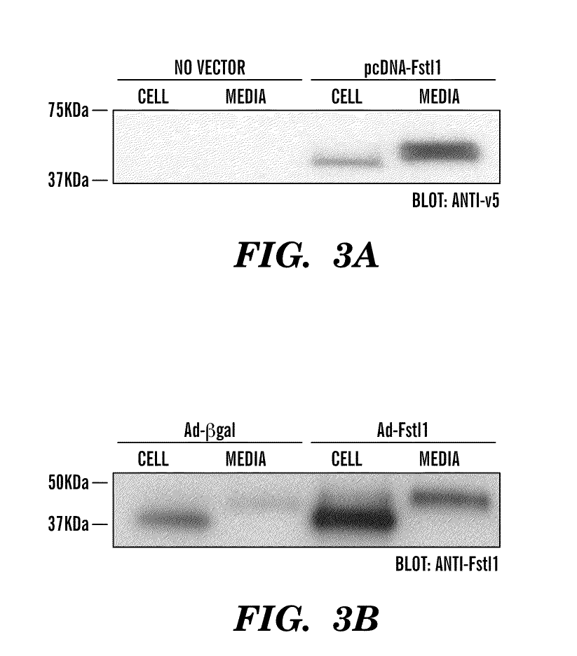

Western blot analysis was performed to assess the Akt-mediated change in Fstl1 protein expression in the heart. FIG. 2A shows Western immunoblots from two control hearts and three myrAkt1 transgenic hearts following two weeks of induction. The expression of Fstl1 protein, both the high and low molecular weight forms, was increased in heart by Akt transgene induction (FIG. 2A). The hemagglutinin (HA)-tagged Akt transgene product was detected by anti-HA antibody and Akt activation was confirmed by phospho-specific anti-Akt antibody (FIG. 2A). After normalization to the alpha-tubulin signal, Fstl1 protein expression was induced approximately 4-fold by Akt activation in the heart (FIG. 2B).

Fstl1 is a Secreted Protein

Plasmid and adenovirus vectors expressing Fstl1 were constructed. Using cDNA mouse heart, Fstl1 was subcloned into the pcDNA3.1/V5-His expression vector. The vector was transfected into HEK293 cells, and cells were incubated with serum free media for 24 hours. As shown in FIG. 3A, the tagged Fstl1 protein was detected both in the cell pellet lysate as well as in the media indicating that it is secreted from HEK293 cells. The apparent molecular weight of Fstl1, based upon electrophoretic mobility, was greater in the media than in the cell pellet lysate.

An adenoviral expression vector expressing non-tagged Fstl1 was also constructed and tested. Neonatal rat ventricular cardiomyocytes (NRVMs) were transduced with Ad-Fstl1 or Ad-βgal, as a control, and Fstl1 protein was detected both in the cell pellet and the media (FIG. 3B). In addition, lower intensity Fstl1 signals, corresponding to endogenous Fstl1 expression could be detected in the lysate and media of cells transduced with Ad-βgal (FIG. 3B). As with the plasmid-encoded Fst1 produced by HEK293 cells, the protein detected in the NRVM media exhibited a reduced electrophoretic mobility indicating that the secreted protein is post-translationally modified. The predicted molecular weight of the protein in the NRVM pellet corresponded to 37 kDa, in good agreement with the predicted molecular weight of 34.4 kDa for the 306 amino acid Fstl1 protein.

Fstl1 Prevents NRVMs from Hypoxia/Rreoxygenation Induced Apoptosis through Activation of Akt and ERK Signaling

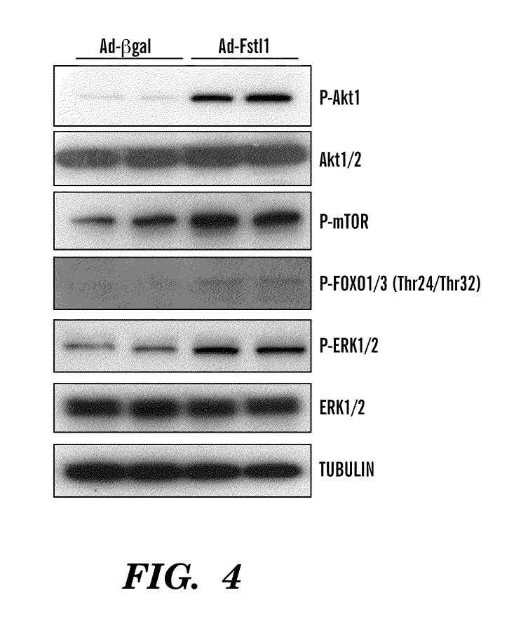

There are no reports of Fstl1-induced changes in intracellular signaling. Thus, NRVMs in serum-free media were transduced with Ad-Fstl1 and Western immunoblot analysis of signaling proteins was performed. Adenovirus-mediated overexpression of Fstl1 in NRVMs led to a marked increase in the activating phosphorylation of Akt at Ser473 (FIG. 4). Consistent with an increase in Akt signaling, increases in the phosphorylation of mTOR and FOXO proteins, downstream targets of Akt, were also observed. Transduction with Ad-Fstl1 also led to an increase in ERK phosphorylation in NRVMs.

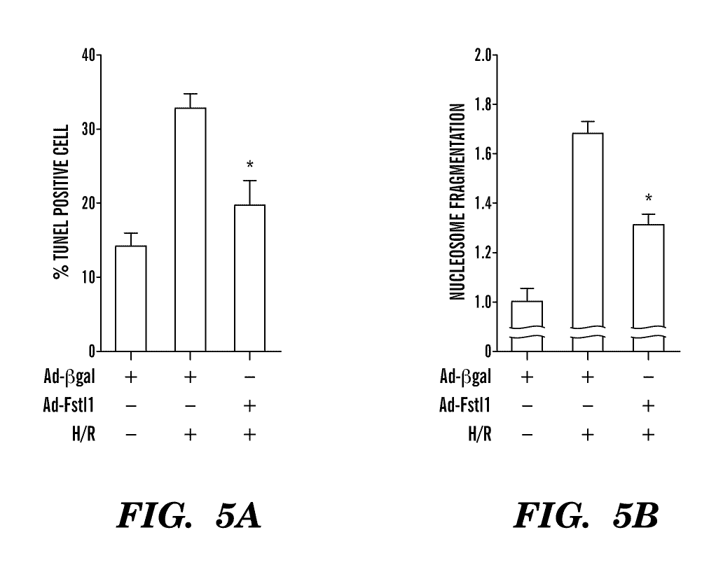

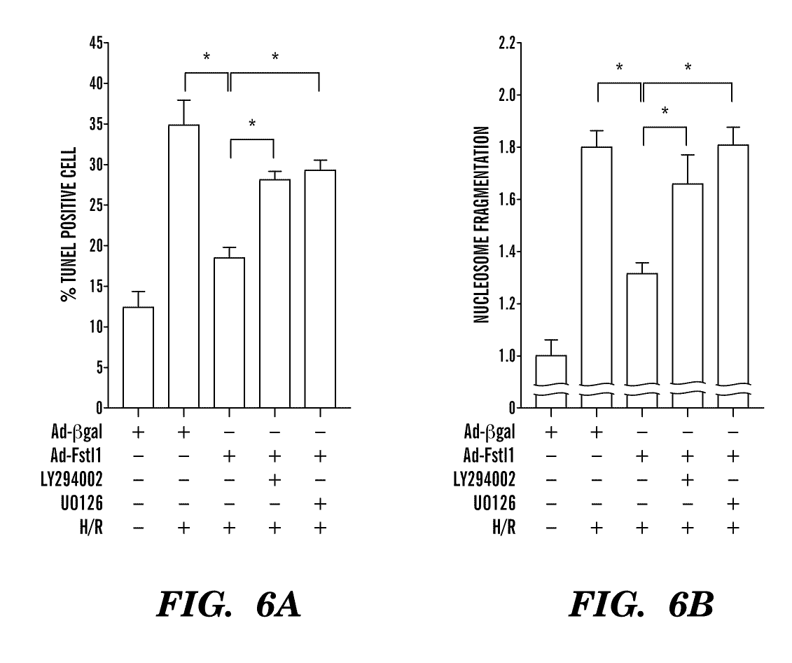

Both Akt and ERK signaling promote cardiomyocyte survival^{2,21}. Thus, we examined the effect of Fstl1 expression on apoptosis after hypoxia/reoxygenation (H/R) in NRVMs. Cells were transduced with Fstl1-expressing and control adenoviral vectors, and then exposed to media without serum. Parallel cultures were maintained in serum free media and exposed to normoxia or 12 hours of hypoxia followed by 24 hours of reoxygenation (H/R). TUNEL staining was performed to evaluate the effect of Fstl1 on cardiac myocyte apoptosis. As shown in FIG. 5A, treatment with Ad-Fstl1 reduced the frequency of TUNEL-positive cells under conditions of H/R stress. Quantification of TUNEL-positive myocytes revealed that Ad-Fstl1 significantly reduced TUNEL positive cell number after H/R compared to Ad-βgal transfected cells (P=0.008). To corroborate the effects of Fstl1 on apoptosis, nucleosome fragmentation was assessed by ELISA. As shown in the FIG. 5C, H/R induced nucleosome fragmentation was significantly suppressed by Ad-Fstl1 (P=0.004).

To examine the functional significance of Akt and ERK in Fstl1-mediated cytoprotection, NRVMs were pre-treated with specific inhibitors and subjected to H/R. Cells were exposed to LY294002 (10 μmol/L), a PI3K inhibitor, or with U0126 (10 μmol/L), a MEK 1/2 inhibitor, 3 hours prior to H/R stress. FIG. 6A shows representative fluorescent photographs of TUNEL staining for each experimental group. As shown in FIG. 6B, transduction with Ad-Fstl1 reduced TUNEL positive cells after exposure to H/R compared with cells treated with Ad-βgal, but the protective action of Ad-Fstl1 was partially attenuated by pre-incubation with LY29402 or U0126 (P<0.05). In agreement with these findings, the inhibitory effect of Fstl1 on nucleosome fragmentation was significantly attenuated by treatment with each of inhibitor (FIG. 6C).

Fstl1 Functions as an Endogenous Cardiac Myocyte Survival Factor

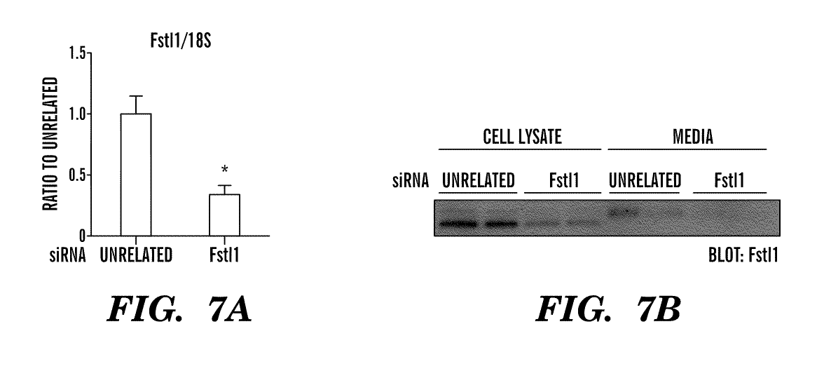

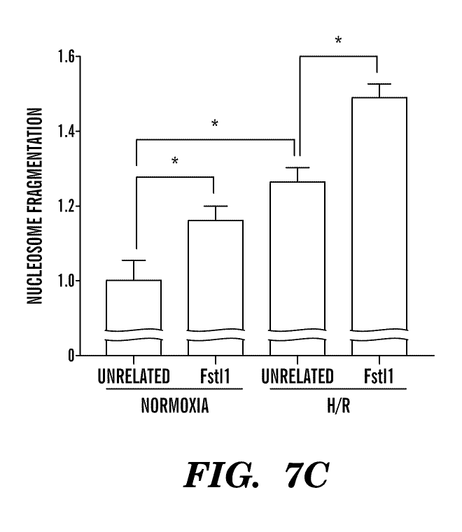

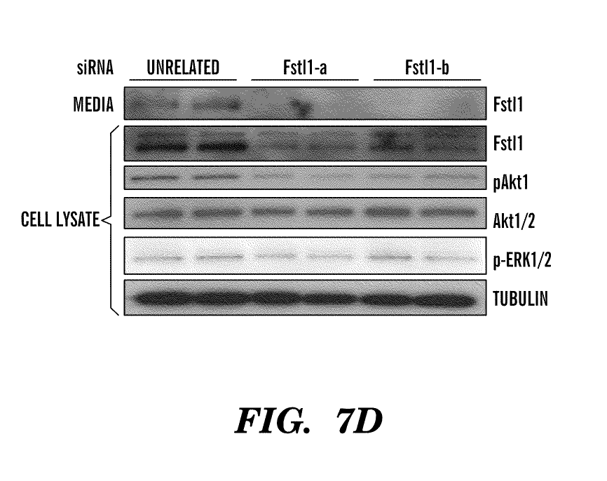

To assess the role of endogenous Fstl1, NRVMs were transfected with siRNA targeting Fstl1. Forty eight hours after transfection, both mRNA and protein levels of Fstl1 were significantly attenuated as shown by QRT-PCR and Western blot analysis (FIGS. 7A and 7B). Fstl1 protein expression level was decreased by 85% in cell lysate and by 90% in culture media by siRNA treatment (P<0.001). At this time point nucleosome fragmentation was analyzed in serum-deprived cells. As shown in the FIG. 7C, knock-down of Fstl1 resulted in increased apoptosis in normoxic cells as well as cells subjected to H/R stress, suggesting that endogenous Fstl1 plays a role in maintaining cardiomyocyte viability. Fstl1 knock-down resulted in reduced phosphorylation of Akt at baseline, but had no effect on baseline ERK signaling (FIG. 7D). These Western immunoblot observations were corroborated using second source of siRNA targeting Fstl1 (FIG. 7D).

Systemic Administration of Fstl1 Protects the Myocardium from Ischemia/Reperfusion Injury

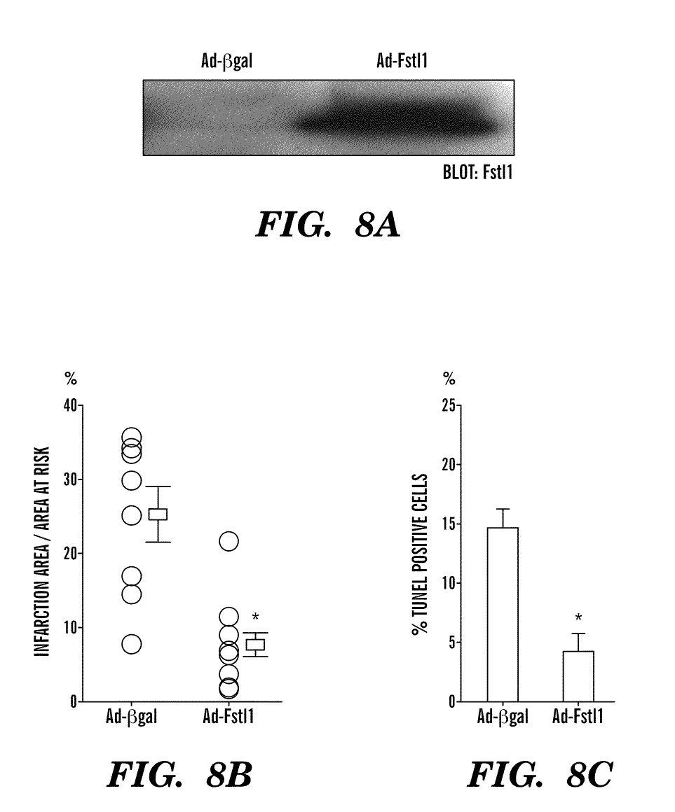

To assess in vivo actions of Fstl1 on the heart, mice were injected intravenously with Ad-Fstl1 or Ad-βgal (1.0×10⁹ p.f.u./mouse) prior to myocardial ischemia-reperfusion injury. This mode of adenovirus delivery leads to transduction of the liver but not heart²², thereby allowing the assessment of the cardioprotective properties of the secreted form of Fstl1. Five days after the delivery of Ad-Fstl1, serum Fstl1 levels were markedly elevated (FIG. 8A). At this time point, mice were exposed to 30 minutes of myocardial ischemia followed by 24 hours of reperfusion. FIG. 8B shows representative heart sections following staining for TTC. Prior treatment with Ad-Fstl1 resulted in a 66.0% decrease in myocardial infarct size compared with mice treated with Ad-βgal (FIG. 8C). To investigate the extent of apoptosis in the area at risk, TUNEL staining on the different experimental groups were performed. Representative fluorescent photographs of each group are shown in FIG. 8D. Quantitative analysis indicated significantly fewer TUNEL-positive myocytes in the myocardium of mice that were treated with Ad-Fstl1 (FIG. 8E, P<0.001).

In this study, the inventors have discovered that Fstl1 transcript and protein are upregulated in the myocardium after Akt1 transgene activation. Our studies have also revealed that Fstl1 is a secreted protein, and that its overexpression will promote Akt and ERK1/2 signaling in cardiac myocytes and protect cardiac myocytes from hypoxia/reoxygenation-induced apoptosis through activation of these pathways. Conversely, siRNA-mediated knockdown of Fstl1 led to an increase in the frequency of stress-induced apoptosis, and this was associated with a reduction in basal Akt phosphorylation. Fstl1 expression was also upregulated in the heart following pathological stimuli including pressure overload, ischemia-reperfusion and permanent LAD ligation. Finally, the systemic delivery of Fstl1 protected myocardium from ischemia-reperfusion injury which was accompanied with a reduction in apoptosis.

A number of lines of evidence suggest that Fstl1 is secreted from cardiac myocytes. Cultured cardiac myocyte cultures display immuno-detectable Fstl1 protein in the cell media and this signal is increased when cultures are transduced with the Fstl1 gene. The secreted form of Fstl1 has a larger molecular weight, presumably the result of protein glycosylation²³. Furthermore, an appreciable increase in the high molecular weight form of Fstl1 could be detected in mouse serum following myocardial infarction. Fstl1 has also been detected in human serum²³, indicating that Fstl1 could have diagnostic utility. The heart secretes factors that maintain its performance and produce systemic actions. For example, atrial natriuretic peptide and brain natriuretic peptide are well-known cardiac hormones produced in the heart that serve as therapeutic or diagnostic agents^{24,25}. Therefore, newly identified factors secreted by the myocardium, such as Fstl1, are also be candidates for clinical applications.

Recent reports have proposed that Akt-activated myocardium produce factors that are important in maintenance of cardiac homeostasis. The inventors have previously demonstrated that VEGF and angiopoietin-2 are upregulated in Akt-activated myocardium⁸, and VEGF secretion was found to be necessary for compensatory hypertrophy^{8,26}. Others have demonstrated that mesenchymal stem cells expressing Akt improve cardiac performance when implanted into myocardium after myocardial infarction, and that factors secreted from the Akt-activated mesenchymal stem cells confer the cardioprotective benefits¹⁷. Herein the inventors demonstrate that that Akt-activated cells are a source of factors that are important in cardiac repair and regeneration.

The follistatin family of proteins are generally believed to function by binding to and modifying the function of members of the TGF-β superfamily²⁷. Follistatin binds to Growth and Differentiation Factor 8 (GDF8), also referred to as myostatin, as well as activin A and B, and various Bone Morphogenic Proteins (BMP) including BMP-2, 4 and 7. Similarly Fstl3 binds to a subset of these TGF-β superfamily members including myostatin. Myostatin (GDF8) is of interest because it is expressed by the heart, and it is reported to have anti-hypertrophic and anti-proliferative actions on cultured cardiac myocytes^{28,29}. However, it is controversial whether myostatin controls heart size in vivo^{30,31}. Also of interest, GDF15 is upregulated in the heart by pressure overload or ischemic injury, and it exhibits cardioprotective functions^{32,33}. It is also reported that serum levels of GDF15 serve as an independent predictor of adverse events in patients with chronic heart failure and acute coronary syndrome^{34,35}. Finally, BMP-2 has been reported to act on cardiac myocytes to inhibit apoptosis via the SMAD pathway³⁶.

In contrast to the considerations discussed above, it is not clear whether Fstl1 functions by binding to TGF-β superfamily proteins in a manner similar to follistatin or Fstl3³⁷. In this regard, follistatin and Fstl3 display a 29% amino acid sequence identity in mice, but Fstl1 shares only 7% homology with follistatin and 6% homology with Fstl3. Without beingn bound by theory, we do not favor the hypothesis that Fstl1 acts indirectly on cardiac myocytes through its ability to bind to TGF-β superfamily protein members. In cell culture experiments, we found that Fstl1 expression promoted the activation of signaling pathways and promoted cell viability in response to hypoxia-reoxygenation. Because these cell culture experiments were performed in serum-free media, it is unlikely that Fstl1 alters cellular responses in cardiac myocytes solely through its ability to modulate the function of the binding partner. Another protein with higher homology to Fstl1 than follistatin or Fstl3 is SPARC which exhibits a 14% sequence identity. Thus, Fstl1 may be functionally more similar to SPARC than to follistatin and Fstl3, and the structural similarity with SPARC suggests that Fstl1 may function to modulate intracellular signaling by controlling cell interactions with extracellular matrix.

Adenovirus-mediated Fstl1 overexpression led to the upregulation of Akt and ERK signaling in cardiac myocytes and improved cardiac myocyte survival. Furthermore, conditioned media from myocytes transduced with Ad-Fstl1 activated Akt signaling and protected against H/R stress-induced apoptosis in cultures of NRVMs (Y.O. unpublished results). Since Akt and ERK are involved in cellular survival^{2,21,38}, it is likely that the cardioprotective actions of exogenous Fstl1 result from the upregulation of these signaling systems. Consistent with this hypothesis, treatment with inhibitors of PI3K or ERK diminished the anti-apoptotic activity of exogenous Fstl1. Furthermore, knock-down of endogenous Fstl1 led to a reduction in Akt phosphorylation and an increase in the frequency of myocyte apoptosis. Considering that Fstl1 is upregulated in the Akt-activated heart, it appears that Fstl1 could act as a positive feedback loop to promote myocyte survival, or it could act to promote the survival of neighboring myocytes. In this regard, it has been reported that myocyte survival is also modulated by Pim-1, heme oxygenase-1 and hypoxia-inducible-factor 1a which function within the Akt signaling network³⁹⁻⁴¹.

In conclusion, these data show that Fstl1 is secreted by cardiac myocytes and that this factor is upregulated in the heart by an activation of Akt signaling or by injury. Overexpression of Fstl1 is cardioprotective, whereas ablation of Fstl1 leads to an increase in cardiac myocyte apoptosis. We teach that Fstl1 is a cardioprotective factor that is secreted by the heart.

### References for Example 1

The references below refer to references cited in Example 1 and all references cited as “Superscript” (i.e. ¹) in Example 1 and the “material and methods”. All references described herein are incorporated herein by reference in their entirety.

1. Shiojima I, Walsh K. Regulation of cardiac growth and coronary angiogenesis by the Akt/PKB signaling pathway. Genes Dev. 2006;20:3347-3365.

2. Fujio Y, Nguyen, T., Wencker, D., Kitsis, R. N., and Walsh, K. Akt promotes survival of cardiomyocytes in vitro and protects against ischemia-reperfusion injury in mouse heart. Circulation. 2000;101:660-667.

3. Miao W, Luo, Z., Kitsis, R. N., and Walsh, K. Intracoronary, adenovirus-mediated Akt gene transfer in heart limits infarct size following ischemia-reperfusion injury in vivo. J. Mol. Cell. Cardiol. 2000;32:2397-2402.

4. Taniyama Y, Walsh K. Elevated myocardial Akt signaling ameliorates doxorubicin-induced congestive heart failure and promotes heart growth. J Mol. Cell Cardiol. 2002;34:1241 -1247.

5. Condorelli G, Drusco A, Stassi G, Bellacosa A, Roncarati R, laccarino G, Russo M A, Gu Y, Dalton N, Chung C, Latronico M V, Napoli C, Sadoshima J, Croce C M, Ross J, Jr. Akt induces enhanced myocardial contractility and cell size in vivo in transgenic mice. Proc. Natl. Acad. Sci. USA. 2002;99:12333-12338.

6. Matsui T, Li L, Wu J C, Cook S A, Nagoshi T, Picard M H, Liao R, Rosenzweig A. Phenotypic spectrum caused by transgenic overexpression of activated Akt in the heart. J. Biol. Chem. 2002;277:22896-22901.

7. Taniyama Y, Ito M, Sato K, Kuester C, Veit K, Tremp G, Liao R, Colucci W S, Ivashchenko Y, Walsh K, Shiojima I. Akt3 overexpression in the heart results in progression from adaptive to maladaptive hypertrophy. J. Mol. Cell. Cardiol. 2005;38:375-385.

8. Shiojima I, Sato, K., Izumiya, Y., Schiekofer, S., Ito, M., Liao, R., Colucci, W. S., and Walsh, K. Disruption of coordinated cardiac hypertrophy and angiogenesis contributes to the transition to heart failure. J. Clin. Invest. 2005;115:2108-2118.

9. Shiraishi I, Melendez J, Ahn Y, Skavdahl M, Murphy E, Welch S, Schaefer E, Walsh K, Rosenzweig A, Torella D, Nurzynska D, Kajstura J, Leri A, Anversa P, Sussman M A. Nuclear targeting of Akt enhances kinase activity and survival of cardiomyocytes. Circ. Res. 2004;94:884-891.

10. Rota M, Boni A, Urbanek K, Padin-Iruegas M E, Kajstura T J, Fiore G, Kubo H, Sonnenblick E H, Musso E, Houser S R, Leri A, Sussman M A, Anversa P. Nuclear targeting of Akt enhances ventricular function and myocyte contractility. Circ. Res. 2005;97:1332-1341.

11. de Bold A J, Borenstein, H. B., Veress, A. T. and Sonnenberg, H. A rapid and potent natriuretic response to intravenous injection of atrial myocardial extract in rats. Life Sci. 1981;28:89-94.

12. Flynn T G, de Bold, M. L. and de Bold, A. J. The amino acid sequence of an atrial peptide with potent diuretic and natriuretic properties. Biochem. Biophys. Res. Commun. 1983;117:859-865.

13. Maekawa K, Sudoh, T., Furusawa, M., Minamino, N., Kangawa, K., Ohkubo, H., Nakanishi, S. and Matsuo, H. Cloning and sequence analysis of cDNA encoding a precursor for porcine brain natriuretic peptide. Biochem. Biophys. Res. Commun. 1988;157:410-416.

14. Funamoto M, Hishinuma, S., Fujio, Y., Matsuda, Y., Oh, H., Negoro, S., Tone, E., Kishimoto, T. and Yamauchi-Takihara, K. Isolation and characterization of the murine cardiotrophin-1 gene: expression and norepinephrine-induced transcriptional activation. J. Mol. Cell. Cardiol. 2000;32:1275-1284.

15. Tsuruda T, Kato, J., Kitamura, K., Kuwasako, K., Imamura, T., Koiwaya, Y., Tsuji, T., Kangawa, K. and Eto, T. Adrenomedullin: a possible autocrine or paracrine inhibitor of hypertrophy of cardiomyocytes. Hypertension. 1998;31:505-510.

16. Frost R J, Engelhardt S. A secretion trap screen in yeast identifies protease inhibitor 16 as a novel antihypertrophic protein secreted from the heart. Circulation. 2007;116:1768-75.

17. Gnecchi M, He, H., Liang, O. D., Melo, L. G., Morello, F., Mu, H., Noiseux, N., Zhang, L., Pratt, R. E., Ingwall, J. S., and Dzau, V. J. Paracrine action accounts for marked protection of ischemic heart by Akt-modified mesenchymal stem cells. Nat. Med. 2005;11:367-368.

18. Schiekofer S, Shiojima, I., Sato, K., Galasso, G., Oshima, Y., and Walsh, K. Microarray analysis of Akt1 activation in transgenic mouse hearts reveals transcript expression profiles associated with compensatory hypertrophy and failure. Physiol. Genomics. 2006;27:156-170.

19. Sumitomo K, Kurisaki A, Yamakawa N, Tsuchida K, Shimizu E, Sone S, Sugino H. Expression of a TGF-beta1 inducible gene, TSC-36, causes growth inhibition in human lung cancer cell lines. Cancer Lett. 2000; 155:37-46.

20. Johnston I M, Spence, H. J., Winnie, J. N., McGarry, L., Vass, J. K., Meagher, L., Stapleton, G., and Ozanne, B. W. Regulation of a multigenic invasion programme by the transcription factor, AP-1: re-expression of a down-regulated gene, TSC-36, inhibits invasion. Oncogene. 2000;19:5348-5358.

21. Yue T L, Wang, C., Gu, J. L., Ma, X. L., Kumar, S., Lee, J. C., Feuerstein, G. Z., Thomas, H., Maleeff, B. and Ohlstein, E. H. Inhibition of extracellular signal-regulated kinase enhances Ischemia/Reoxygenation-induced apoptosis in cultured cardiac myocytes and exaggerates reperfusion injury in isolated perfused heart. Circ. Res. 2000;86:692-699.

22. Shibata R, Sato K, Kumada M, Izumiya Y, Sonoda M, Kihara S, Ouchi N, Walsh K. Adiponectin accumulates in myocardial tissue that has been damaged by ischemia-reperfusion injury via leakage from the vascular compartment. Cardiovasc. Res. 2007;74:471-479.

23. Liu T, Qian W J, Gritsenko M A, Camp D G, 2nd, Monroe M E, Moore R J, Smith R D. Human plasma N-glycoproteome analysis by immunoaffinity subtraction, hydrazide chemistry, and mass spectrometry. J Proteome Res. 2005;4:2070-80.

24. Tang W H, Francis G S, Morrow D A, Newby L K, Cannon C P, Jesse R L, Storrow A B, Christenson R H, Apple F S, Ravkilde J, Wu A H. National Academy of Clinical Biochemistry Laboratory Medicine practice guidelines: Clinical utilization of cardiac biomarker testing in heart failure. Circulation. 2007;116:e99-e109.

25. Colucci W S, Elkayam U, Horton D P, Abraham W T, Bourge R C, Johnson A D, Wagoner L E, Givertz M M, Liang C S, Neibaur M, Haught W H, LeJemtel T H. Intravenous nesiritide, a natriuretic peptide, in the treatment of decompensated congestive heart failure. Nesiritide Study Group. N. Engl. J. Med. 2000;343:246-253.

26. Izumiya Y, Shiojima, I., Sato, K., Sawyer, D. B., Colucci, W. S. and Walsh, K. Vascular endothelial growth factor blockade promotes the transition from compensatory cardiac hypertrophy to failure in response to pressure overload. Hypertension. 2006;47:887-893.

27. Balemans W, Van Hul W. Extracellular regulation of BMP signaling in vertebrates: a cocktail of modulators. Dev. Biol. 2002;250:231-250.

28. Morissette M R, Cook, S. A., Foo, S., Mckoy, G., Ashida, N., Novikov, M., Scherrer-Crosbie, M., Li, L., Matsui, T., Brooks, G. and Rosenzweig, A. Myostatin regulates cardiomyocyte growth through modulation of Akt signaling. Circ. Res. 2006;99:15-24.

29. McKoy G, Bicknell K A, Patel K, Brooks G. Developmental expression of myostatin in cardiomyocytes and its effect on foetal and neonatal rat cardiomyocyte proliferation. Cardiovasc Res. 2007;74:304-12.

30. Cohn R D, Liang H Y, Shetty R, Abraham T, Wagner K R. Myostatin does not regulate cardiac hypertrophy or fibrosis. Neuromuscul Disord. 2007;17:290-6.

31. Artaza J N, Reisz-Porszasz S, Dow J S, Kloner R A, Tsao J, Bhasin S, Gonzalez-Cadavid N F. Alterations in myostatin expression are associated with changes in cardiac left ventricular mass but not ejection fraction in the mouse. J. Endocrinol. 2007;194:63-76.

32. Xu J, Kimball T R, Lorenz J N, Brown D A, Bauskin A R, Klevitsky R, Hewett T E, Breit S N, Molkentin J D. GDF15/MIC-1 functions as a protective and antihypertrophic factor released from the myocardium in association with SMAD protein activation. Circ Res. 2006;98:342-50.

33. Kempf T, Eden M, Strelau J, Naguib M, Willenbockel C, Tongers J, Heineke J, Kotlarz D, Xu J, Molkentin J D, Niessen H W, Drexler H, Wollert K C. The transforming growth factor-beta superfamily member growth-differentiation factor-15 protects the heart from ischemia/reperfusion injury. Circ Res. 2006;98:351-60.

34. Kempf T, von Haehling S, Peter T, Allhoff T, Cicoira M, Doehner W, Ponikowski P, Filippatos G S, Rozentryt P, Drexler H, Anker S D, Wollert K C. Prognostic utility of growth differentiation factor-15 in patients with chronic heart failure. J Am Coll Cardiol. 2007;50:1054-60.

35. Wollert K C, Kempf T, Peter T, Olofsson S, James S, Johnston N, Lindahl B, Horn-Wichmann R, Brabant G, Simoons M L, Armstrong P W, Califf R M, Drexler H, Wallentin L. Prognostic value of growth-differentiation factor-15 in patients with non-ST-elevation acute coronary syndrome. Circulation. 2007; 115:962-71.

36. Izumi M, Fujio Y, Kunisada K, Negoro S, Tone E, Funamoto M, Osugi T, Oshima Y, Nakaoka Y, Kishimoto T, Yamauchi-Takihara K, Hirota H. Bone morphogenetic protein-2 inhibits serum deprivation-induced apoptosis of neonatal cardiac myocytes through activation of the Smad1 pathway. J Biol Chem. 2001;276:31133-41.

37. Mashimo J, Maniwa, R., Sugino, H., and Nose, K. Decrease in the expression of a novel TGF beta1-inducible and ras-recision gene, TSC-36, in human cancer cells. Cancer Lett. 1997;113:213-219.

38. Bueno O F, De Windt, L. J., Tymits, K. M., Witt, S. A., Kimball, T. R., Klevitsky, R., Hewett, T. E., Jones, S. P., Lefer, D. J., Peng, C. F., Kitsis, R. N. and Molkentin, J. D. The MEK1-ERK1/2 signaling pathway promotes compensated cardiac hypertrophy in transgenic mice. EMBO J. 2000;19:6341-6350.

39. Cai Z, Zhong H, Bosch-Marce M, Fox-Talbot K, Wang L, Wei C, Trush M A, Semenza G L. Complete loss of ischaemic preconditioning-induced cardioprotection in mice with partial deficiency of HIF-1 {alpha}. Cardiovasc. Res. 2008;77:463-470.

40. Muraski J A, Rota M, Misao Y, Fransioli J, Cottage C, Gude N, Esposito G, Delucchi F, Arcarese M, Alvarez R, Siddiqi S, Emmanuel G N, Wu W, Fischer K, Martindale J J, Glembotski C C, Leri A, Kajstura J, Magnuson N, Berns A, Beretta R M, Houser S R, Schaefer E M, Anversa P, Sussman M A. Pim-1 regulates cardiomyocyte survival downstream of Akt. Nat. Med. 2007;13:1467-1475.

41. Das S, Fraga C G, Das D K. Cardioprotective effect of resveratrol via HO-1 expression involves p38 map kinase and PI-3-kinase signaling, but does not involve NFkappaB. Free Radic. Res. 2006;40:1066-1075.

42. Pimentel D R, Amin, J. K., Xiao, L., Miller, T., Viereck, J., Oliver-Krasinski, J., Baliga, R., Wang, J., Siwik, D. A., Singh, K., Pagano, P., Colucci, W. S., and Sawyer, D. B. Reactive oxygen species mediate amplitude-dependent hypertrophic and apoptotic responses to mechanical stretch in cardiac myocytes. Circ. Res. 2001;89:453-460.

43. Shibata R, Sato, K., Pimentel, D. R., Takemura, Y., Kihara, S., Ohashi, K., Funahashi, T., Ouchi, N., and Walsh, K. Adiponectin protects against myocardial ischemia-reperfusion injury through AMPK- and COX-2-dependent mechanisms. Nat. Med. 2005; 11:1096-1103.

44. Shibata R, Ouchi, N., Ito, M., Kihara, S., Shiojima, I., Pimentel, D. R., Kumada, M., Sato, K., Sciekofer, S., Ohashi, K., Funahashi, T., Colucci, W. S., and Walsh, K. Adiponectin-mediated modulation of hypertrophic signals in the heart. Nat. Med. 2004;10:1384-1389.

### Example 2

Follistatin-Like 1, a Secreted Muscle Protein, Promotes Angiogenesis through an Endothelial Nitric Oxide Synthesis-Dependent Mechanism in Ischemic Muscle

Akt1 Overexpression in Skeletal Muscle Increases Capillary Vessel Formation and Upregulates Fstl1 Expression.

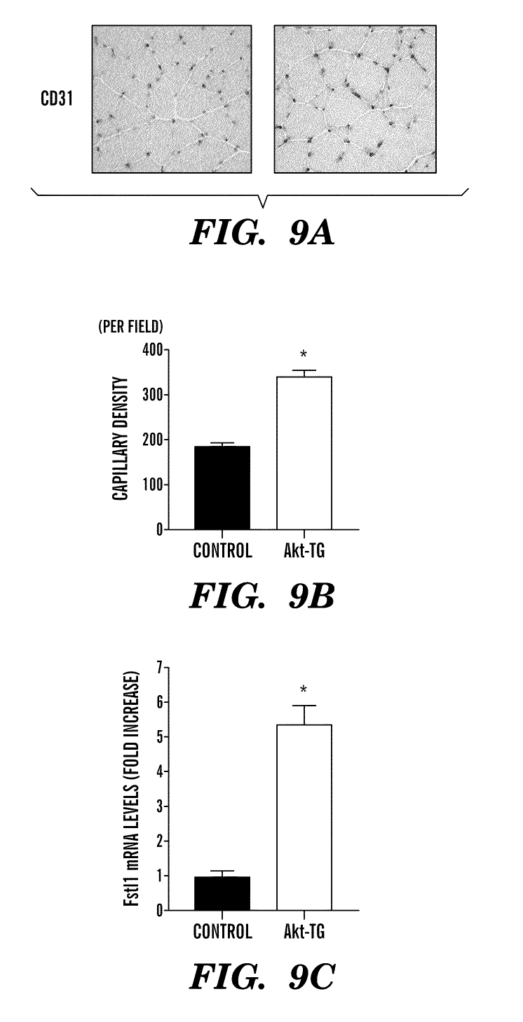

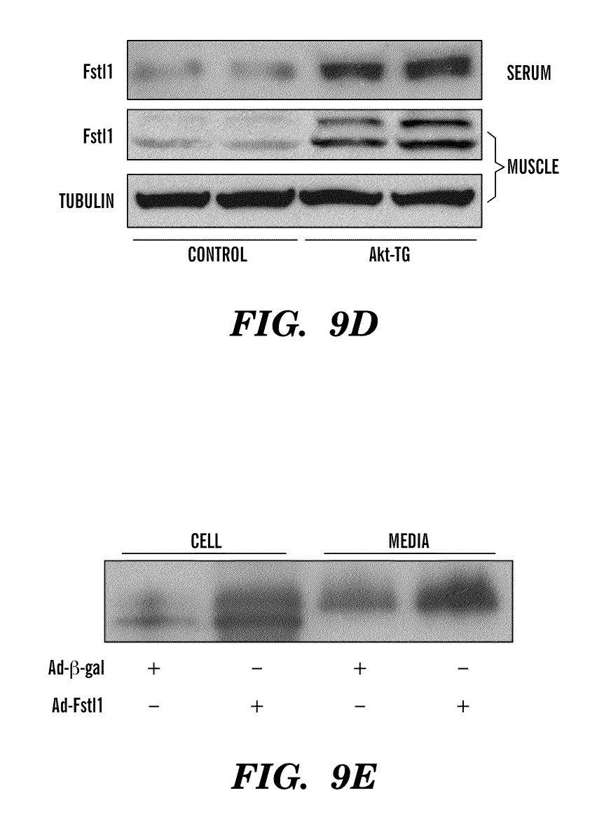

Initially the inventors examined the consequences of Akt1 transgene expression on blood vessel formation in skeletal muscle employing skeletal muscle-specific Akt1 TG mice (9). Mice were treated with DOX for 2 weeks to activate Akt1 transgene, resulting in an approximately 40% increase in gastrocnemius muscle mass weight (9). At this time point, capillary density in gastrocnemius muscle was assessed by staining with an endothelial marker CD31. FIG. 9A shows representative photographs of tissues stained with CD31. Quantitative analysis revealed that the capillary density was significantly increased in mice following 2 weeks of Akt1 activation compared with control mice (FIG. 9A).

Microarray analysis of expressed transcripts was performed, and transcripts upregulated by myogenic Akt signaling were evaluated for the presence of a signal peptide and the absence of a transmembrane domain in their open reading frames using Signal IP and SOUSI IP software, respectively. The Fstl1 transcript was of interest because it was upregulated by myogenic Akt signaling and it is predicted to encode a secreted protein. We next confirmed the effect of transgenic Akt1 activation on Fstl1 expression in skeletal muscle. Fstl1 mRNA levels were upregulated in gastrocnemius muscle by a factor of 5.3 following 2 weeks of Akt1 activation, as determined by QRT-PCR analysis (FIG. 9B). Fstl1 protein levels were also increased in gastrocnemius muscle by Akt1 transgene induction as assessed by Western blot analysis (FIG. 9B). Two immunoreactive bands of Fstl1 (37 kDa and 46 kDa proteins) were detected in mouse skeletal muscle. Fstl1 protein was also detected in mouse serum, and serum Fstl1 levels were markedly increased at 2 weeks after Akt transgene activation in skeletal muscle (FIG. 9B).

To examine whether Fstl1 is secreted from cultured muscle cells, differentiated C2C12 cells were treated with adenoviral vectors expressing Fstl1 (Ad-Fstl1) or β-galactosidase (Ad-β-gal). Fstl1 protein was detected in both the cell pellet lysate and media of control cells treated with Ad-β-gal (FIG. 9C). Ad-Fstl1 treatment increased Fstl1 protein levels in both the cell lysate and media (FIG. 9C). Collectively, these data demonstrate that that Fstl1 polypeptide is secreted from skeletal muscle.

Muscle Ischemia Upregulates Fstl1 Expression.

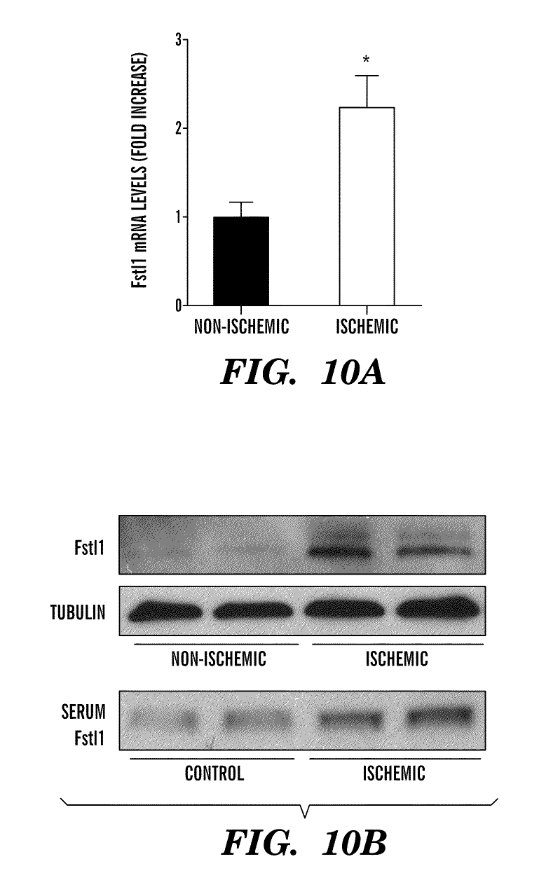

To further characterize the regulation of Fstl1, expression was determined in ischemic adductor muscle following femoral artery excision. Fstl1 mRNA levels were 2.3-fold higher in ischemic muscles than in non-ischemic muscles at 7 days after ischemic surgery as measured by QRT-PCR analysis (FIG. 10A). Fstl1 protein levels in ischemic muscles were also increased as assessed by Western blot analysis (FIG. 10). Furthermore, hindlimb ischemia increased serum Fstl1 levels at 2 weeks following ischemic surgery (FIG. 10B).

Fstl1 Promotes Angiogenesis in Response to Ischemia in Vivo.

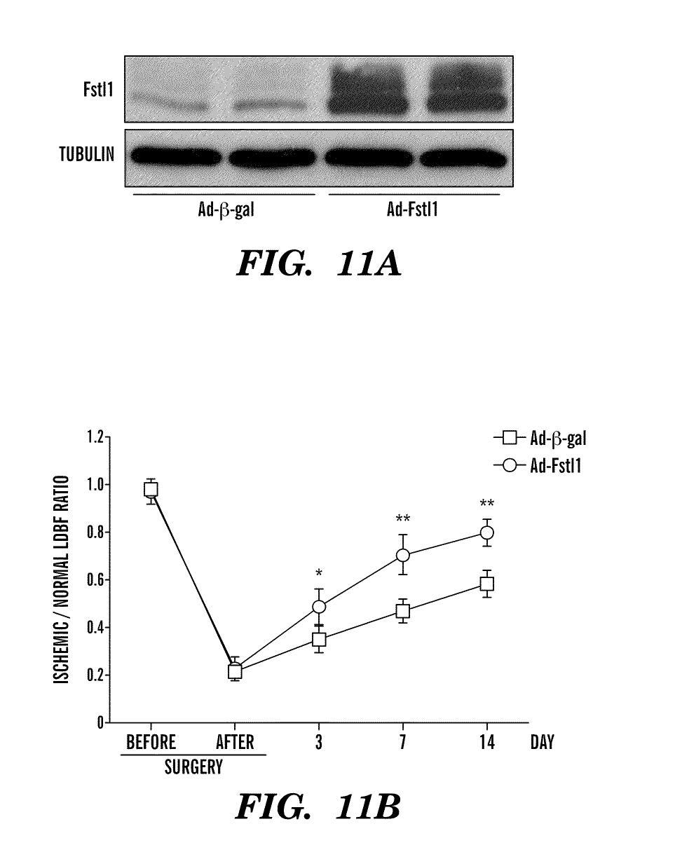

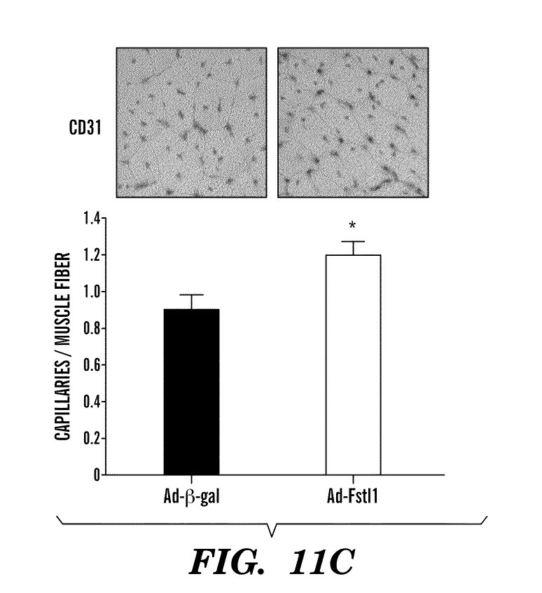

To test whether Fstl1 can modulate angiogenesis in vivo, the inventors employed C57BL/6 wild-type mice that underwent unilateral femoral artery resection. This model of vascular insufficiency has been used to evaluate the in vivo angiogenic actions of growth factors including VEGF (22,23). Adenoviral vectors expressing Fstl1 (Ad-Fstl1) and Ad-β-gal (control) were injected intramuscularly into the adductor muscle 3 days before surgery. Fstl1 protein levels in ischemic muscle were significantly elevated at 6 days after injection of Ad-Fstl1 (FIG. 11A). Ad-Fstl1-treated mice showed a significant increase in flow recovery at 3, 7 and 14 days after ischemic surgery as determined by laser Doppler blood flow analysis (FIG. 11B). To investigate the extent of revascularization at the microcirculatory level, capillary density was measured in histological sections harvested from the ischemic muscles. Quantitative analysis revealed that the capillary density was significantly increased in Ad-Fstl1-treated mice compared with control mice on postoperative day 14 (FIG. 11C).

Fstl1 Promotes Angiogenic Cellular Responses in Vitro.

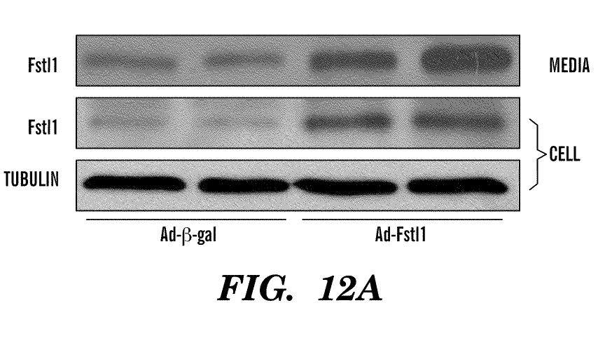

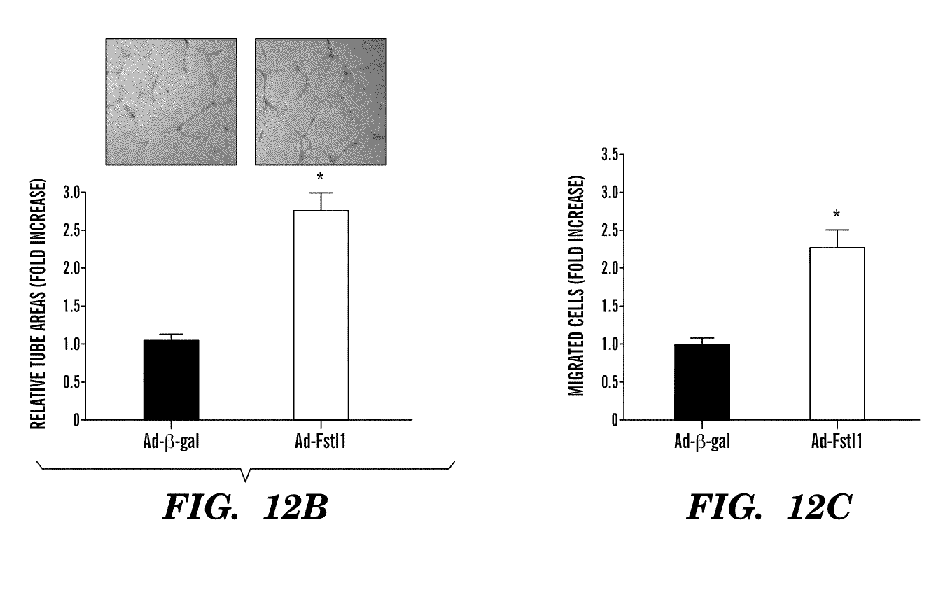

To examine whether Fstl1 can directly act on endothelial cells, HUVECs were transduced with Ad-Fstl1 or Ad-β-gal and plated on a Matrigel matrix. Fstl1 protein expression was readily detected in both cell lysate and media from HUVECs treated with Ad-Fstl1, whereas endogenous levels of Fstl1 expression were low (FIG. 12A). Quantitative analyses of endothelial cell network area revealed that treatment with Ad-Fstl1 significantly promoted the formation of network structures relative to control cultures treated with Ad-β-gal (FIG. 12A). To test whether Ad-Fstl1 influences endothelial cell migration, a modified Boyden chamber assay was performed. Ad-Fstl1 treatment significantly stimulated HUVEC migration in this assay (FIG. 12B).

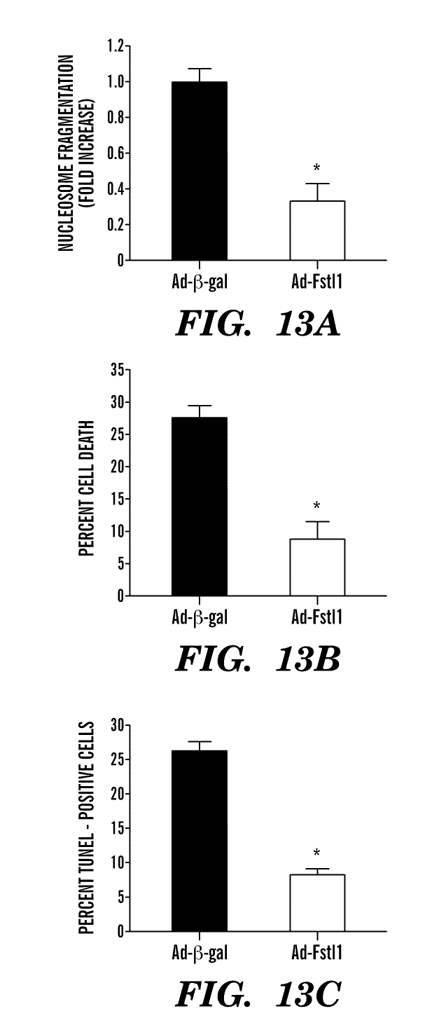

To evaluate the role of Fstl1 in endothelial apoptosis, HUVECs were treated with Ad-Fstl1 or Ad-β-gal followed by 48 hours of incubation in serum-free media. Fstl1 markedly suppressed the extent of nucleosome fragmentation as determined by ELISA (FIG. 13A). Ad-Fstl1 also reduced HUVEC death caused by serum-deprivation as assessed by an MTS-based assay (FIG. 13B). To corroborate these findings, TUNEL-positive cells were analyzed in the HUVEC cultures. As shown in the FIG. 13C, treatment with Ad-Fstl1 diminished the frequency of TUNEL-positive cells under serum-deprived conditions.

Fstl1 Stimulates the Phosphorylation of Akt and eNOS.

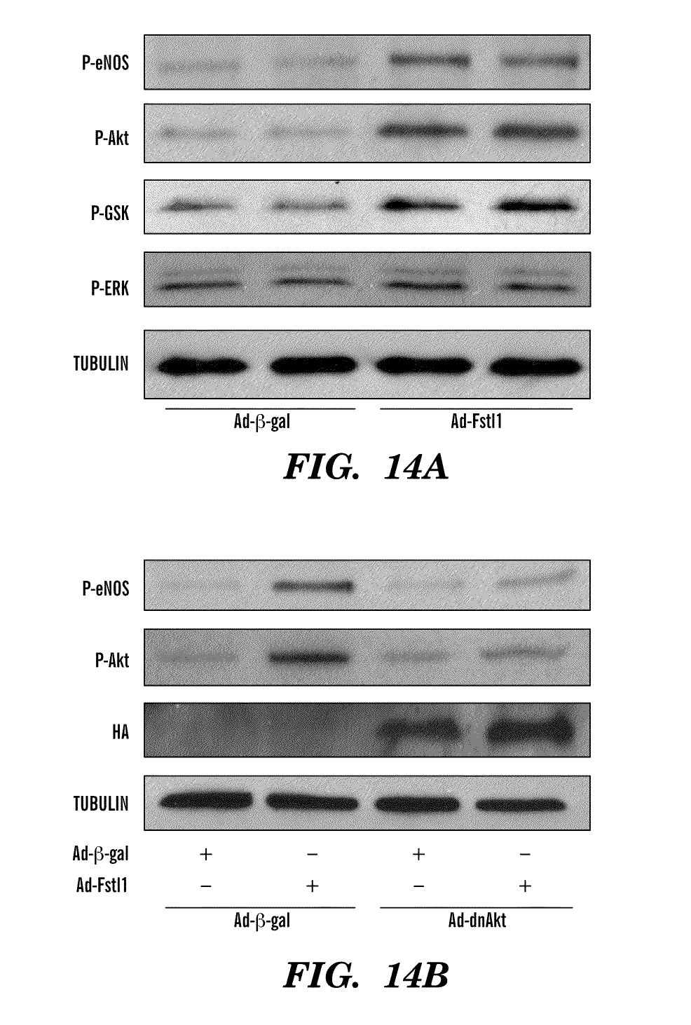

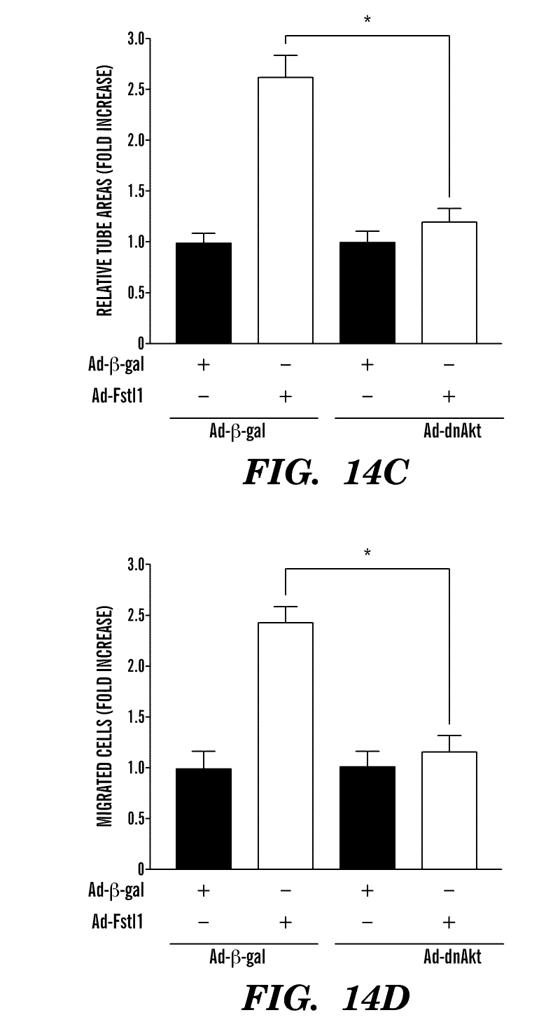

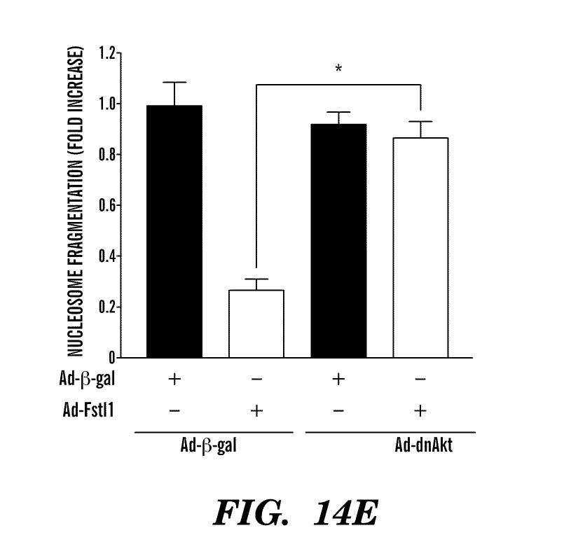

Akt has been shown to be a key mediator of growth factor-dependent angiogenic and survival signals in endothelial cells (19,24). Therefore, to test whether Fstl1 influences Akt signaling in endothelial cells, the activating phosphorylation of Akt at Ser473 was assessed by Western blot analysis. Treatment of HUVECs with Ad-Fstl1 enhanced the phosphorylation of Akt (FIG. 14A). Since Akt can phosphorylate eNOS at Ser1179 (25,26), eNOS phosphorylation was also examined in these cultures. Ad-Fstl1 stimulation resulted in an increase in eNOS phosphorylation at Ser1179 (FIG. 14A). Consistent with an increase in Akt signaling, and increase in GSK-3β phosphorylation at Ser9, a downstream target of Akt signaling in endothelial cells (27), was seen under these conditions (FIG. 6A). In contrast, Ad-Fstl1 had no effect on the phosphorylation of ERK at Thr202/Tyr204 (FIG. 6A). To examine the role of Akt in the regulation of eNOS phosphorylation by Fstl1, HUVECs were infected with a HA-tagged dominant-negative Akt (Ad-dnAkt) or Ad-β-gal. Transduction with Ad-dnAkt reduced Fstl1-induced Akt and eNOS phosphorylation (FIG. 14B). These data indicate that Akt mediates eNOS phosphorylation downstream from Fstl1.

Akt-eNOS Signaling is Required for the Pro-Angiogenic and Anti-Apoptotic Actions of Fstl1.

To test whether the activation of Akt signaling is required for Fstl1-stimulated endothelial differentiation, migration and survival, HUVECs were infected with Ad-dnAkt or Ad-β-gal, and angiogenic and survival activities were assessed by differentiation, migration and nucleosome fragmentation assays. Transduction with Ad-dnAkt blocked Ad-Fstl1-induced network formation by HUVECs plated on Matrigel (FIG. 14C). Ad-Fstl1-stimulated endothelial cell migration was also diminished by transduction with Ad-dnAkt, whereas Ad-dnAkt had no effect on basal migration (FIG. 14D). Furthermore, transduction with Ad-dnAkt reversed the inhibitory effects of Ad-Fstl1 on the degree of nucleosome fragmentation (FIG. 14E). These results indicate that Akt signaling is required for Fstl1-induced endothelial cell differentiation, migration and survival.

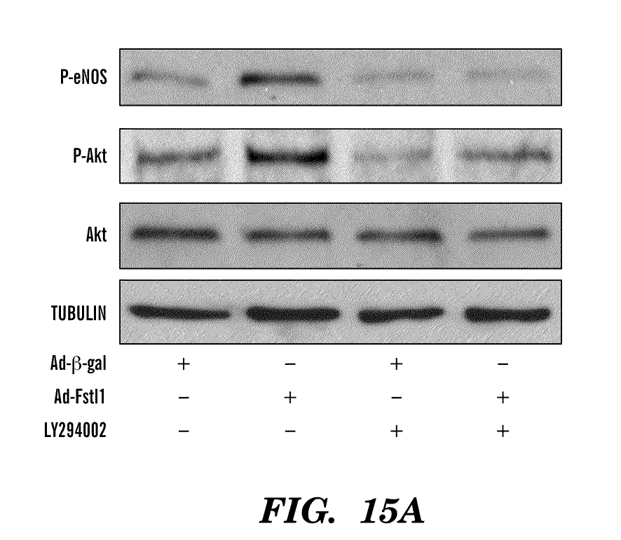

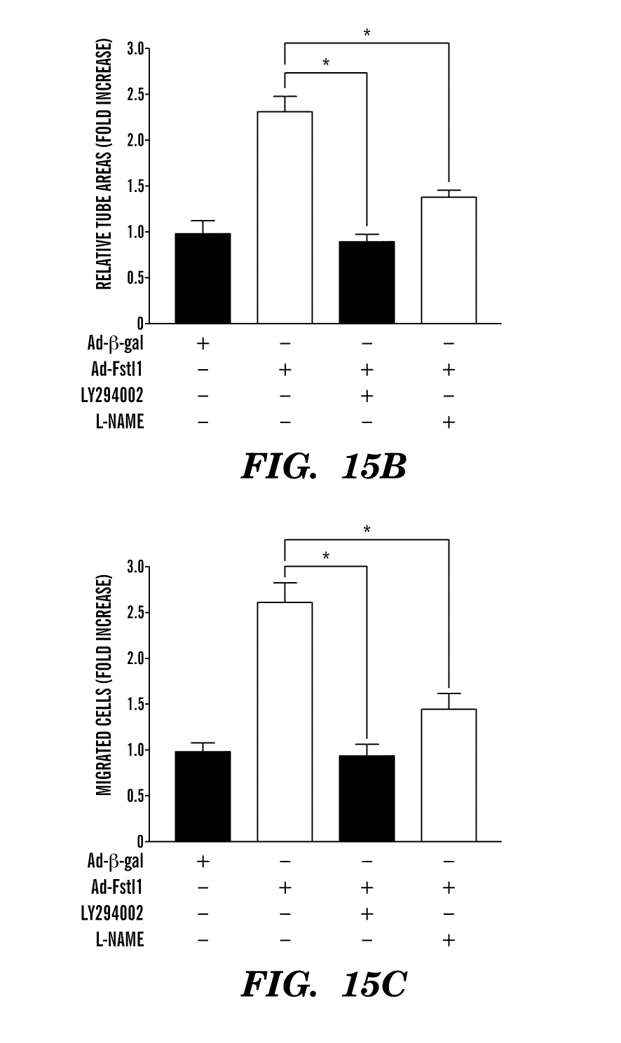

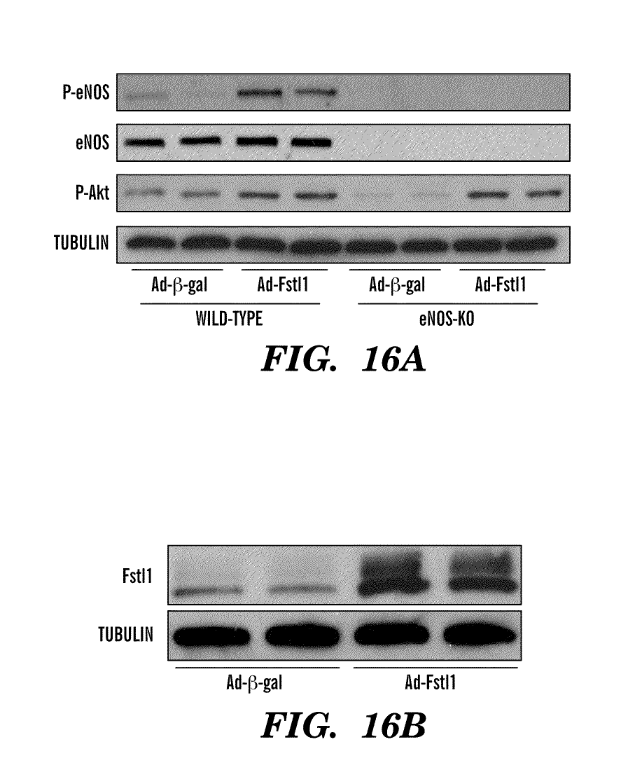

Akt is activated by many growth factors through the phosphatidylinositol-3 kinase (PI3-kinase)-dependent pathway (24). To investigate whether PI3-kinase participates in Fstl1-induced angiogenic signaling, HUVECs were treated with PI3-kinase inhibitor LY294002. Treatment with LY294002 abolished Ad-Fstl1-stimulated phosphorylation of Akt and eNOS in HUVECs (FIG. 15A). Ad-Fstl1-stimulated network formation and migration of HUVECs were also blocked by treatment with LY294002 (FIG. 15B and C). These data indicate that PI3-kinase is essential for the angiogenic cellular responses to Fstl1 and that PI3-kinase functions upstream from the Akt-eNOS regulatory axis in Fstl1-stimulated endothelial cells.

To test the contribution of eNOS to the angiogenic responses of Fstl1, HUVECs were treated with the NOS inhibitor L-NAME. L-NAME treatment significantly reduced Ad-Fstl1-induced endothelial cell differentiation into network structures and migration (FIG. 15B and C), indicating that Fstl1 promotes pro-angiogenic properties in an eNOS-dependent manner.

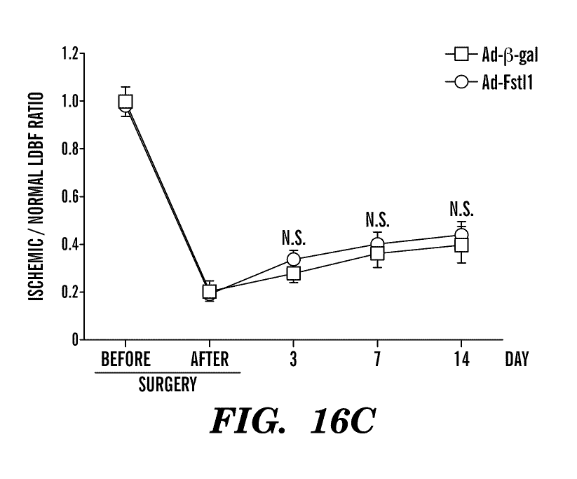

Role of eNOS signaling in Fstl1-stimulated angiogenesis in vivo. To analyze the potential role of eNOS activation in Fstl1-mediated regulation of angiogenesis in vivo, the phosphorylation status of eNOS and Akt in ischemic muscles of C57/BL6 mice was assessed by Western blot analysis at day 6 after intramuscular injection of Ad-Fstl1 and Ad-β-gal. Ad-Fstl1 treatment stimulated eNOS phosphorylation at serine residue 1177 in ischemic adductor muscle without affecting total eNOS protein levels (FIG. 16A). Akt phosphorylation at Ser473 in ischemic muscles was also stimulated by Ad-Fstl1 treatment (FIG. 16A).

To assess the contribution of eNOS signaling to the stimulatory actions of Fstl1 on ischemia-driven angiogenesis in vivo, the inventors intramuscularly injected Ad-Fstl1 or Ad-β-gal into eNOS-knockout (eNOS-KO) mice 3 days before induction of ischemia. At 6 days after Ad-Fstl1 infection, Fstl1 protein levels in ischemic muscles of eNOS-KO mice increased to the similar levels that were observed previously in wild-type C57/BL6 mice (FIG. 16B). Akt phosphorylation in ischemic muscles of eNOS-KO mice was stimulated by Ad-Fstl1 treatment to a similar extent compared to that of wild-type mice (FIG. 16A). However, in contrast to wild-type mice (FIG. 11B), treatment with Ad-Fstl1 did not promote flow recovery in ischemic hindlimbs in eNOS-KO mice (FIG. 16C). Thus the angiogenic-stimulatory activity of Fstl1 in vivo is dependent on eNOS.

The inventors herein discovered that Fstl1 plays a role in the promotion of new blood vessel growth. Fstl1 expression in muscle was upregulated by Akt1 transgene activation during muscle hypertrophy and by ischemic injury. Fstl1 overexpression was shown to enhance endothelial cell differentiation and migration, and diminish endothelial cell apoptosis, through its ability to activate Akt-eNOS signaling pathways within these cells. Administration of Fstl1 improved revascularization in ischemic limbs of mice of wild-type but not eNOS-deficient mice. The ability of Fstl1 to stimulate Akt-eNOS signaling within endothelial cells contributes to the stimulation of angiogenesis in this model.

It is well documented that Akt-eNOS signaling participates in regulation of blood vessel growth and vascular cell survival (19,24,28). Herein, the inventors demonstrate that the stimulation of angiogenesis by Fstl1 is dependent on its ability to activate Akt-eNOS signaling in endothelial cells. Fstl1 stimulated the activating phosphorylation of Akt and eNOS in endothelial cells, whereas transduction with dominant-negative Akt reduced Fstl1-stimulated endothelial cell differentiation, migration, survival and eNOS phosphorylation. Inhibitors of PI3-kinase or eNOS blocked the increase in endothelial differentiation and migration caused by Fstl1. The pro-angiogenic consequences of Fstl1 overexpression in ischemic muscle was associated with increased eNOS phosphorylation at Ser1177, and the angiogenic actions of Fstl1 were abolished in eNOS-KO mice. Collectively, these observations suggest that the Fstl1-Akt-eNOS regulatory signaling axis functions to stimulate vascular cell function under ischemic conditions, thereby promoting vascular growth.

It has been proposed that skeletal muscle secretes factors, commonly referred to as “myokines” in the art, that influence the behavior of neighboring or remote cells (29). Several lines of evidence indicate that Fstl1 can be designated a pro-angiogenic. Fstl1 is secreted into the media by cultured skeletal muscle cells, and it can directly act on endothelial cells leading to the induction of an angiogenic phenotype. Furthermore, both Akt transgene-induced myofiber hypertrophy and ischemic hindlimb surgery also led to an increase in both tissue-resident and serum levels of Fstl1. It is widely recognized that tissue ischemia will lead to the upregulation of multiple angiogenic growth factors that function to coordinate the repair of the vascular network (11). It is also recognized that skeletal muscle hypertrophy is coupled to angiogenesis, through molecular mechanisms that are independent tissue hypoxia (6). As such that the upregulation and secretion of Fstl1 by skeletal muscle, under conditions of hypertrophic growth or ischemic stress, contributes to blood vessel growth through its ability to modulate the behavior of endothelial cells.

Herein, the inventors have demonstrated that Fstl1 is upregulated by Akt activation during cardiac hypertrophy (See Example 1 above). In this study, Fstl1 was demonstrated to activate Akt signaling in cardiac myocytes and inhibit apoptosis. Thus, Fstl1 can function as a survival factor for both cardiac myocytes and endothelial cells via an activation of Akt signaling. Fstl1 overexpression has also been shown to protect the heart from ischemia-reperfusion injury in mice. Treatments aimed at increasing angiogenesis represent a promising strategy for treatment of ischemic limb and heart diseases (30). Thus, Fstl1 can serve as a therapeutic agent for ischemic diseases based upon its ability to directly stimulate blood vessel formation and inhibit the death of cardiovascular cells.

Other members of the follistatin family function to regulate TGF-β superfamily proteins through their ability to function as binding partners (31). Activin A has been shown to suppress endothelial cell growth and attenuate angiogenesis by a chorioallantoic membrane assay (32). In this setting, follistatin appears to promote angiogenesis through its ability to bind to Activin (33). However, it remains to be determined whether Fstl1 binds to members of TGF-β superfamily in a manner that is similar to follistatin (34). In this regard Fstl1 exhibits little amino acid sequence homology with follistatin (7%), and our in vitro data show that overexpression of Fstl1 results in enhanced Akt signaling and increased angiogenic responses in cultured endothelial cells under conditions of serum deprivation. It is unlikely that the actions of Fstl1 on endothelial cell signaling and phenotype are mediated by its ability to modulate the function of a second secreted protein. Thus, attempts are currently in progress to identify the Fstl1 receptor in endothelial cells.

In conclusion, the inventors have demonstrated that Fstl1 is a myokine that functions to promote angiogenesis through its ability to activate Akt-eNOS signaling in endothelial cells. Overexpression of Fstl1 stimulates ischemia-induced angiogenesis in mice through activation of eNOS. Because the dysregulated eNOS signaling is linked to endothelial dysfunction, impaired neovascularization and atherogenesis (35-37), strategies to increase Fstl1-eNOS signaling will be a useful treatment for vascular complications.

(The abbreviations used are: VEGF, vascular endothelial growth factor; HUVECs, Human umbilical vein endothelium cells; Fstl1, Follistatin-like 1; eNOS, endothelial nitric oxide synthase; PI3-kinase, phosphatidylinositol-3 kinase; eNOS-KO, eNOS-knockout.)

### References for Example 2

The references below refer to references cited in Example 2 and all reference numbers cited in brackets, i.e. “(1)” in Example 2 and the “material and methods” section of the Examples section. All references described herein are incorporated herein by reference in their entirety.

Degens, H., Veerkamp, J. H., van Moerkerk, H. T. B., Turek, Z., Hoofd, L. J. C., and Binkhorst, R. A. (1993) Int. J. Biochem. 25, 1141-1148

Degens, H., Turek, Z., Hoofd, L. J. C., Van't Hof, M. A., and Binkhorst, R. A. (1992) J. Anat. 180, 455-463

Ingjer, F. (1979) Eur. J. Appl. Physiol. Occup. Physiol. 40, 197-209

Kano, Y., Shimegi, S., Masuda, K., Ohmori, H., and Katsuta, S. (1997) Eur. J. Appl. Physiol. Occup. Physiol. 75, 97-101

Shiojima, I., and Walsh, K. (2006) Genes Dev 20, 3347-3365

Takahashi, A., Kureishi, Y., Yang, J., Luo, Z., Guo, K., Mukhopadhyay, D., Ivashchenko, Y., Branellec, D., and Walsh, K. (2002) Mol. Cell. Biol. 22, 4803-4814

Lai, K. M., Gonzalez, M., Poueymirou, W. T., Kline, W. O., Na, E., Zlotchenko, E., Stitt, T. N., Economides, A. N., Yancopoulos, G. D., and Glass, D. J. (2004) Mol. Cell. Biol. 24, 9295-9304

Bodine, S. C., Stitt, T. N., Gonzalez, M., Kline, W. O., Stover, G. L., Bauerlein, R., Zlotchenko, E., Scrimgeour, A., Lawrence, J. C., Glass, D. J., and Yancopoulos, G. D. (2001) Nat. Cell Biol. 3, 1014-1019

Izumiya, Y., Hopkins, T., Morris, C., Sato, K., Zeng, L., Viereck, J., Hamilton, J. A., Ouchi, N., LeBrasseur, N. K., and Walsh, K. (2008) Cell Metab 7, 159-172

Shiojima, I., Sato, K., Izumiya, Y., Schiekofer, S., Ito, M., Liao, R., Colucci, W., and Walsh, K. (2005) J. Clin. Invest. 115, 2108-2118

Carmeliet, P. (2005) Nature 438, 932-936

Shibanuma, M., Mashimo, J., Mita, A., Kuroki, T., and Nose, K. (1993) Eur J Biochem 217, 13-19

Sumitomo, K., Kurisaki, A., Yamakawa, N., Tsuchida, K., Shimizu, E., Sone, S., and Sugino, H. (2000) Cancer Lett 155, 37-46

Johnston, I. M., Spence, H. J., Winnie, J. N., McGarry, L., Vass, J. K., Meagher, L., Stapleton, G., and Ozanne, B. W. (2000) Oncogene 19, 5348-5358

Oshima, Y., Ouchi, N., Sato, K., Izumiya, Y., Pimentel, D., and Walsh, K. (2008) Circulation, In press.

Ouchi, N., Shibata, R., and Walsh, K. (2005) Circ. Res. 96, 838-846

Shibata, R., Ouchi, N., Kihara, S., Sato, K., Funahashi, T., and Walsh, K. (2004) J. Biol. Chem. 279, 28670-28674

Ouchi, N., Kobayashi, H., Kihara, S., Kumada, M., Sato, K., Inoue, T., Funahashi, T., and Walsh, K. (2004) J. Biol. Chem. 279, 1304-1309

Fujio, Y., and Walsh, K. (1999) J. Biol. Chem. 274, 16349-16354

Kobayashi, H., Ouchi, N., Kihara, S., Walsh, K., Kumada, M., Abe, Y., Funahashi, T., and Matsuzawa, Y. (2004) Circ. Res. 94, e27-e31

Shibata, R., Sato, K., Pimentel, D. R., Takemura, Y., Kihara, S., Ohashi, K., Funahashi, T., Ouchi, N., and Walsh, K. (2005) Nat. Med. 11, 1096-1103

Couffinhal, T., Silver, M., Kearney, M., Sullivan, A., Witzenbichler, B., Magner, M., Annex, B., Peters, K., and Isner, J. M. (1999) Circulation 99, 3188-3198

Rivard, A., Silver, M., Chen, D., Kearney, M., Magner, M., Annex, B., Peters, K., and Isner, J. M. (1999) Am. J. Pathol. 154, 355-363

Shiojima, I., and Walsh, K. (2002) Circ. Res. 90, 1243-1250

Fulton, D., Gratton, J. P., McCabe, T. J., Fontana, J., Fujio, Y., Walsh, K., Franke, T. F., Papapetropoulos, A., and Sessa, W. C. (1999) Nature 399, 597-601

Dimmeler, S., Fleming, I., Fisslthaler, B., Hermann, C., Busse, R., and Zeiher, A. Z. (1999) Nature 399, 601-605

Kim, H.-S., Skurk, C., Thomas, S. R., Bialik, A., Suhara, T., Kureishi, Y., Birnbaum, M., Keaney Jr., J. F., and Walsh, K. (2002) J. Biol. Chem. 277, 41888-41896

Kureishi, Y., Luo, Z., Shiojima, I., Bialik, A., Fulton, D., Lefer, D. J., Sessa, W. C., and Walsh, K. (2000) Nat. Med. 6, 1004-1010

Pedersen, B. K., Akerstrom, T. C., Nielsen, A. R., and Fischer, C. P. (2007) J. Appl. Physiol. 103, 1093-1098

Vale, P. R., Isner, J. M., and Rosenfield, K. (2001) J. Interv. Cardiol. 14, 511-528

Balemans, W., and Van Hul, W. (2002) Dev Biol 250, 231-250

Breit, S., Ashman, K., Wilting, J., Rossler, J., Hatzi, E., Fotsis, T., and Schweigerer, L. (2000) Cancer Res 60, 4596-4601

Kozian, D. H., Ziche, M., and Augustin, H. G. (1997) Lab Invest 76, 267-276

Mashimo, J., Maniwa, R., Sugino, H., and Nose, K. (1997) Cancer Lett 113, 213-219

Kawashima, S., and Yokoyama, M. (2004) Arterioscler Thromb Vasc Biol 24, 998-1005

Forstermann, U., and Munzel, T. (2006) Circulation 113, 1708-1714

Murohara, T., Asahara, T., Silver, M., Bauters, C., Masuda, H., Kalka, C., Kearney, M., Chen, D., Symes, J. F., Fishman, M. C., Huang, P. L., and Isner, J. M. (1998) J. Clin. Invest. 101, 2567-2578

### Example 3

Follistatin Like 1 Levels as a Metabolic Diagnostic or Marker of Metabolic Dysfunction

Fstl1 Expression in Muscle is Downregulated upon Aging, Starvation and Metabolic Dysfunction

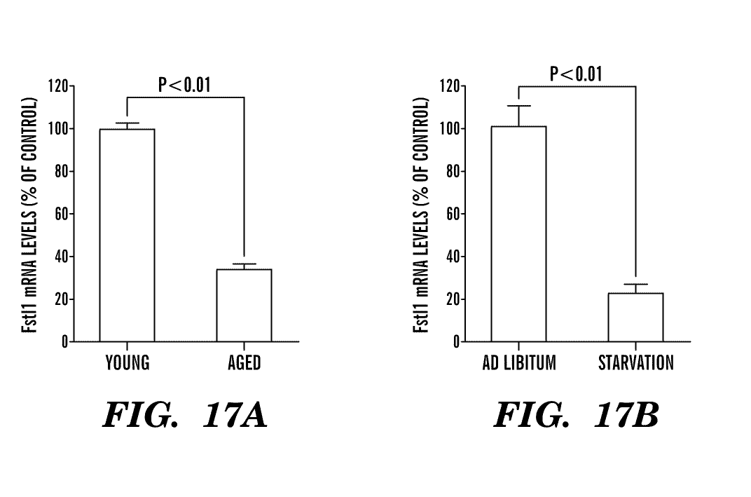

Fstl1 transcript levels were examined in young (2 mo.) and aged (2 yr) mice. Old mice expressed less Fstl1 (FIG. 17A). Fstl1 transcript levels were also downregulated in muscle in response to food deprivation for 48 hrs (FIG. 17B).

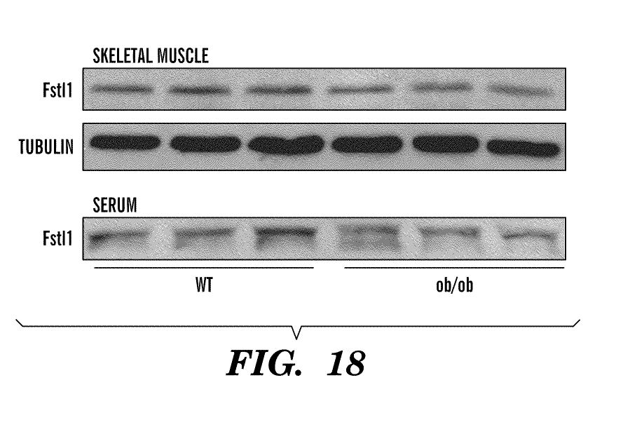

The ob/ob homozygous mouse is leptin deficient, hyper-insulinemic and becomes extremely obese. The ob/ob mice exhibit hyperphagia, a diabetes-like syndrome of hyperglycemia, glucose intolerance, elevated plasma insulin. These mice serve as excellent animal models for human obesity and related insulin disorders. Fstl1 expression in these mice was investigated and compared to levels in wild-type mice. Fstl1 protein in muscle and serum was significantly downregulated in these obese, diabetic ob/ob mice compared to wild-type mice (FIG. 18).

Fstl1 Protein can be Detected in Human Serum

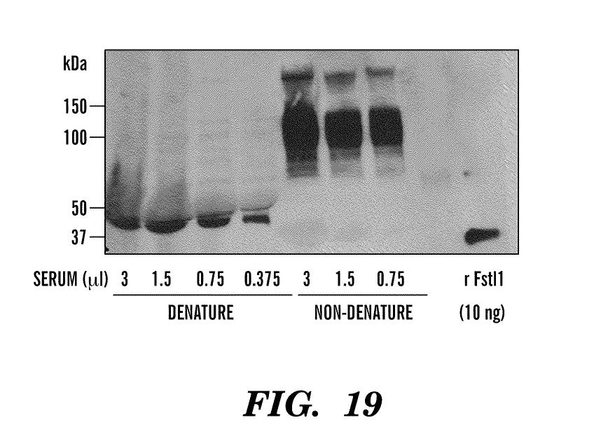

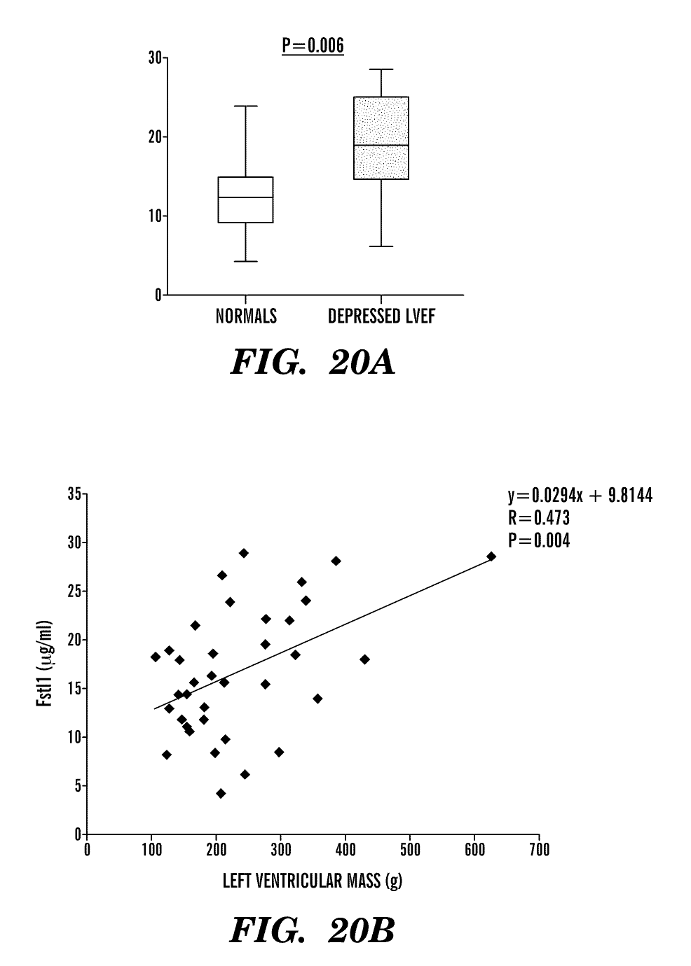

Fstl1 was readily detected by western blot analysis in denatured human serum obtained from a healthy individual (FIG. 19). Furthermore, the inventors have demonstrated in FIGS. 20A and 20B clinical data showing high levels of Fstl1 protein in the serum of patients with heart failure, demonstrating that unlike other markers for cardiac injury, Fstl1 polypeptide is secreted by heart cells (i.e. myocytes) under stress. Accordingly, monitoring the presence of Fstl1 polypeptide in a biological sample from a subject, such as serum sample is useful in the clinic as a prognostic and/or diagnostic marker for a subject at risk of a myocardial infarction and/or heart failure.

All references described herein are incorporated herein by reference in their entirety.

What is claimed is:

1. A system for analyzing a biological sample from a subject comprising:
a) a determination module configured to receive a biological sample and to determine Fstl1 expression level information, wherein the Fstl1 expression level information, comprises:
(i) the Fstl1 polypeptide expression level in the biological sample; and/or
(ii) the Fstl1 gene expression level in the biological sample;
b) a connection from the determination module to transmit the Fstl1 expression level information to an electronic computer, wherein the computer comprises a storage device, a comparison module and a display module;
c) the storage device configured to store Fstl1 expression level information from the determination module;
d) the comparison module adapted to compare the Fstl1 expression level information stored on the storage device with reference data, and to provide a comparison result, wherein the comparison result comprises;
(i) a comparison of the Fstl1 expression level in the biological sample with the reference Fstl1 expression level, and
(ii) a determination of the Fstl1 expression level in the biological sample above or below a threshold level relative to the reference Fstl1 expression level, wherein a Fstl1 expression level above the threshold level is indicative of cardiac stress and/or skeletal muscle stress; and wherein a Fstl1 expression level below the threshold level is indicative of diabetes; and/or metabolic disorder;
e) the display module for displaying a content based in part on the comparison result for the user, wherein the content is a signal indicative of the likelihood of at least one of: cardiac stress and/or skeletal muscle stress and/or diabetes; and/or a metabolic disorder in the subject.

2. The system of claim 1, wherein the threshold level that is indicative of cardiac stress or skeletal muscle stress is at least 30%.

3. The system of claim 1, wherein the Fstl1 polypeptide expression level is the expression level of the polypeptide of SEQ ID NO: 1.

4. The system of claim 1, wherein the Fstl1 gene expression level is the expression level of the nucleotide of SEQ ID NO: 2 or expression level of Fstl1 mRNA.

5. The system of claim 1, wherein the biological sample is selected from the group consisting of: a muscle sample, a tissue sample, a biopsy sample, an ex vivo cultivated sample, a ex vivo cultivated tissue sample, a surgically dissected tissue sample, a blood sample, a plasma sample, a lymph fluid sample, a primary ascite sample, a serum sample, a sputum sample, a saliva sample, a stool sample, a urine sample, a lymph fluid sample, a tear sample, a milk sample.

6. The system of claim 1, wherein the biological sample is obtained from a mammalian subject or a human subject.

7. The system of claim 1, wherein the cardiac injury is a myocardial infarction.

8. The system of claim 1, wherein the Fstl1 polypeptide expression is measured by immuno assay.

9. The system of claim 8, wherein the immuno assay is Western blot analysis or ELISA.

10. The system of claim 5, wherein the tissue sample is from an organ, skeletal muscle, neuronal tissue.

11. The system of claim 5, wherein the tissue sample is a cardiac tissue sample or a skeletal muscle tissue sample.

12. The system of claim 1, wherein the subject has experienced one or more symptoms or risk factors for one or more of pressure overload hypertrophy, myocardial infarction, angina pectoris (stable and unstable), cardiomyopathy, myocarditis, congestive heart failure, reinfarction, atherosclerosis, acute coronary syndrome, idiopathic cardiomyopathy, drug-induced cardiomyopathy, alcoholic cardiomyopathy, hypertensive cardiomyopathy, atheromatous disorders of the major blood vessels, metabolic cardiomyopathy, diabetic cardiomyopathy. Ischemic cardiomyopathy, hypertensive cardiomyopathy, dilated cardiomyopathy, hypertrophic cardiomyopathy, arrhythmogenic right ventricular cardiomyopathy, left ventricular hypertrophy, cardiac arrhythmias. restrictive cardiomyopathy, inflammatory cardiomyopathy.

13. The system of claim 1, wherein the biological sample is taken from the subject following administration of a cardioprotective agent or a cardioprotective agent which increases the subject's Fstl1.

14. The system of claim 1, wherein the a determination module is configured to further determining the level of an additional agent in the biological sample.

15. The system of claim 14, wherein the additional agent is a marker of cardiac stress, and/or injury or both.

16. The system of claim 1, wherein the skeletal muscle distress is ischemic muscle disease.

17. The system of claim 1, wherein the subject is at risk of developing, or has a skeletal muscle ischemia disease.

18. The system of claim 17, wherein the skeletal muscle ischemia disease is selected from the group consisting of: muscular dystrophy, muscle atrophy, sarcopenia, inclusion body myocytis.

19. A computer readable medium having computer readable instructions recorded thereon to define software modules including a comparison module and a display module for implementing a method on a computer, said method comprising:
a) comparing with the comparison module the data stored on a storage device with reference data to provide a comparison result, wherein the comparison result is the Fstl1 expression level in the biological above a threshold level relative to a reference Fstl1 expression level that is indicative of cardiac stress or skeletal muscle stress; and
b) displaying a content based in part on the comparison result for the user, wherein the content is a signal indicative of cardiac stress and/or skeletal muscle stress.

20. The computer readable medium of claim 19, wherein the threshold level that is indicative of cardiac stress or skeletal muscle stress is 30%.

21. The computer readable medium of claim 19, wherein the Fstl1 polypeptide expression level is the expression level of the polypeptide of SEQ ID NO: 1.

22. The computer readable medium of claim 19, wherein the Fstl1 gene expression level is the expression level of the nucleotide of SEQ ID NO: 2 or expression level of Fstl1 mRNA.

23. The computer readable medium of claim 19, wherein the biological sample is selected from the group consisting of: a muscle sample, a tissue sample, a biopsy sample, an ex vivo cultivated sample, a ex vivo cultivated tissue sample, a surgically dissected tissue.

24. A method of treating a subject at risk for cardiac stress and/or skeletal muscle stress and/or diabetes and/or metabolic dysfunction comprising;
a. determining if the subject is at risk for cardiac stress and/or skeletal muscle stress and/or diabetes and/or metabolic dysfunction by measuring Fstl1 expression level in a biological sample obtained from the subject,
wherein high levels of Fstl1 polypeptide expression or Fstl1 gene expression or Fstl1 mRNA expression in the biological sample indicates the subject is likely to be at risk for cardiac stress and/or skeletal muscle stress, and
wherein low levels of Fstl1 polypeptide expression or Fstl1 gene expression or Fstl1 mRNA expression in the biological sample indicates the subject is likely to be at risk for diabetes and/or metabolic dysfunction,
b. administering to a subject determined to be at risk for cardiac stress and/or skeletal muscle stress a cardioprotective drug or skeletal muscle stress drug, or administering to a subject determined to be at risk for diabetes and/or metabolic dysfunction an appropriate therapy.

25. The method of claim 24, wherein the skeletal muscle stress drug is a nutritional program.
